(19) United States
(12) Patent Application Publication
(10) Pub. No.: US 2016/0296507 A1
(43) Pub. Date: Oct. 13, 2016

(54) IMIDAZOPYRIDINE, IMIDAZOPYRIMIDINE AND IMIDAZOPYRAZINE DERIVATIVES AS MELANOCORTIN-4 RECEPTOR MODULATORS

(71) Applicant: UNIVERSITE DE MONTREAL, Montreal (CA)

(72) Inventors: Réjean RUEL, Saint-Lambert (CA); Yves CHANTIGNY, Pincourt (CA); Anne MARINIER, Kirkland (CA); Patricia RENE, Montreal (CA); Michel BOUVIER, Montreal (CA)

(73) Assignee: UNIVERSITE DE MONTREAL, Montreal (CA)

(21) Appl. No.: 15/188,090

(22) Filed: Jun. 21, 2016

Related U.S. Application Data
(63) Continuation of application No. 13/808,698, filed on Mar. 15, 2013, now abandoned.
(60) Provisional application No. 61/367,558, filed on Jul. 26, 2010.

(51) Int. Cl. A61K 31/4545; A61K 31/52; A61K 31/437; A61K 31/496; A61K 31/5377; A61K 31/4709; A61K 31/55; A61K 31/4985

(52) U.S. Cl. CPC A61K 31/4545; A61K 31/55; A61K 31/52; A61K 31/4985; A61K 31/496; A61K 31/5377; A61K 31/4709; A61K 31/437

(57) ABSTRACT

Disclosed is a compound of Formula I:

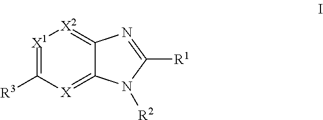

or a salt thereof,
in which X, X¹, X², R¹, R², and R³ are described herein. Also disclosed are pharmaceutical compositions and methods of using the compounds of Formula I to treat disorders mediated by melanocortin-4 receptors.

### TECHNICAL FIELD

The present concerns imidazopyridine, imidazopyrimidine and imidazopyrazine derivatives, their compositions and method of using same to modulate the activity of melanocortin-4 receptors.

### BACKGROUND

Obesity represents the most prevalent of body weight disorders, and it is the most important nutritional disorder in the Western world, with estimates of its prevalence ranging from 30% to 50% of the middle-aged population. The number of overweight and obese Americans has continued to increase since 1960, a trend that is not slowing down. Today, 64.5 percent of adult Americans (about 127 million) are categorized as being overweight or obese. Each year, obesity causes at least 300,000 deaths in the U.S., and healthcare costs of American adults with obesity amount to approximately $100 billion (American Obesity Association).

Obesity increases an individual's risk of developing conditions such as high blood pressure, diabetes (type 2), hyperlipidemia, heart disease, hypertension, stroke, gallbladder disease, and cancer of the breast, prostate, and colon (see, e.g., Nishina et al., 1994, Metabolism. 43: 554-558; Grundy & Barnett 1990, Dis. Mon. 36: 641-731). In the U.S., the incidence of being overweight or obese occurs at higher rates in racial/ethnic minority populations such as African American and Hispanic Americans, compared with Caucasian Americans. Women and persons of low socioeconomic status within minority populations appear to particularly be affected by excess weight and obesity. This trend is not limited to adults. Approximately 30.3 percent of children (ages 6 to 11) are overweight and 15.3 percent are obese. For adolescents (ages 12 to 19), 30.4 percent are overweight and 15.5 percent are obese. Diabetes, hypertension and other obesity-related chronic diseases that are prevalent among adults have now become more common in children and young adults. Poor dietary habits and inactivity are reported to contribute to the increase of obesity in youth. Additionally, risk factors for developing childhood obesity include having overweight parents, or parents unconcerned about their child's weight, increased energy intake due to larger serving sizes, increased sedentary lifestyle and decreased transport-related activity (walking to school or to the bus stop), having a temperament with high levels of anger/frustration (which may cause parents to give their child extra food and calories to decrease tantrums), having Down's Syndrome, mother's pregnancy body mass index (BMI) and first born status (increased prevalence of obesity). One tool used for diagnosing obesity in adults is calculating an individual's BMI, which is a measure of body weight for height (Garrow & Webster, International Journal of Obesity 1985; 9:147-153). A BMI of 25 to 29.9 indicates that an individual is overweight, while a BMI of 30 or above is indicative of obesity. For children, BMI is gender and age specific (Pietrobelli et al., Journal of Pediatrics 1998; 132:204-210). Risk factors for developing obesity in adulthood include poor diet (high-calorie, low nutrients); lack of physical activity; working varied shifts; quitting smoking, having certain medical conditions such as rare hereditary diseases, and hormonal imbalances (such as hypothyroid, Cushing's disease and polycystic ovarian syndrome); certain medications (steroids and some antidepressants); being a racial or ethnic minority (especially a female minority); low socioeconomic status; age (increased risk from 20-55), pregnancy; and retirement (due to altered schedule).

### Melanocortin 4 Receptor and Obesity

Melanocortin (MC) receptors are members of the seven-transmembrane-domain G protein-coupled receptor superfamily that activate generation of the second messenger cyclic AMP (cAMP). There are five MC receptors isolated to date: MC1R, MC2R, MC3R, MC4R and MC5R. Human MC4R is 332 amino acids in length. The melanocortin 4 receptor (MC4R) has been implicated in the regulation of body weight (Graham et al., Nat. Genetics 1997; 17: 273-4). MC4R is expressed in the brain, including the hypothalamus, which influences food intake (Markison & Foster, Drug Discovery Today, 2006, 3, 569).

Signaling via MC4R stimulates anorexigenic neural pathways. MC4R null mice develop late onset obesity with hyperglycemia and hyperinsulinemia. Mice lacking one MC4R allele (heterozygotes) have intermediate body weight between wild-type and homozygous null mice. Transgenic mice overexpressing an endogenous MC4R antagonist, agouti-related protein (AgRP), exhibited increased weight gain, food consumption, and body length compared with non-transgenic littermates (Oilman et al., Science 1997; 278: 135-37). In humans, MC4R deficiency is the most common monogenic form of obesity (Farooqi et al., New Engl. J. Med. 2003; 348: 1085-95). Numerous mutations affecting MC4R activity have been found and many are associated with obesity including early-onset (childhood) obesity (Nijenhuis et al., J. Biol. Chem. 2003, 278:22939-45; Branson et al., New Eng. J. Med. 2003, 348:1096-1103; Gu et al., Diabetes 1999, 48:635-39; Farooqi et al., New Eng. J. Med 2003, 348:1085-95; Tao et al., Endocrinology 2003, 144:4544-51). Pharmacological restoration of mutant melanocortin-4 receptor signaling with cell permeable MC4R ligands has been reported (Ren6 et al., J. Pharmacol. Exp Ther. 2010, 335, 520) Several authors have now reviewed the recent advances in our understanding of the genetics of MC4R in early onset obesity (see e.g., Farooqi & O'Rahilly Int J Obes (Lond), 2005 Oct., 29(10), 1149-52; Govaerts et al., Peptides, 2005 Oct., 26(10), 1909-19; Tao, Mol Cell Endocrinol, 2005 M 15, 239(1-2), 1-14; Farooqi & O'Rahilly, Annu Rev Med, 2005, 56, 443-58). For example, in one patient with severe early-onset obesity, an autosomal-dominant mode of inheritance of an MC4R mutation has been found to be due to a dominant-negative effect caused by receptor dimerization (Biebermann et al., Diabetes, 2003 December, 52(12), 2984-8).

Natural agonists (ligands) of MC4R include [alpha]-MSH, ACTH, [beta]-MSH, and [gamma]-MSH (in order from highest to lowest affinity). Other MC4R ligands, including agonists and antagonists, which have been described to date are peptides (U.S. Pat. No. 6,060,589) and cyclic peptide analogs (U.S. Pat. No. 6,613,874 to Mazur et al.). Further, U.S. Pat. Nos. 6,054,556 and 5,731,408 describe families of agonists and antagonists for MC4R that are lactam heptapeptides having a cyclic structure. A series of MC4R peptide agonists have also been designed (Sun et al., Bioorg Med Chem 2004; 12(10):2671-7). In addition, Nijenhuis et al. (Peptides 2003; 24(2):271-80) described the development and evaluation of melanocortin antagonist compounds that were selective for the MC4R. One compound, designated Ac-Nle-Gly-Lys-D-Phe-Arg-Trp-Gly-NH(2) (SEQ ID NO:9), was found to be the most selective MC4R compound, with a 90- and 110-fold selectivity for the MC4R as compared to the MC3R and MC5R, respectively. Subsequent modification yielded compound Ac-Nle-Gly-Lys-D-Nal(2)-Arg-Trp-Gly-NH(2) (SEQ ID NO: 10), a selective MC4R antagonist with 34-fold MC4R/MC3R and 109-fold MC4R/MC5R selectivity. Both compounds were active in vivo, and crossed the blood-brain barrier. On the other hand, it was recently shown that the moderately selective peptide antagonist PG-932 (7-fold MC4R/MC3R selectivity) increased food intake in mice upon peripheral administration (Sutton et al. Peptides, 2008, 29, 104). A recent report also describes the activation of mutated MC4R by novel peptide agonists (Roubert et al., J. of Endocrinology, 2010, 207, 177).

Other high-affinity MC4R antagonists are described in Grieco et al. J Med Chem 2002; 24:5287-94. These cyclic antagonists were designed based on the known high affinity antagonist SHU9119 (Ac-Nle4-[Asp5-His6-DNal(2′)7-Arg8-Trp9-LyslO]-NH(2)) (SEQ ID NO: 11). The SHU9119 analogues were modified in position 6 (His) with non-conventional amino acids. One compound containing a Che substitution at position 6 is a high affinity MC4R antagonist (IC50=0.48 nM) with 100-fold selectivity over MC3R. Another compound with a Cpe substitution at position 6 also was a high affinity MC4R antagonist (IC50=0.51 nM) with a 200-fold selectivity over MC3R. Molecular modeling was used to examine the conformational properties of the cyclic peptides modified in position 6 with conformationally restricted amino acids. See also, Grieco et al., Peptides 2006; 27(2):472-81. Several non-peptide MC4R ligands have also been disclosed in U.S. published patent applications 2003/0158209 to Dyck et al. and 2004/082590 to Briner et al. Also, U.S. Pat. No. 6,638,927 to Renhowe et al. describes small, low-molecular weight guanidobenzamides as specific MC4R agonists. Richardson et al. have described novel arylpiperizines that are agonists of MC4R (J Med Chem 2004; 47(3):744-55). U.S. Pat. No. 6,979,691 to Yu et al. and U.S. Pat. No. 6,699,873 to Maguire also describe non-peptide compounds which bind selectively to MC4R. WO 99/55679 to Basu et al. discloses isoquinoline derivatives, small molecule non-peptide compounds, which show low (micromolar) affinities for the MC1R and MC4R, reduction of dermal inflammation induced by arachidonic acids, and reductions of body weight and food intake. WO 99/64002 to Nargund et al. also discloses spiropiperidine derivatives as melanocortin receptor agonists, useful for the treatment of diseases and disorders such as obesity, diabetes, and sexual dysfunction. A large number of MC4-receptor ligands developed recently are analogs of N-acylpiperidines or piperazines (Nozawa et al. Expert Opin. Ther. Patents, 2008, 18, 403).

Other non-peptide MC4R antagonists have been described. Thus, U.S. published patent applications 2003/0176425 and 2003/0162819 to Eisinger disclose novel 1,2,4-thiadiazole and 1,2,4-thiadiazolium derivatives, respectively, as MC4R antagonists or agonists. These applications also disclose use of these compounds to treat obesity. Other MC4R binding compounds are described in the following: (DeBoer, Nutrition, 2010, 26, 146). He et al. Bioorg. Med. Chem. Lett. 2010, 20, 6524. Emmerson et al., Curr. Top. Med. Chem. 2007, 7, 1121. Nargund et. al. J. Med. Chem. 2006, 49, 4035. Guo et al. Bioorg. Med. Chem. Lett. 2008; 18, 3242. Sebhat et al. Bioorg. Med. Chem. Lett. 2007; 17, 5720. Chen et al. Bioorg. Med. Chem. 2008; 16, 5606. Marinkovic et al. Bioorg. Med. Chem. Lett. 2008; 18, 4817. Tran et al. Bioorg. Med. Chem. Lett. 2008; 18, 1124. Tran et al. Bioorg. Med. Chem. Lett. 2008; 18, 1931. Bednarek & Fong, Exp Opn Ther Patents 2004; 14: 327-36; Ujjainwalla et al., Bioorg. Med. Chem. Lett. 2005; 15(18):4023-8; WO 10/144344 (Palatin); WO 10/065801 (Palatin); WO 10/037081 (Palatin); WO 10/065802 (Palatin); WO 10/065801 (Palatin); WO 10/065800 (Palatin); WO 10/065799 (Palatin); WO 03/07949 (Merck); WO 10/081666 (Santhera); WO 10/034500 (Santhera); WO 09/080291 (Santhera); WO 09/115321 (Santhera); WO 04/075823 (Ipsen); WO 04/089951 (Ipsen); WO 05/056533 (Ipsen); WO 06/010811 (Ipsen); WO 03/61660 (Eli Lilly); WO 03/09847 (Amgen); WO 03/09850 (Amgen); WO 03/31410 (Neurocrine Biosciences); WO 03/94918 (Neurocrine Biosciences); WO 03/68738 (Neurocrine Biosciences); WO 03/92690 (Procter and Gamble); WO 03/93234 (Procter and Gamble); WO 03/72056 (Chiron); WO 03/66597 (Chiron); WO 03/66587 (Chiron); WO 03/66587 (Chiron); WO 02/67869 (Merck); WO 02/68387 (Merck); WO 02/00259 (Taisho); WO 02/92566 (Taisho); WO 02/070511 (Bristol-Myers Squibb); WO 02/079146 (Bristol-Myers Squibb); WO 10/056022 (LG Life Sciences); Pontillo et al., Bioorg Med Chem Lett. 2005; 15(23):5237-40; Pontillo et al., Bioorg Med Chem Lett. 2005; 15(10):2541-6; Pontillo et al., Bioorg Med Chem Lett. 2004; 14(22):5605-9; Cheung et al., Bioorg Med Chem Lett. 2005; 15(24):5504-8; Yan et al., Bioorg Med Chem Lett. 2004; 15(20); 4611-4; Hsiung et al., Endocrinology. 2005 December; 146(12):5257-66; and Todorovic et al., Peptides. 2005 October; 26(10):2026-36.

### Current Treatments

Current anti-obesity drugs have limited efficacy (Jones, Nat. Rev. Drug Discov. 2009; 8, 834; Yao & Mackenzie, Pharmaceuticals, 2010, 3, 3494)) and numerous side effects (Crowley et al., Nat. Rev. Drug Discov. 2002; 1, 276-86). With obesity reaching epidemic proportions worldwide, there is a pressing need for the development of adequate therapeutics in this area. In recent years, hormones and neuropeptides involved in the regulation of appetite, body energy expenditure, and fat mass accumulation have emerged as potential anti-obesity drugs (McMinn et al., Obes Rev 2000; 1:37-46; Drazen & Woods Curr Opin Clin Nutr Metab Care 2003; 6:621-629). At present, however, these peptides require parenteral administration. The prospect of daily injections to control obesity for extended periods of time (since obesity is a chronic condition) is not very encouraging and limits the use of these drugs.

Thus, there is a need for improved pharmacological agents that are useful to treat obesity in humans.

### BRIEF SUMMARY

Accordingly, there is provided a compound of Formula I:

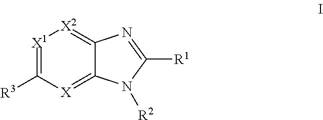

or a salt thereof,
wherein
X is N, and X¹ and X² are both CH; or X¹ is N, and X and X² are both CH; or X² is N, and X and X¹ are both CH; or X and X¹ are both N, and X² is CH or C(OH); or X and X² are both N, and X¹ is CH;

### R¹ is

1) C₁-C₆ alkyl,
2) aryl,
3) heteroaryl,
4) NHR⁴,
5) C₁-C₆ alkyl-aryl,
6) C₁-C₆ alkyl-heteroaryl,
7) C(O) aryl,
8) C(O) heteroaryl,
9) OR⁴,
10) SR⁴, or
11) heterocyclyl
   wherein the aryl, the heteroaryl and the heterocyclyl are optionally substituted with one or more R¹⁰ substituents;

### R² is

1) C₁-C₆ alkyl-heterocyclyl,
2) C₁-C₆ alkyl-N(C₁-C₆ alkyl)₂,
3) C₁-C₆ alkyl-NHC₁-C₆ alkyl,
4) C₁-C₆ alkyl-NH₂, or
5) heterocyclyl,
   wherein the heterocyclyl is optionally substituted with one or more C₁-C₆ alkyl substituents or C₁-C₆ alkyl-aryl;

### R³ is

1) C(O)NR⁵R⁶,
2) CH₂NR⁵R⁶,
3) C(O)-heterocyclyl,
4) heteroaryl,
5) aryl,
6) C(NH)NR⁵R⁶, or
7) CH(CF₃)NR⁵R⁶
   wherein the heteroaryl or the heterocyclyl are optionally substituted with one or more R¹⁰ substituents;

### R⁴ is

1) H,
2) aryl,
3) heteroaryl,
4) C₁-C₆ alkyl,
5) C₁-C₆ alkyl-aryl,
6) C₁-C₆ alkyl-heteroaryl,
7) C₁-C₆ alkyl-NHC(O) C₁-C₆ alkyl,
8) C₁-C₆ alkyl-O—C₁-C₆ alkyl,
9) C₁-C₆ alkyl-heterocyclyl,
10) C₂-C₆ alkenyl,
11) C₃-C₇ cycloalkyl,
12) C(O)-aryl, or
13) C(O)O C₁-C₆ alkyl
   wherein the aryl, the heteroaryl or the heterocyclyl is optionally substituted with one or more R¹⁰ substituents;
   R⁵ and R⁶ are each independently selected from
1) C₁-C₆ alkyl,
2) C₁-C₆ alkyl-C₃-C₇ cycloalkyl,
3) C₁-C₆ alkyl-O—C₁-C₆ alkyl, or
4) C₁-C₆ alkyl-CN;

### R¹⁰ is

1) CN,
2) NO₂,
3) halogen,
4) haloalkyl,
5) C₁-C₆ alkyl,
6) OH,
7) OC₁-C₆ alkyl,
8) O-aryl,
9) C(O)OH,
10) C(O)OC₁-C₆ alkyl,
11) C(O) C₁-C₆ alkyl,
12) C(O) aryl,
13) C(O) heterocyclyl,
14) C(O)NH₂,
15) C(O)NH C₁-C₆ alkyl,
16) C(O)N(C₁-C₆ alkyl)₂,
17) NH₂,
18) NHC(O) C₁-C₆ alkyl,
19) C₁-C₆ alkyl heterocyclyl,
20) C₁-C₆ alkyl (OH),
21) C₁-C₆ alkyl (NH₂),
22) N(C₁-C₆ alkyl)₂,
23) NH(C₁-C₆ alkyl),
24) aryl,
25) heteroaryl, or
26) heterocyclyl,
   wherein the heterocyclyl is optionally substituted with one or more C₁-C₆ alkyl substituents.

According to another aspect, there is provided a pharmaceutical composition comprising a compound of Formula I, according to claim 1, and a pharmaceutically acceptable carrier.

According to another aspect, there is provided a method of treating a disorder mediated by melanocortin-4 receptors, the method comprising administering to a subject in need thereof a therapeutically effective amount of a compound of Formula I, as described above, so as to treat the disorder. In one example, the disorder is obesity, cachexia, eating disorders, diabetes, metabolic diseases, erectile and sexual dysfunction

According to another aspect, there is provided a method of treating obesity in a subject, the method comprising: administering to the subject in need thereof, a pharmaceutically acceptable amount of a compound of Formula I, as described above, so as to treat the obesity.

According to yet another aspect, there is provided a method of modulating melanocortin-4 receptor activity, the method comprising: contacting the receptor with a compound of Formula I, as described above, in an amount sufficient to modulate the receptor activity.

According to one aspect, there is provided an in vitro method of modulating melanocortin-4 receptor activity, the method comprising: contacting a cell with a compound of Formula I, as described above, in an amount sufficient to modulate the receptor activity.

According to one aspect, there is provided use of a compound, as described above, to treat a disorder mediated by melanocortin-4 receptors.

According to another aspect, there is provided use of a compound, as described above, to treat obesity in a subject.

According to yet another aspect, there is provided use of a compound, as described above, in the manufacture of a medicament to treat a disorder mediated by melanocortin-4 receptors.

According to another aspect, there is provided use of a compound, as described above, in the manufacture of a medicament to treat obesity in a subject

### DETAILED DESCRIPTION

### 1) General Overview

We disclose herein imidazo-derivatives that have beneficial pharmaceutical properties and that these compounds may be effective to treat melanocortin-4 mediated diseases such as obesity, cachexia, eating disorders, diabetes, metabolic diseases, erectile dysfunction and/or sexual disorders.

### 2) Compounds

Broadly speaking, the present concerns compounds represented by Formula I:

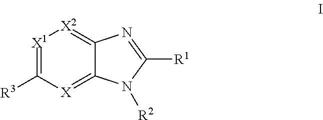

wherein X, X¹, X², R¹, R² and R³ are as defined hereinabove and hereinbelow; or a prodrug or a pharmaceutically acceptable salt to allow the drug to penetrate the cell membrane; or the compound is labeled with a detectable label or an affinity tag thereof.

Further included within the scope of Formula I are compounds of Formula IA, IB, IC, ID, IE and IF:

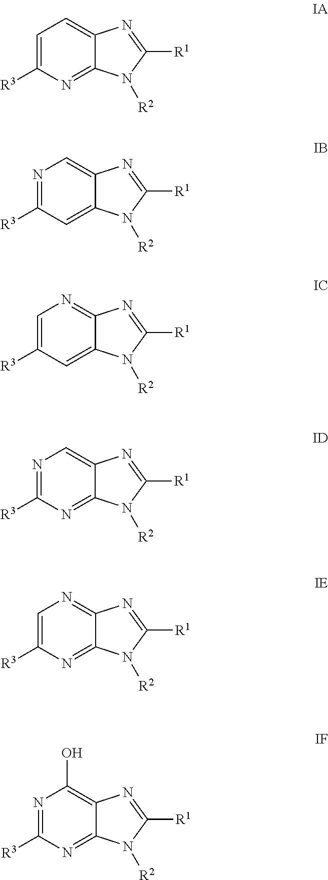

wherein R¹, R² and R³ are as defined hereinabove and hereinbelow; or a prodrug or a pharmaceutically acceptable salt to allow the drug to penetrate the cell membrane; or the compound is labeled with a detectable label or an affinity tag thereof.

Further included within the scope of Formula IA are compounds of Formula IA1 through Formula IA47:

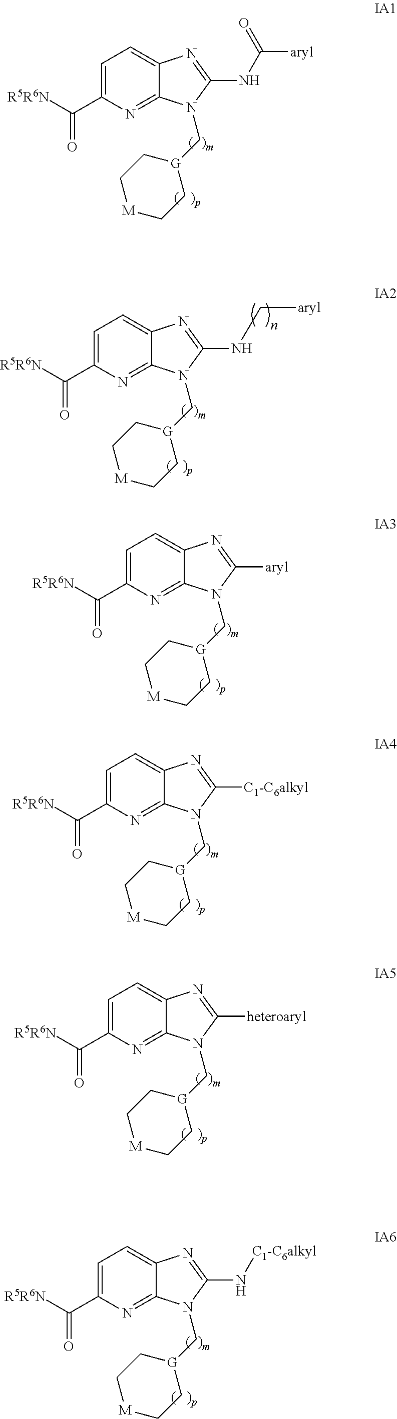

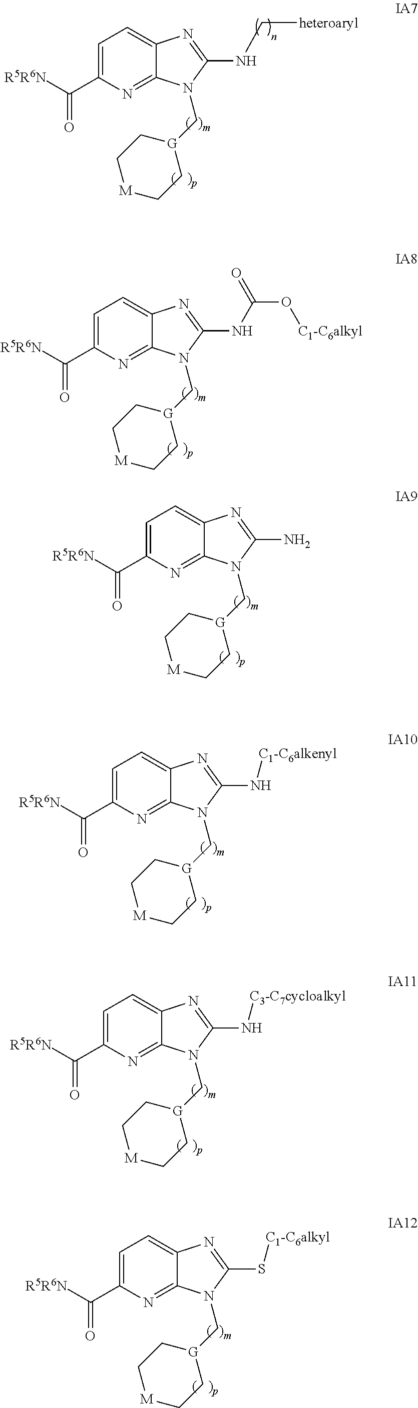

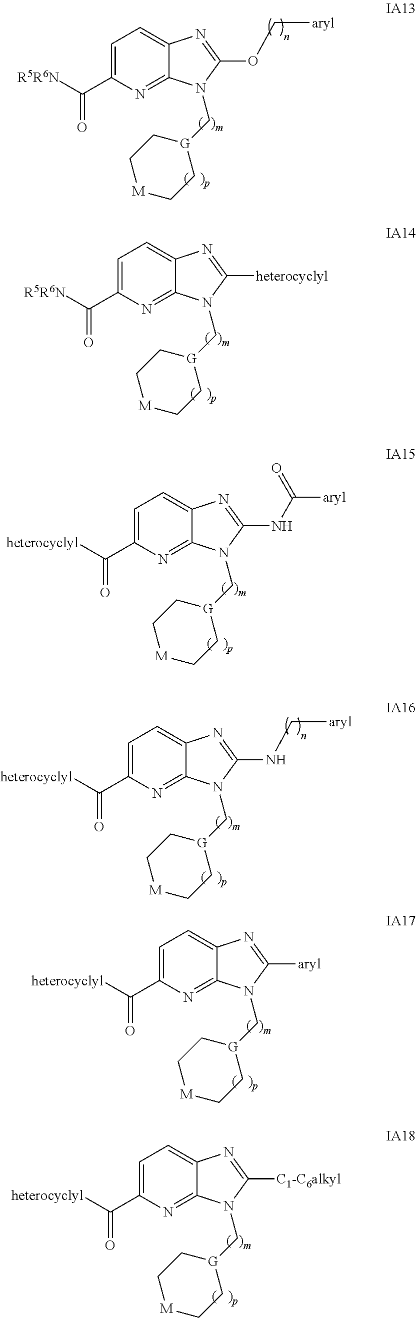

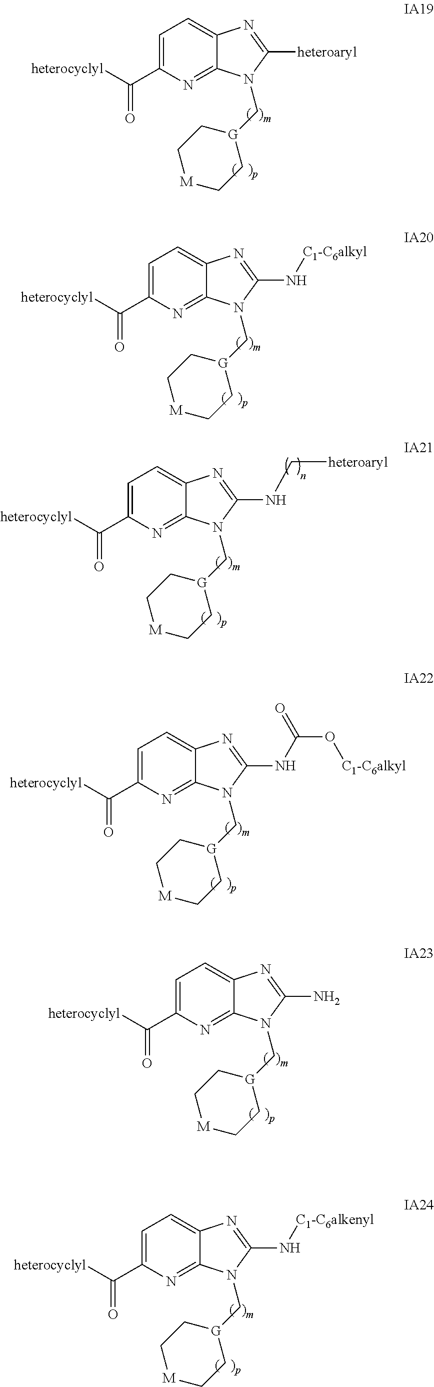

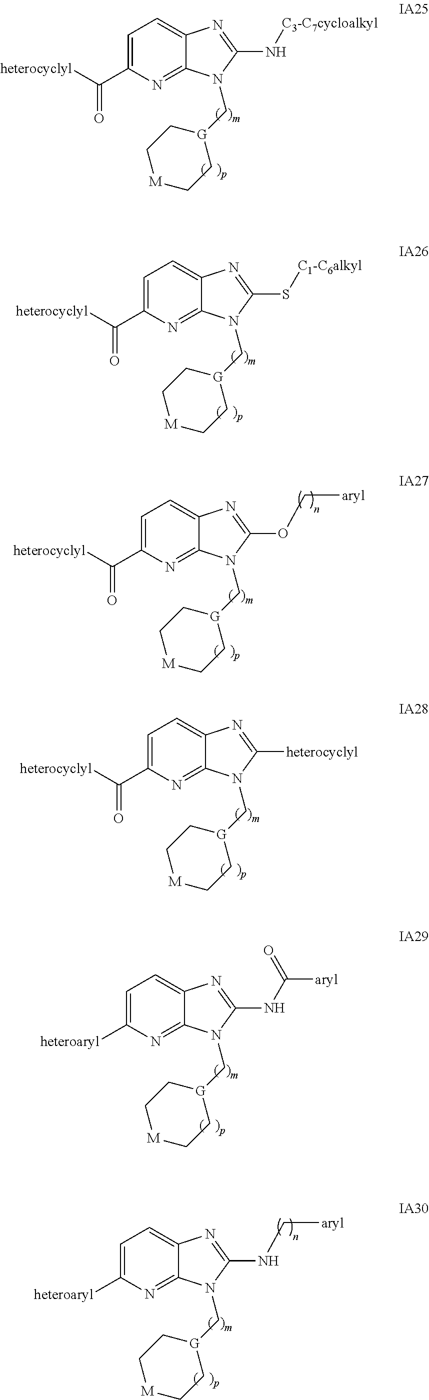

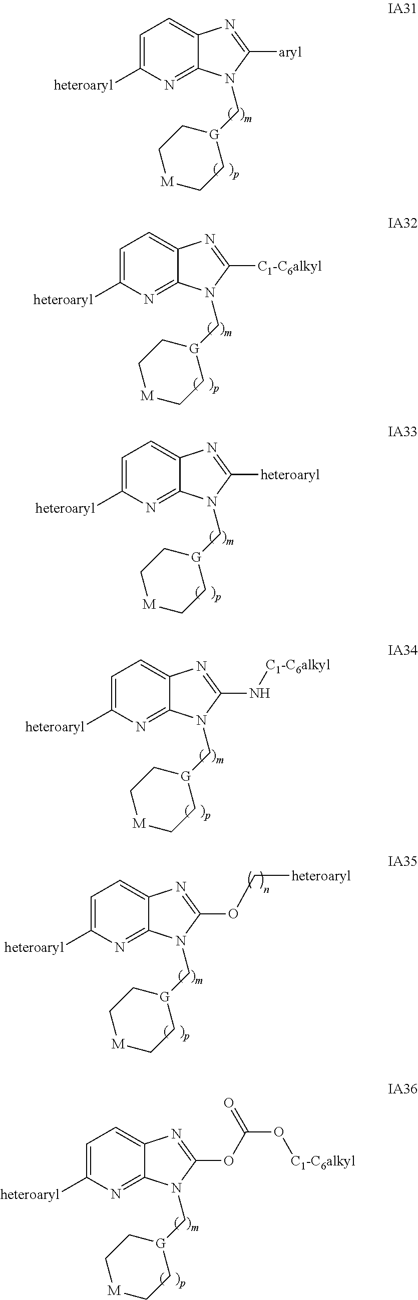

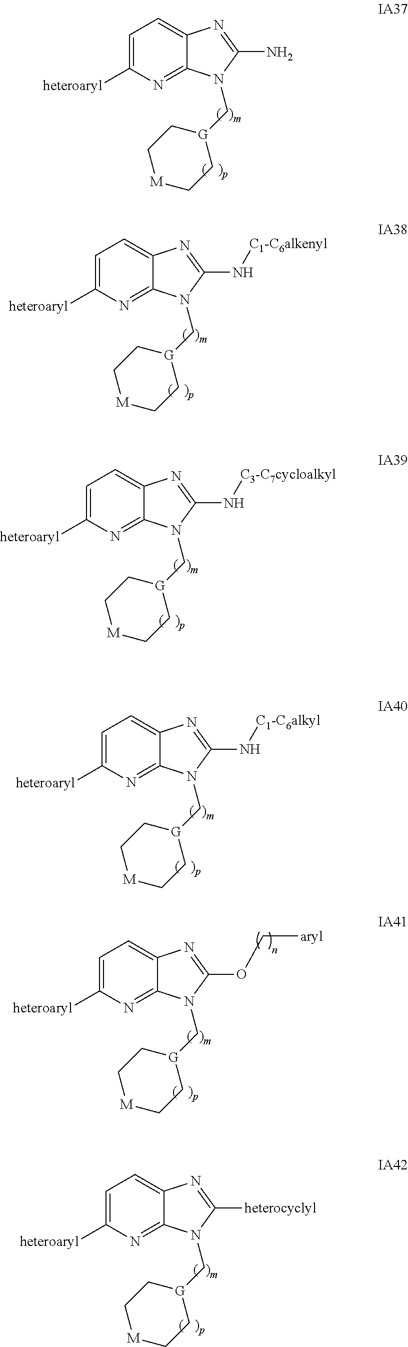

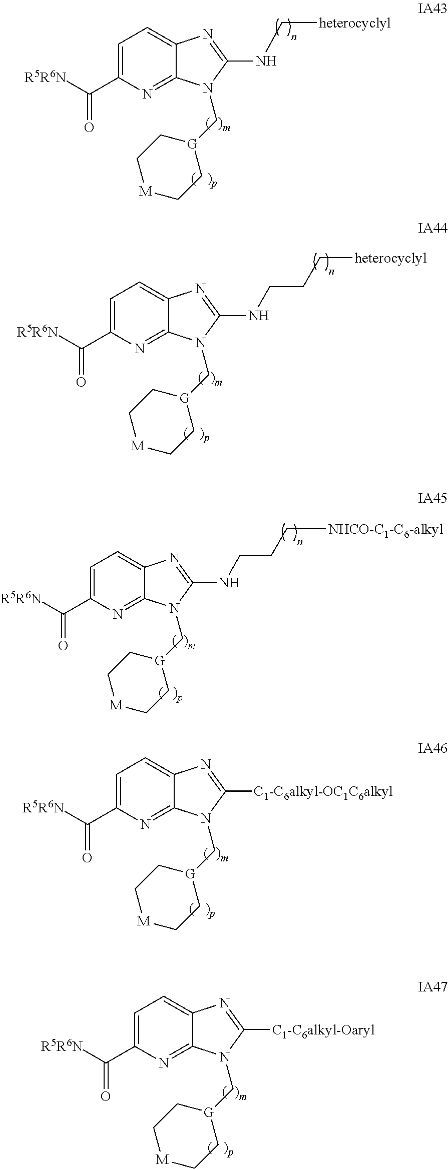

wherein n is an integer from 0 to 1; m is an integer from 0 to 4; p is an integer from 0 to 1; M is CH₂, N, O, or S; G is N, or CH when m is 0; R⁵, R⁶, aryl, heterocyclyl and heteroaryl are as defined hereinabove and hereinbelow; or a prodrug; or a pharmaceutically acceptable salt to allow the drug to penetrate the cell membrane; or the compound is labeled with a detectable label or an affinity tag thereof.

Further included within the scope of Formula IB are compounds of Formula IB1 through Formula IB47:

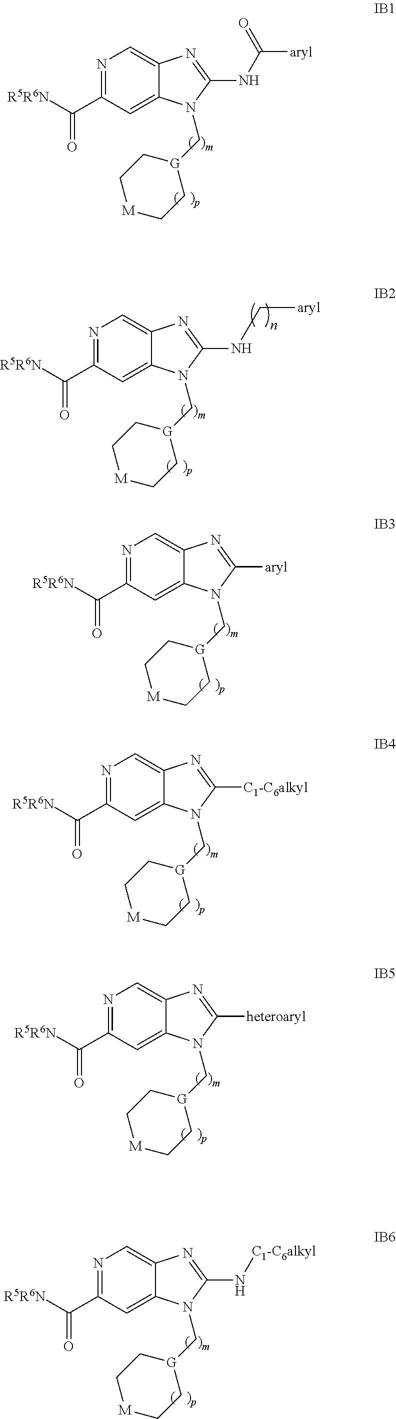

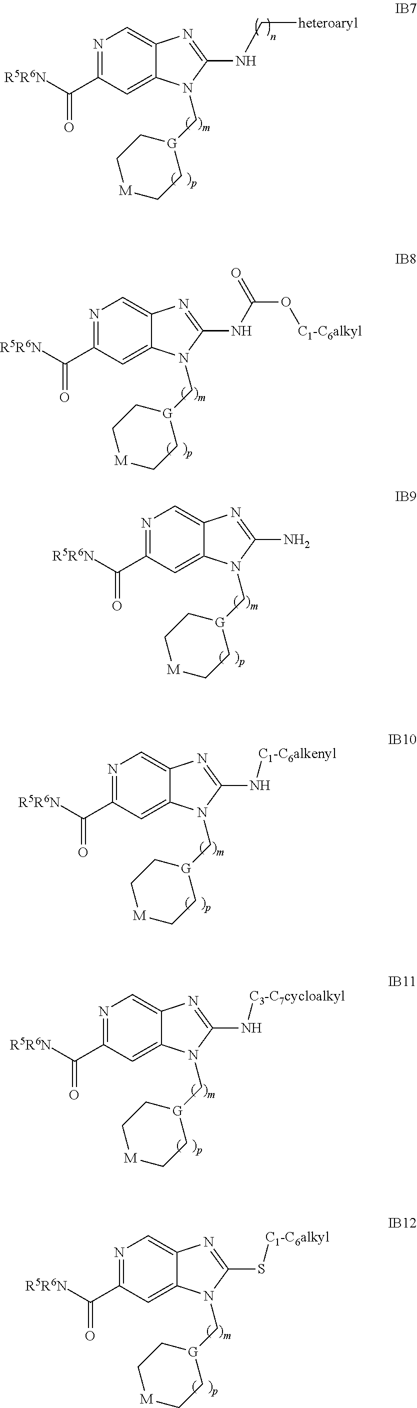

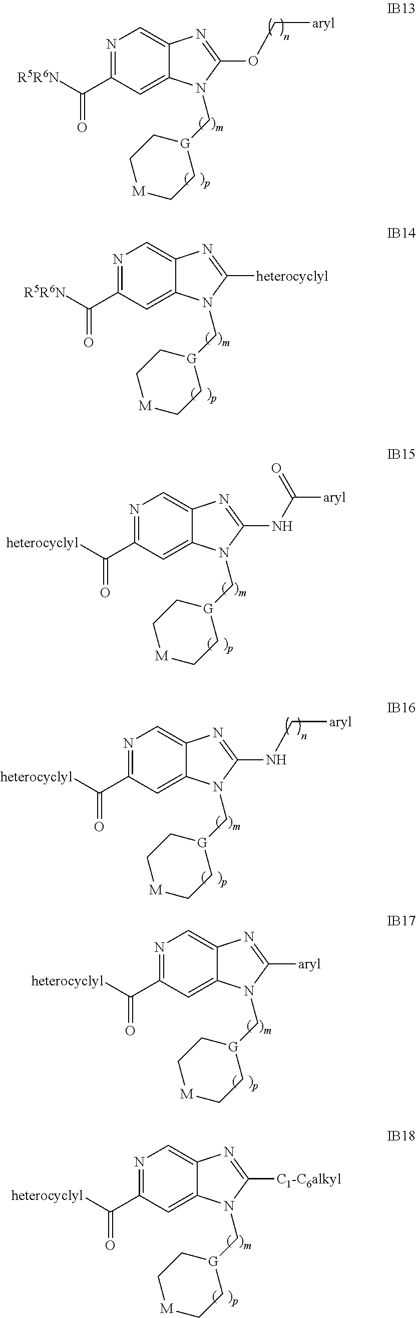

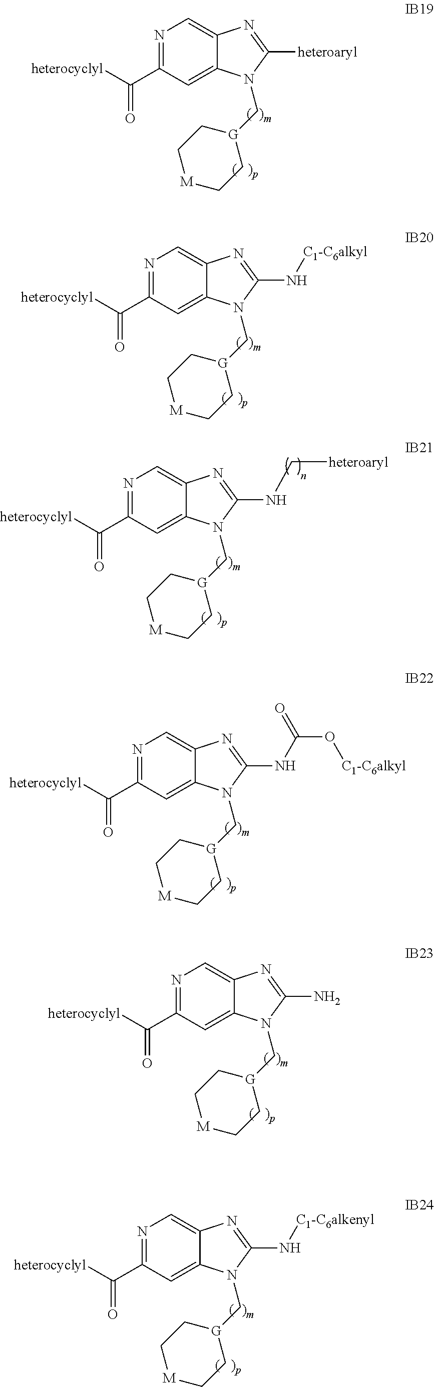

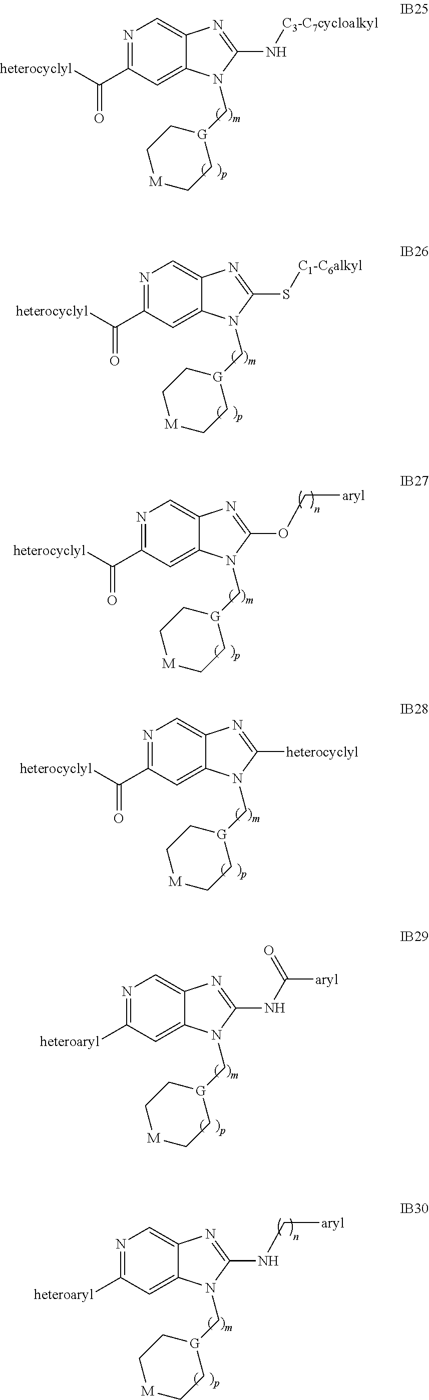

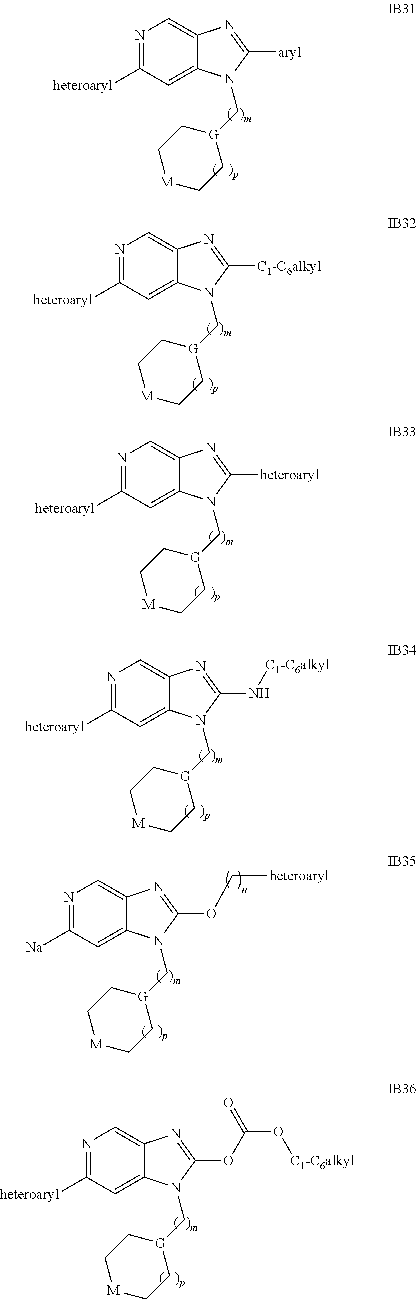

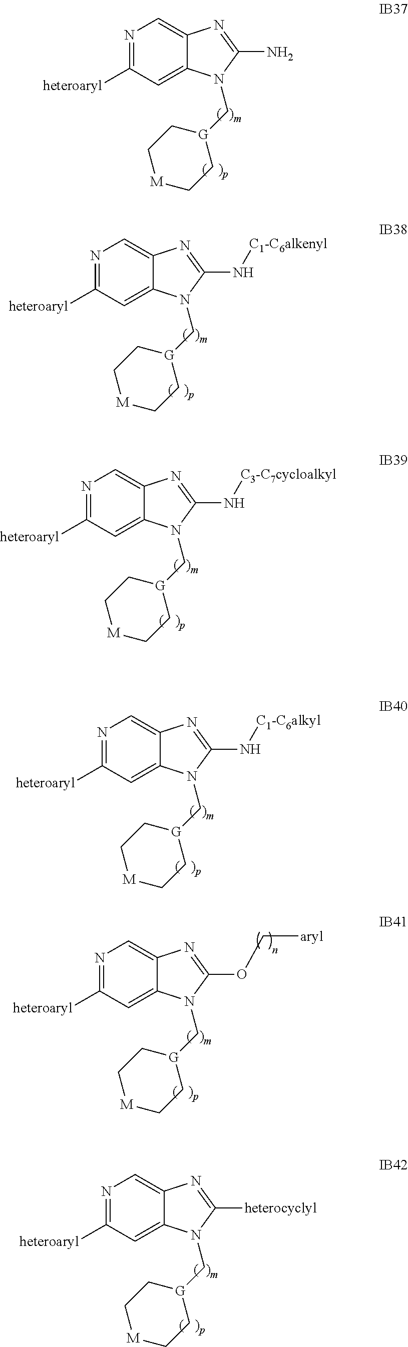

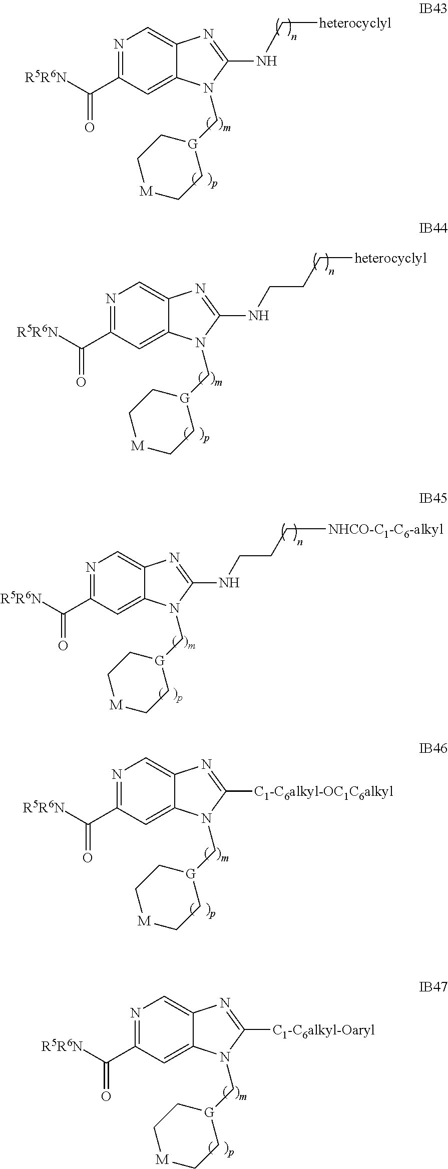

wherein n is an integer from 0 to 1; m is an integer from 0 to 4; p is an integer from 0 to 1; M is CH₂, N, O, or S; G is N, or CH when m is 0; R⁵, R⁶, aryl, heterocyclyl and heteroaryl are as defined hereinabove and hereinbelow; or a prodrug; or a pharmaceutically acceptable salt to allow the drug to penetrate the cell membrane; or the compound is labeled with a detectable label or an affinity tag thereof.

Further included within the scope of Formula IC are compounds of Formula IC1 through Formula IC47:

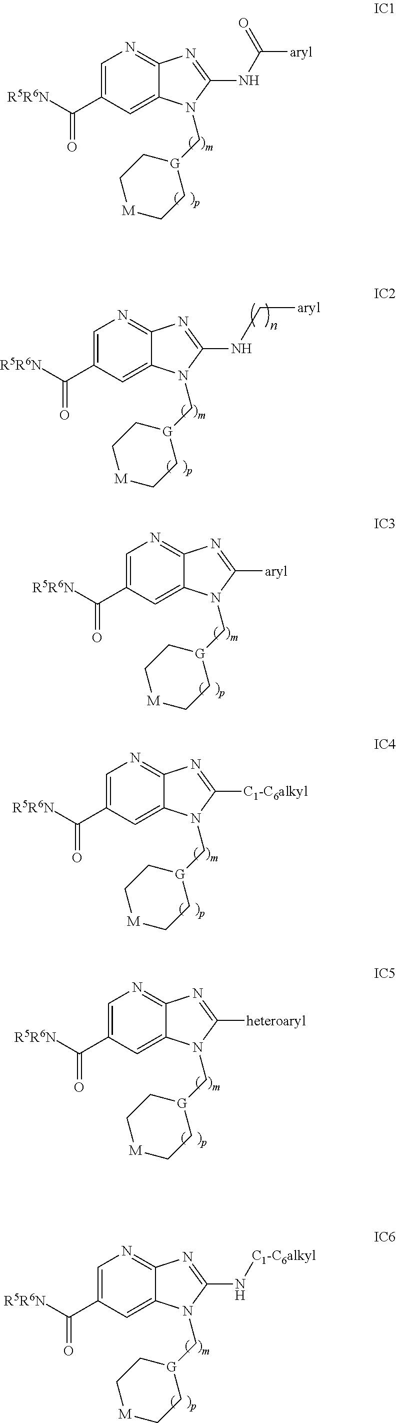

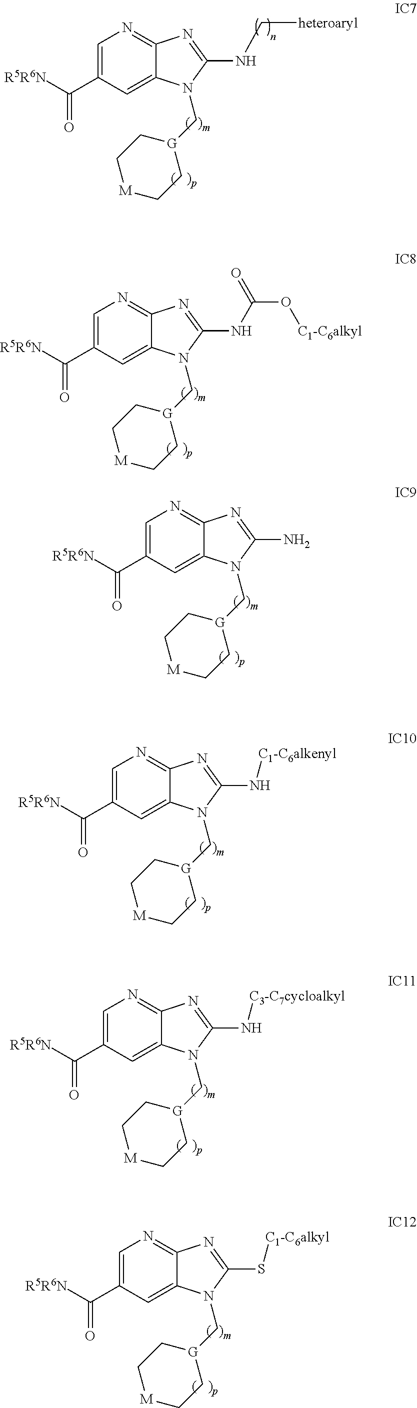

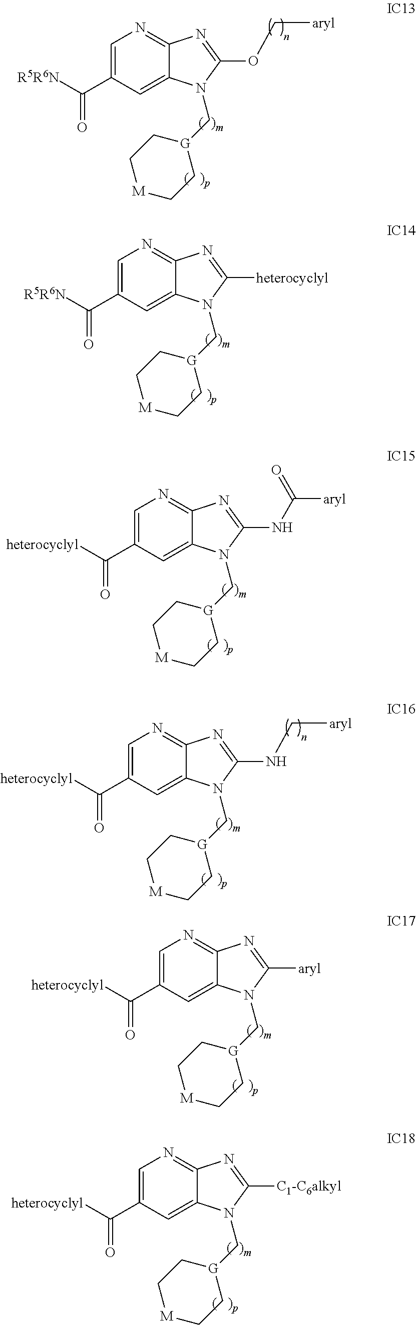

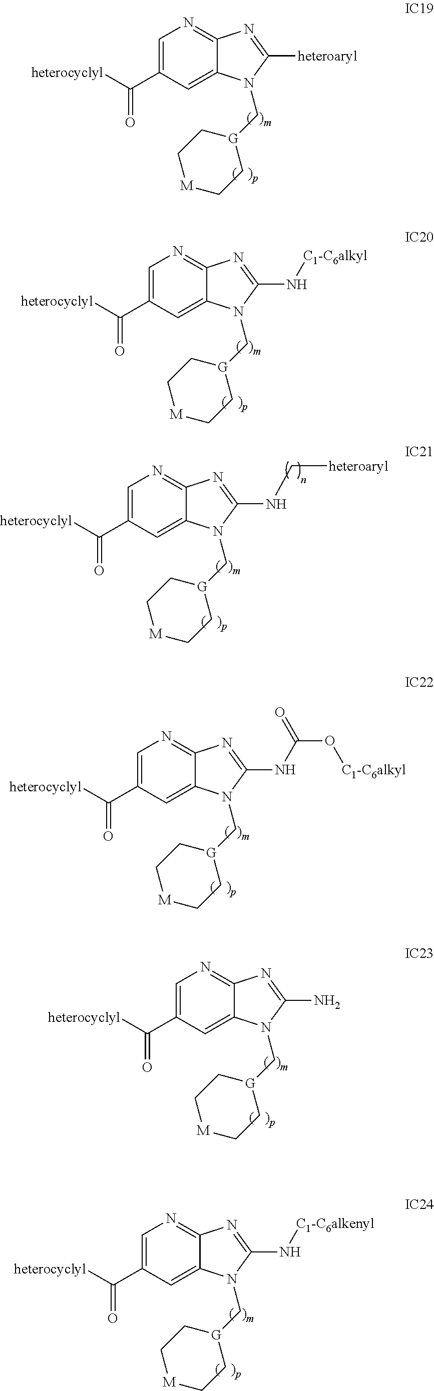

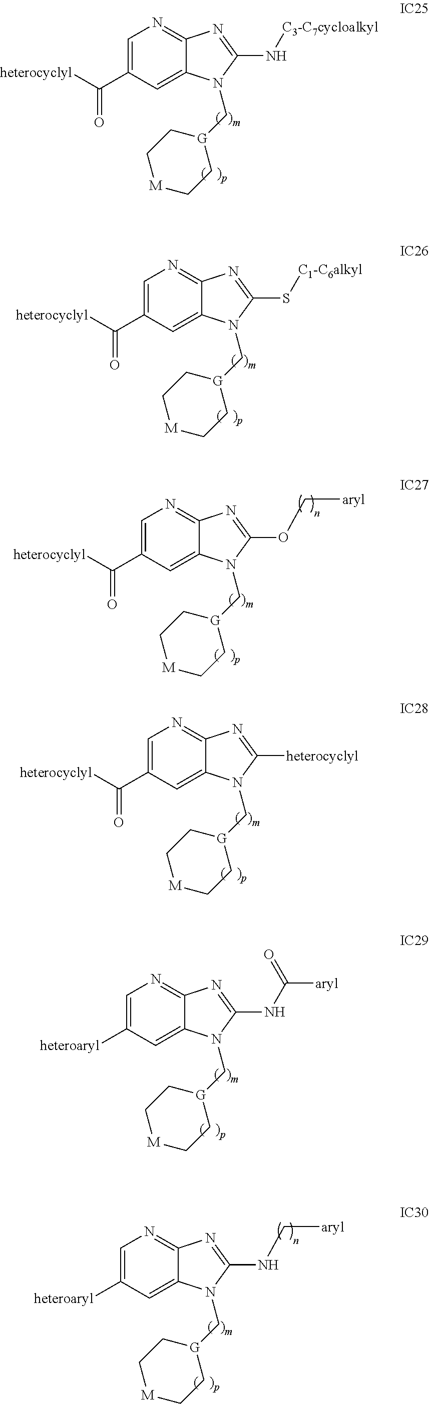

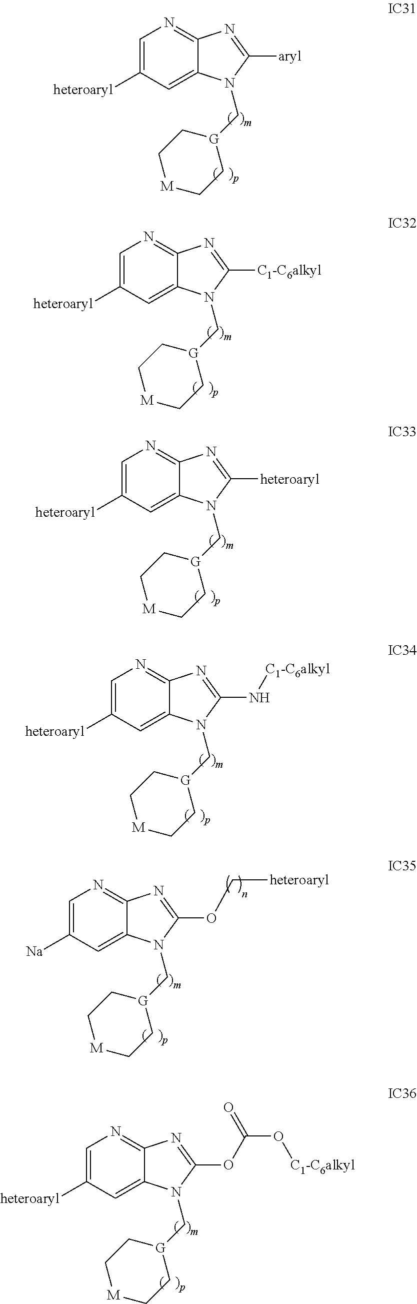

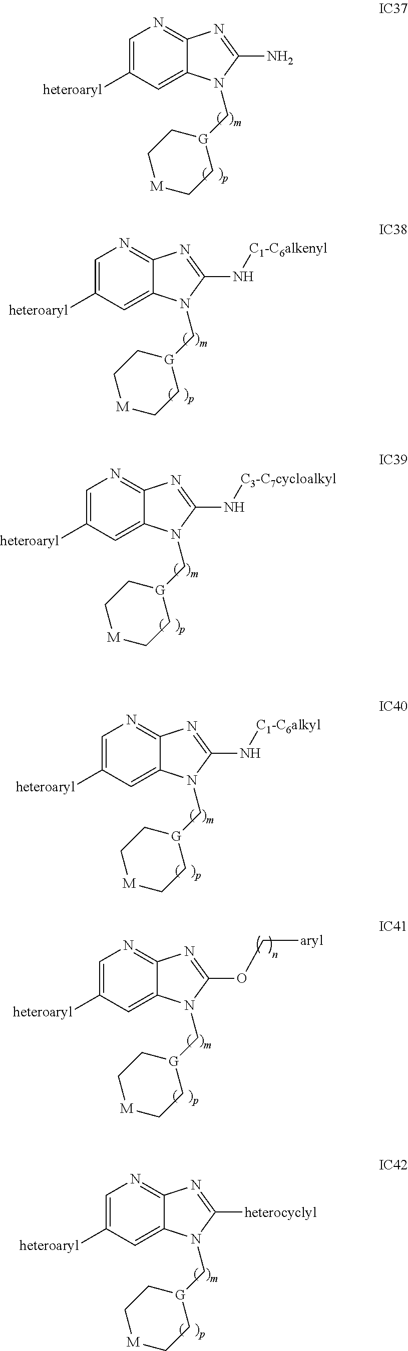

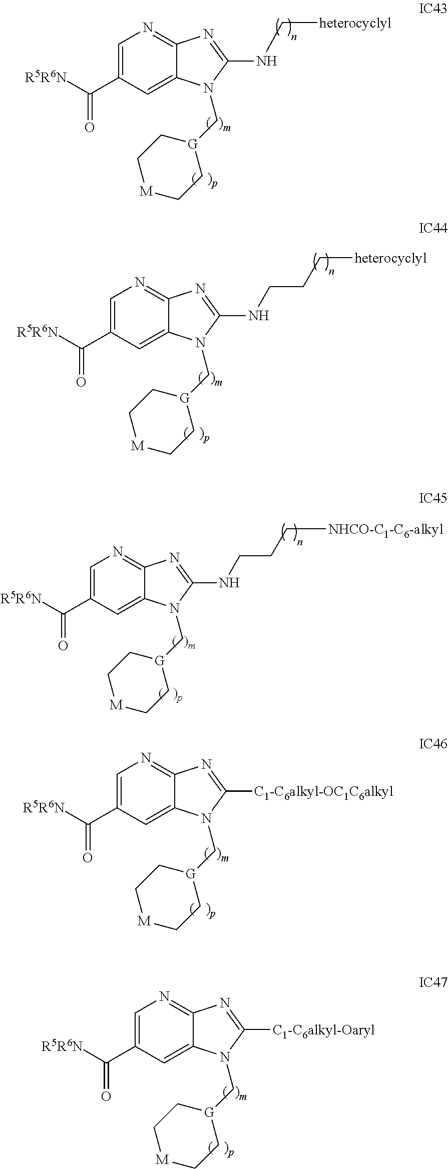

wherein n is an integer from 0 to 1; m is an integer from 0 to 4; p is an integer from 0 to 1; M is CH₂, NH, NC₁-C₆ alkyl-aryl, O, or S; G is N, or CH when m is 0; R⁵, R⁶, aryl, heterocyclyl and heteroaryl are as defined hereinabove and hereinbelow; or a prodrug; or a pharmaceutically acceptable salt to allow the drug to penetrate the cell membrane; or the compound is labeled with a detectable label or an affinity tag thereof.

Further included within the scope of Formula ID are compounds of Formula ID1 through Formula ID47:

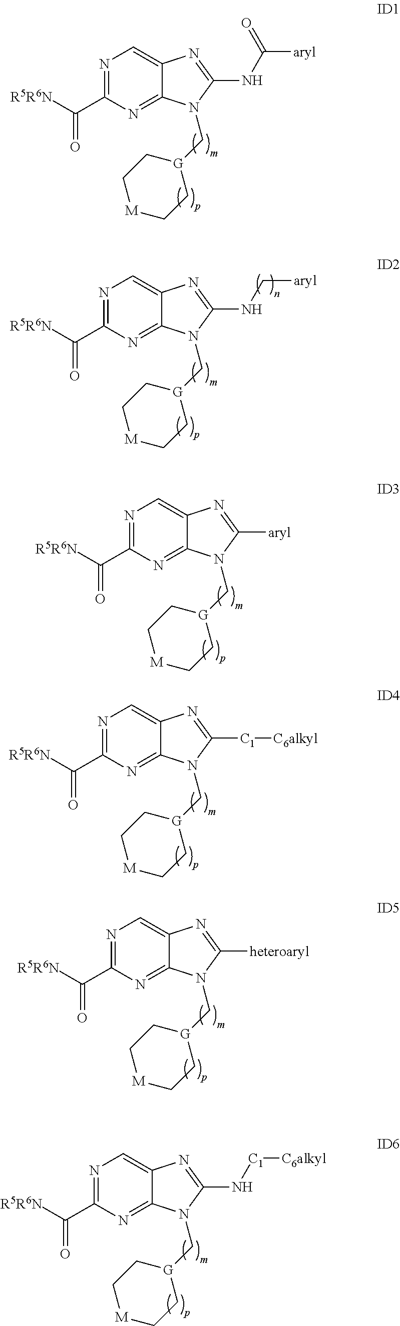

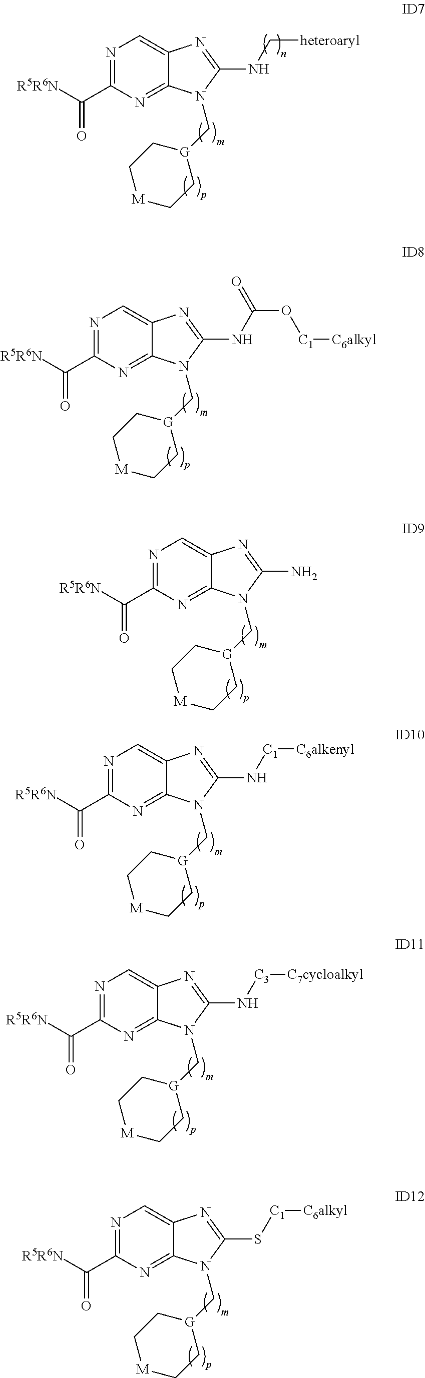

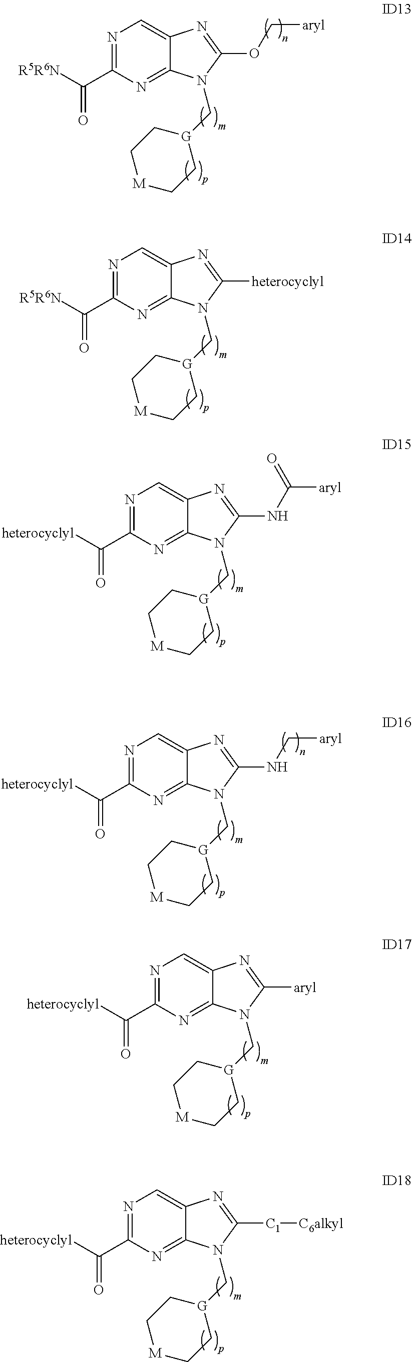

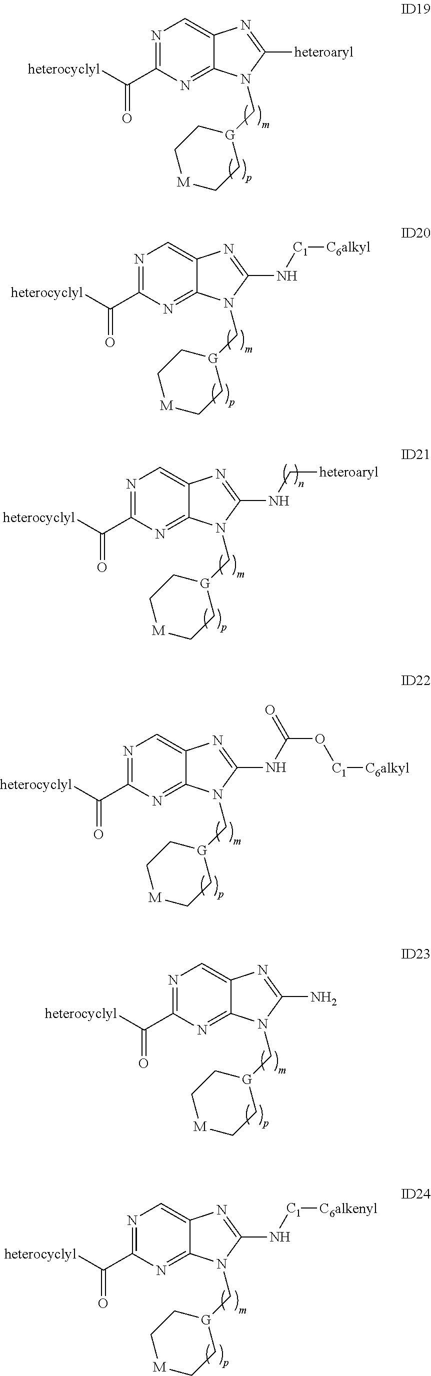

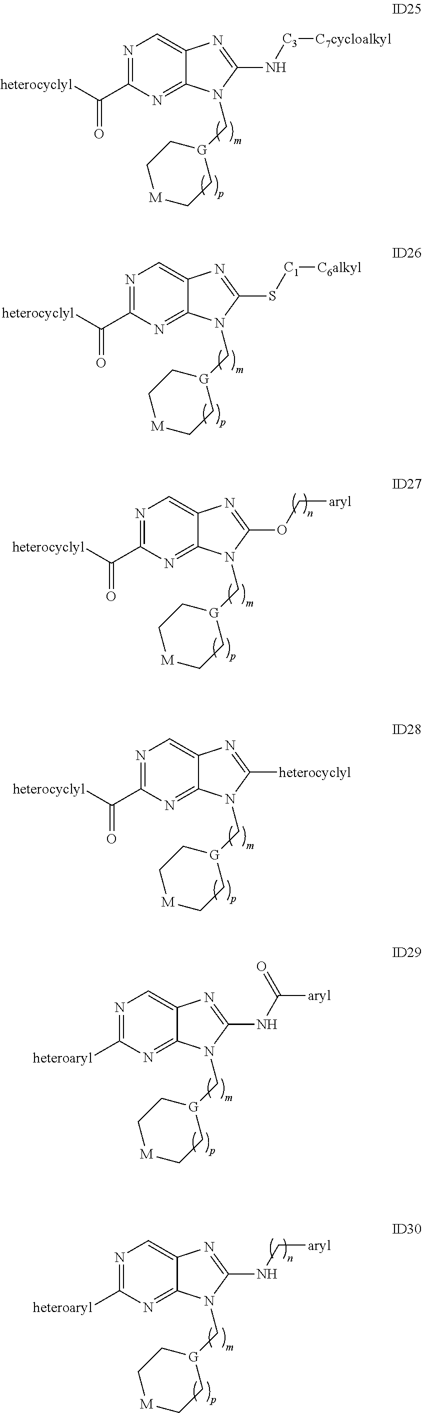

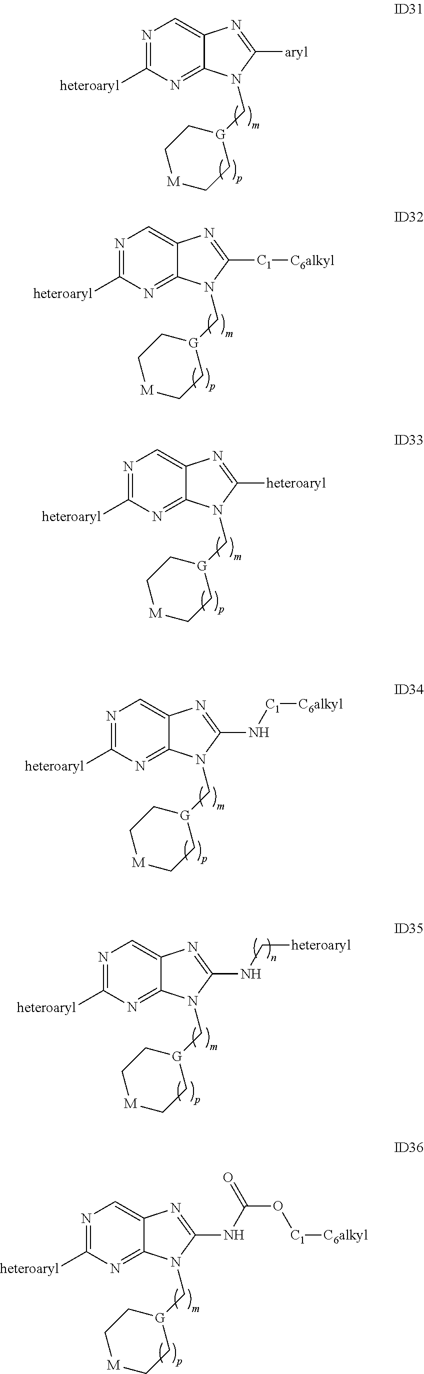

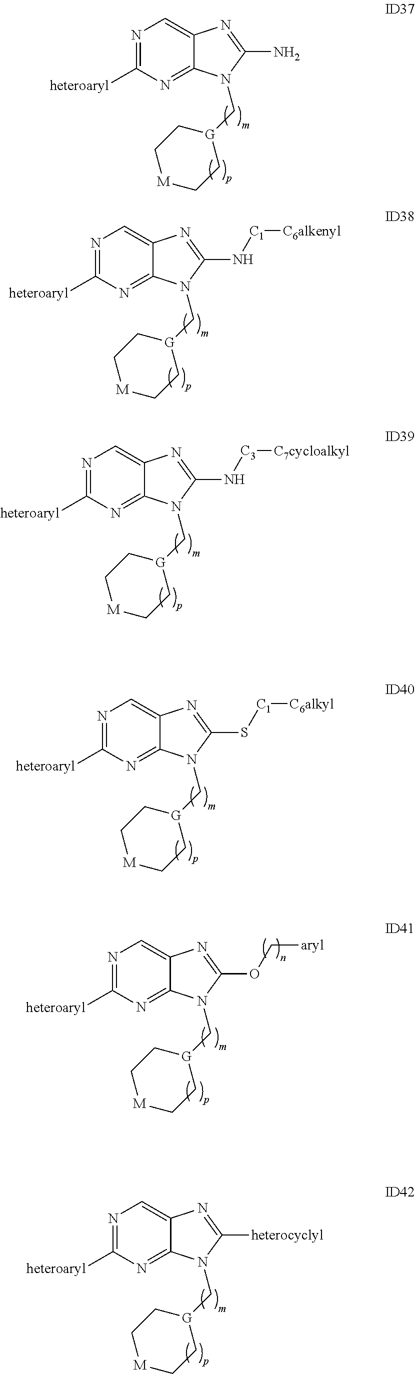

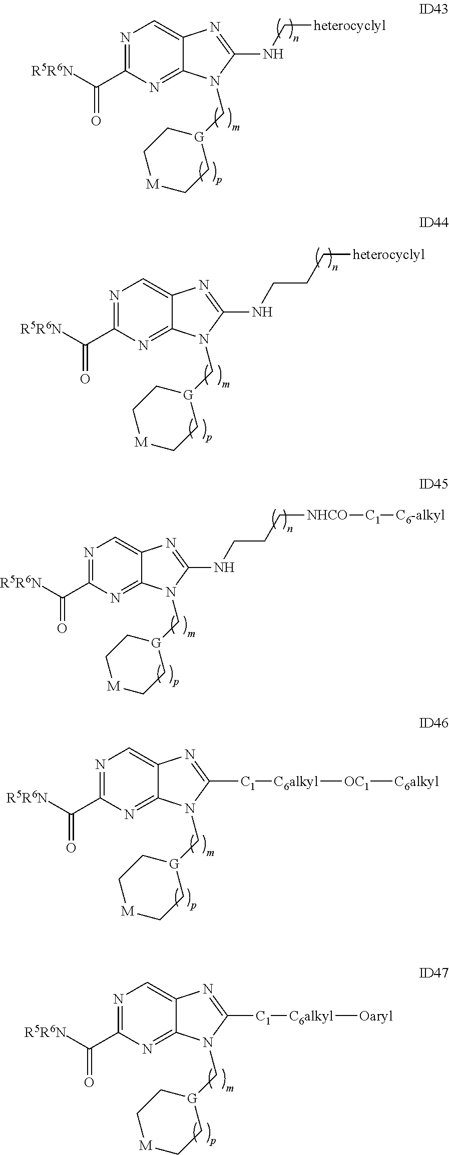

wherein n is an integer from 0 to 1; m is an integer from 0 to 4; p is an integer from 0 to 1; M is CH₂, NH, NC₁-C₆ alkyl-aryl, O, or S; G is N, or CH when m is 0; R⁵, R⁶, aryl, heterocyclyl and heteroaryl are as defined hereinabove and hereinbelow; or a prodrug; or a pharmaceutically acceptable salt to allow the drug to penetrate the cell membrane; or the compound is labeled with a detectable label or an affinity tag thereof.

Further included within the scope of Formula IE are compounds of Formula IE1 through Formula IE47:

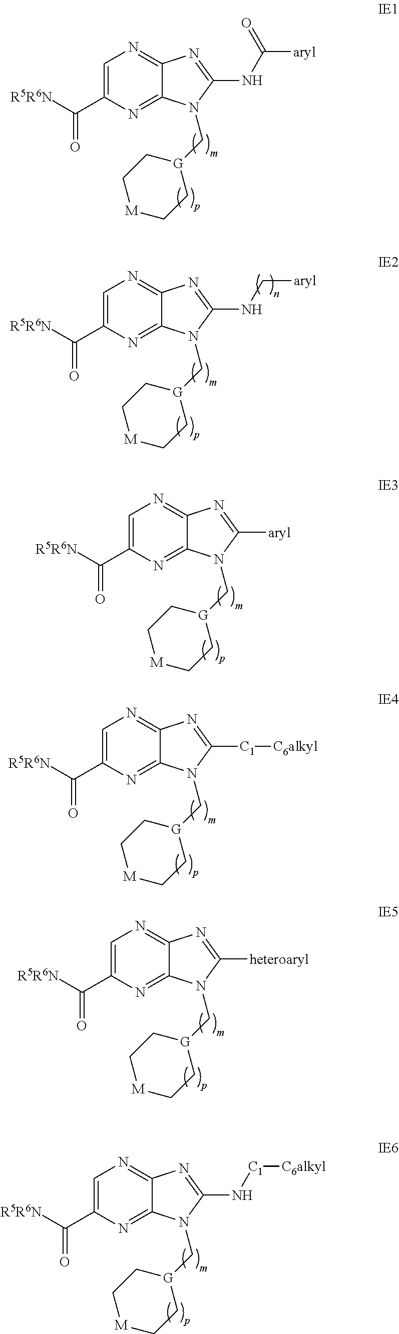

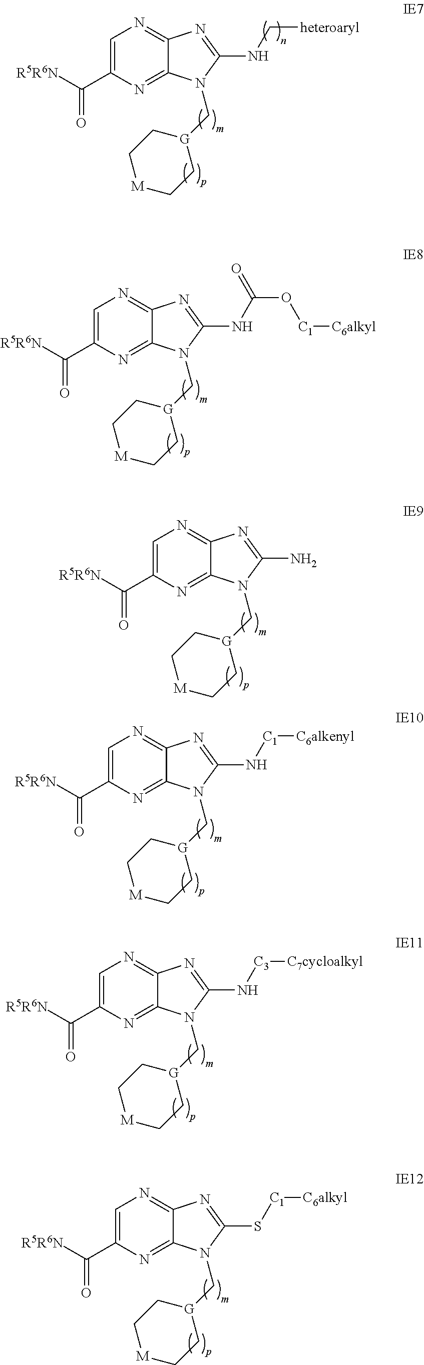

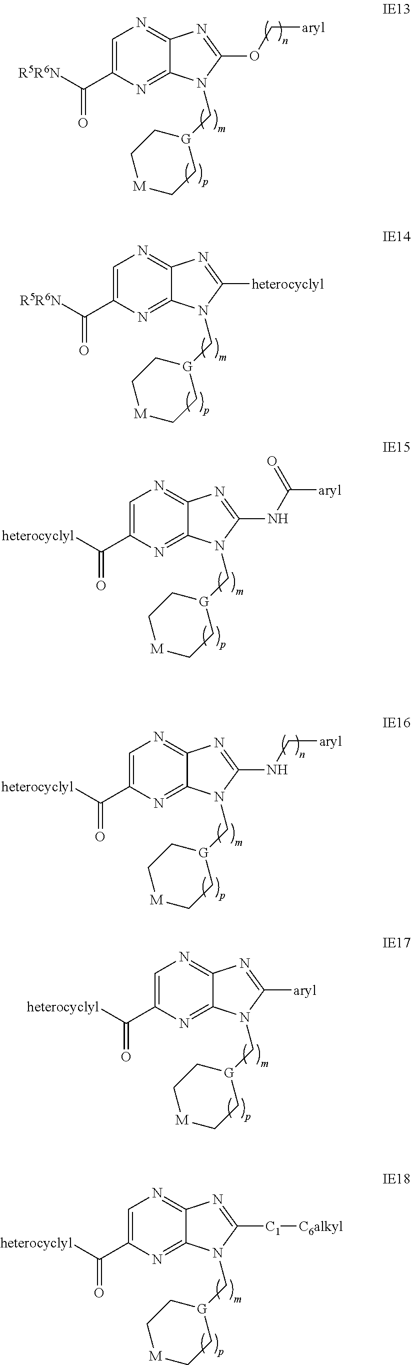

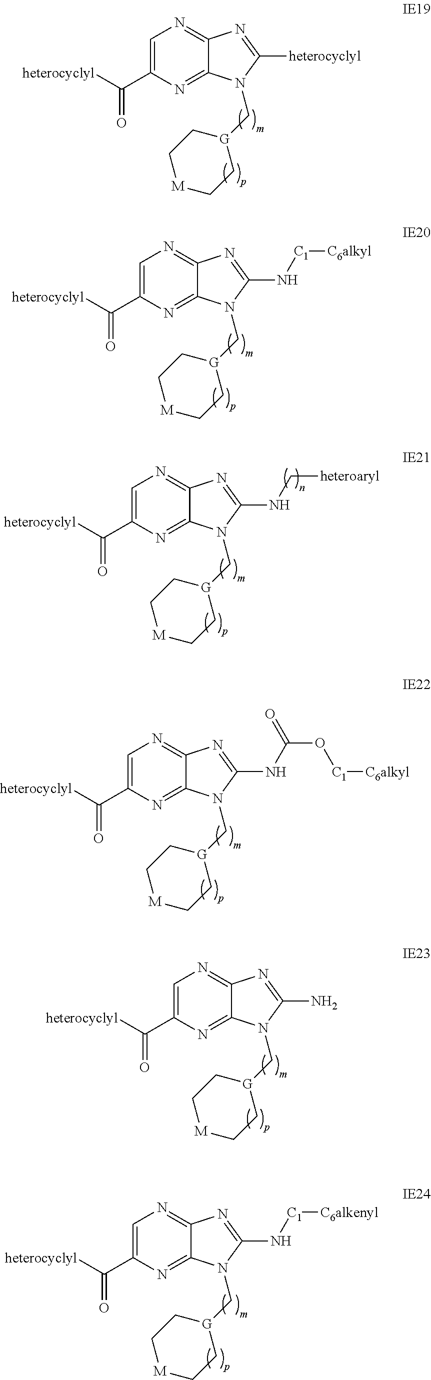

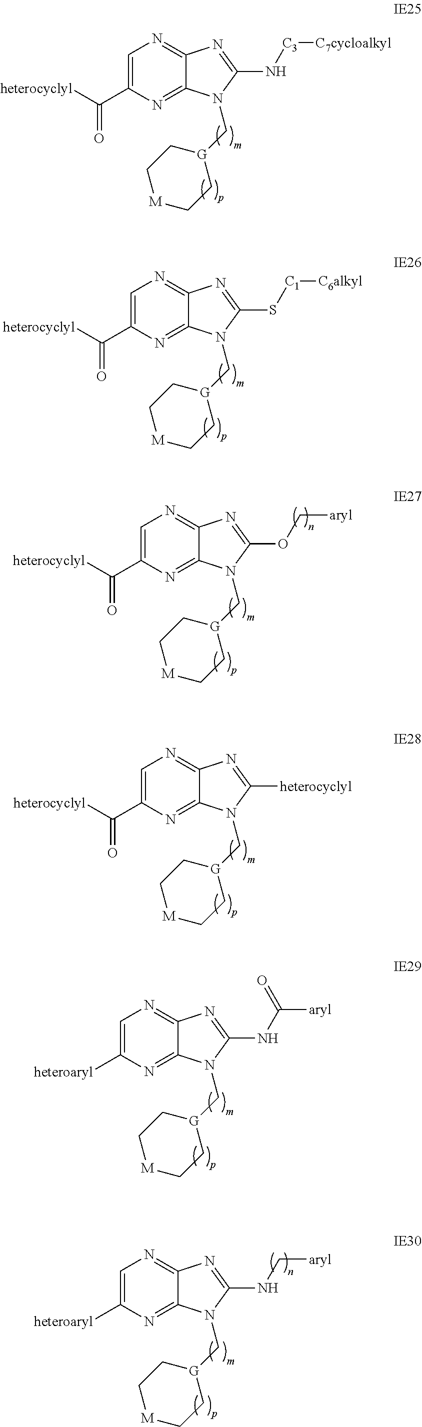

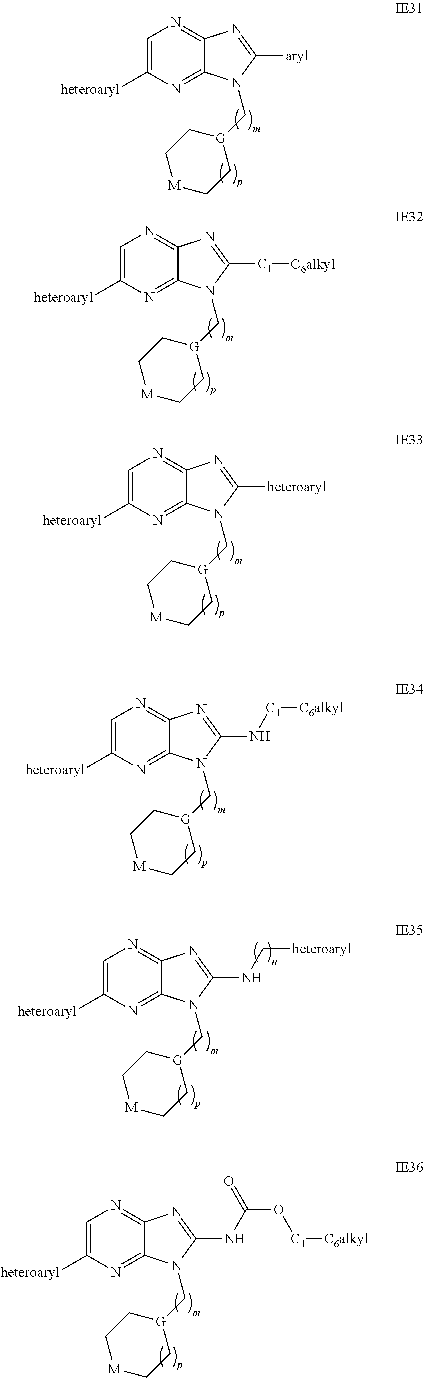

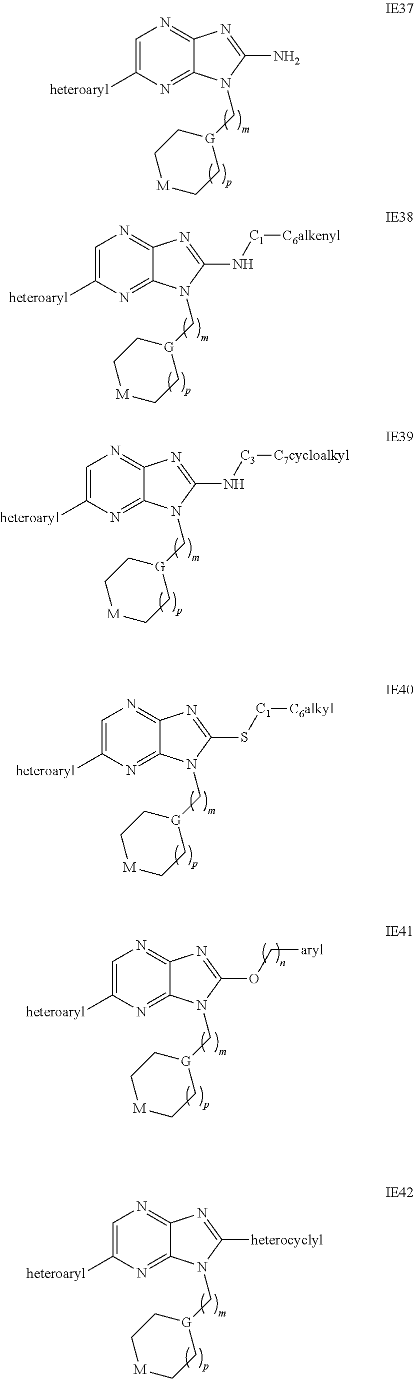

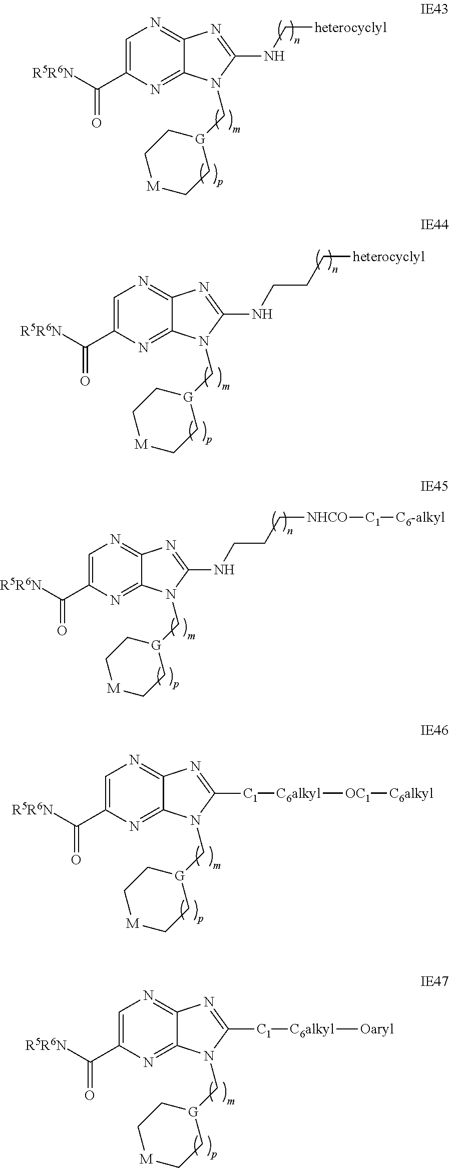

wherein n is an integer from 0 to 1; m is an integer from 0 to 4; p is an integer from 0 to 1; M is CH₂, NH, NC₁-C₆ alkyl-aryl, O, or S; G is N, or CH when m is 0; R⁵, R⁶, aryl, heterocyclyl and heteroaryl are as defined hereinabove and hereinbelow; or a prodrug; or a pharmaceutically acceptable salt to allow the drug to penetrate the cell membrane; or the compound is labeled with a detectable label or an affinity tag thereof.

Further included within the scope of Formula IF are compounds of Formula IF1 through Formula IF47:

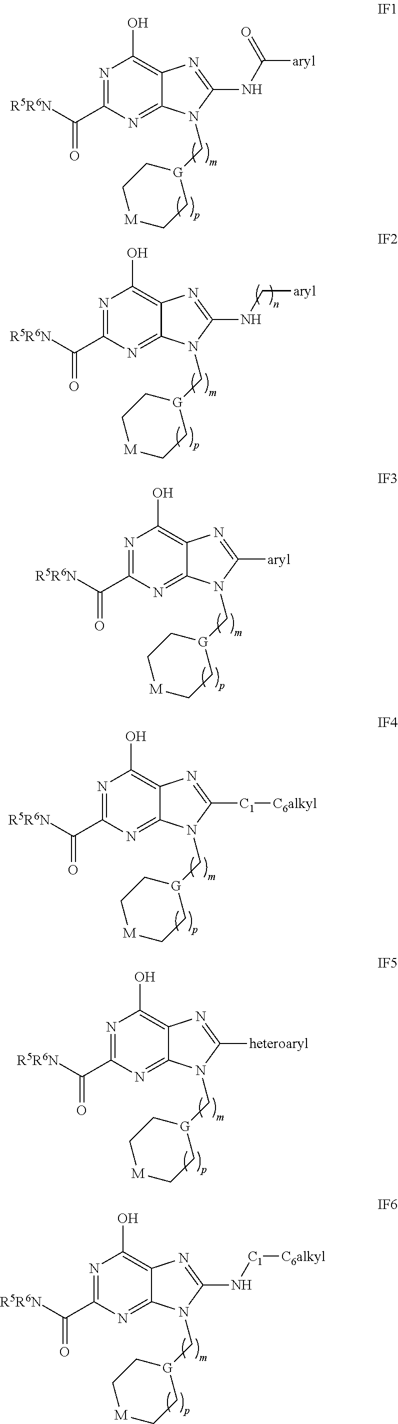

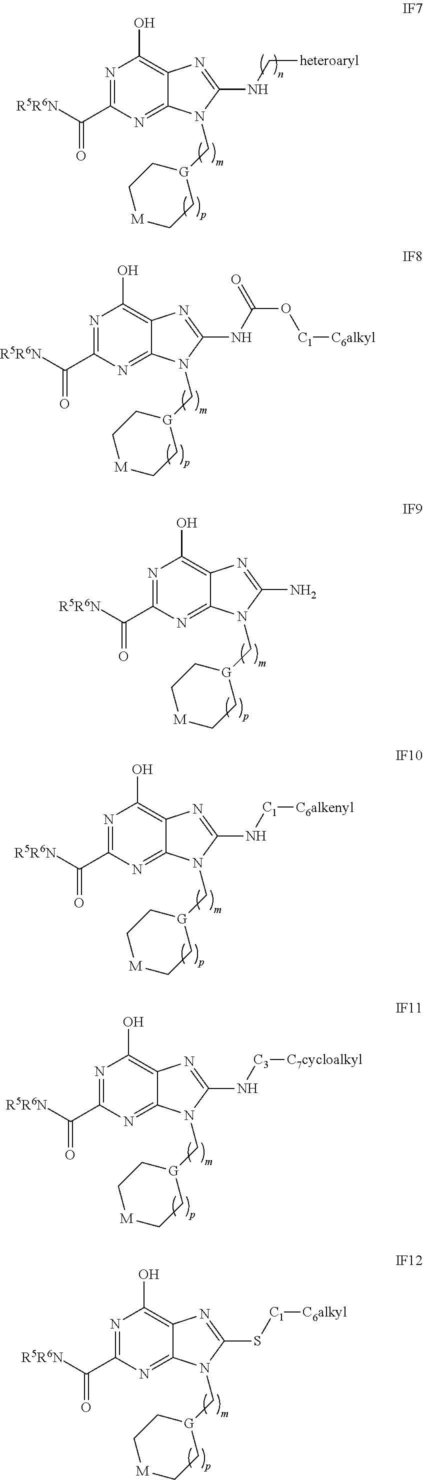

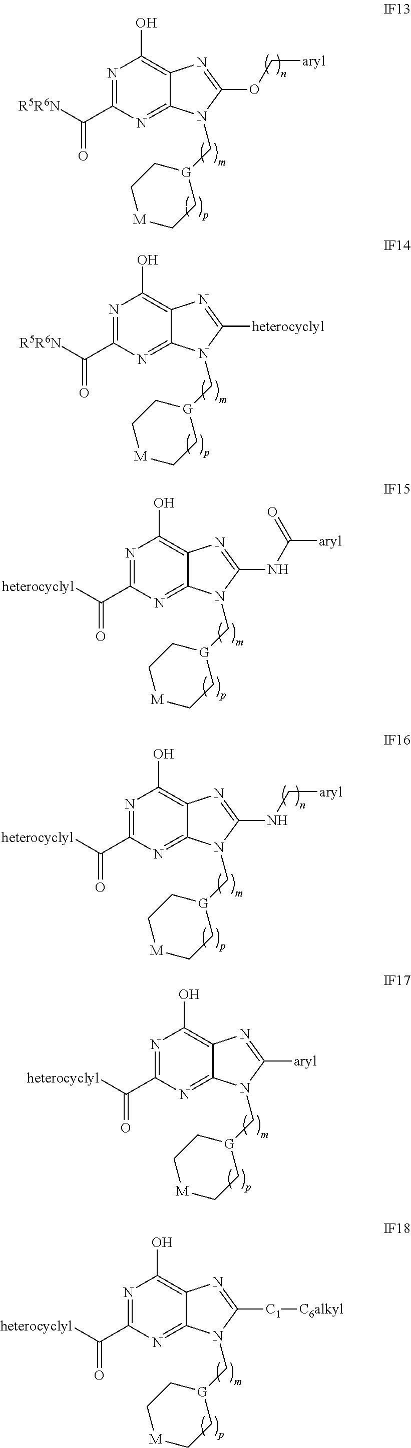

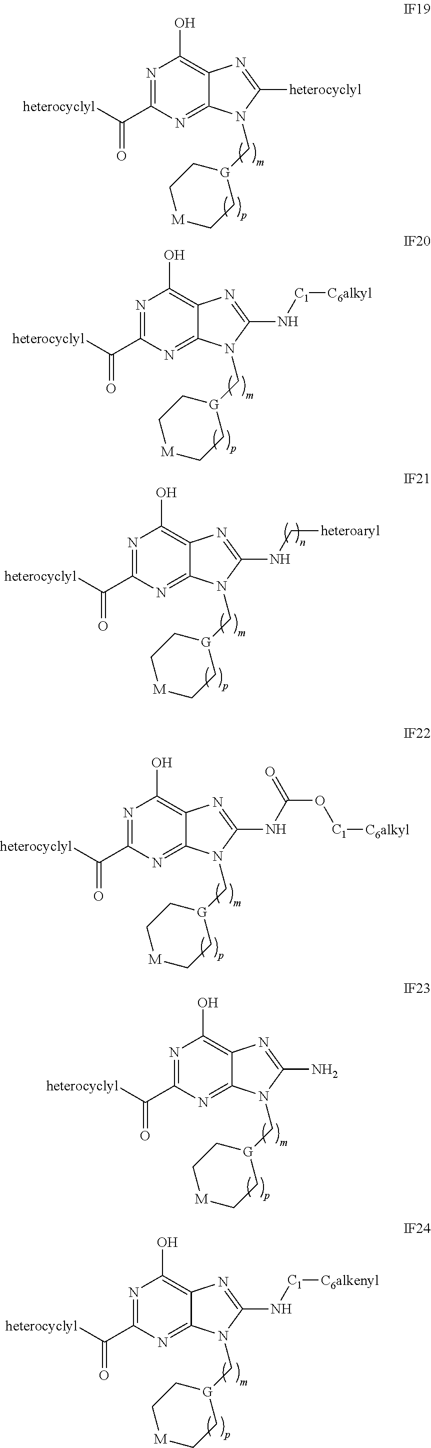

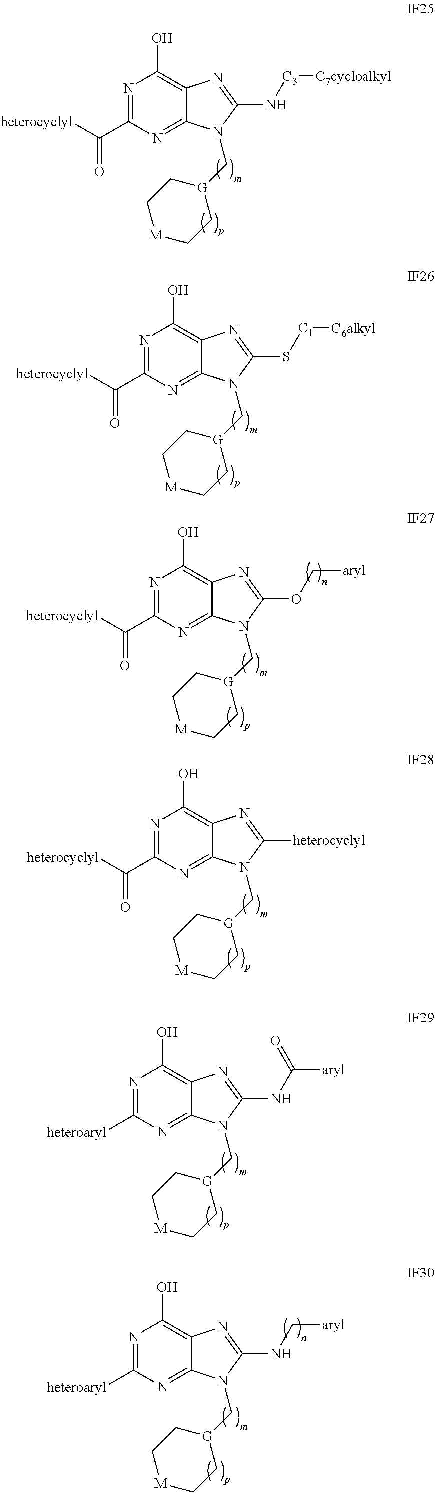

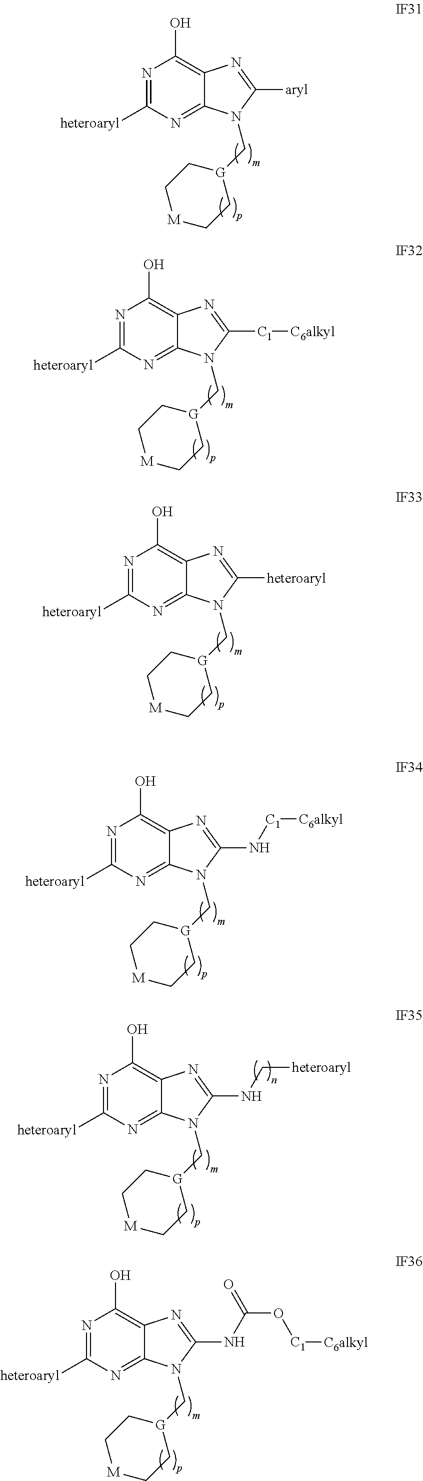

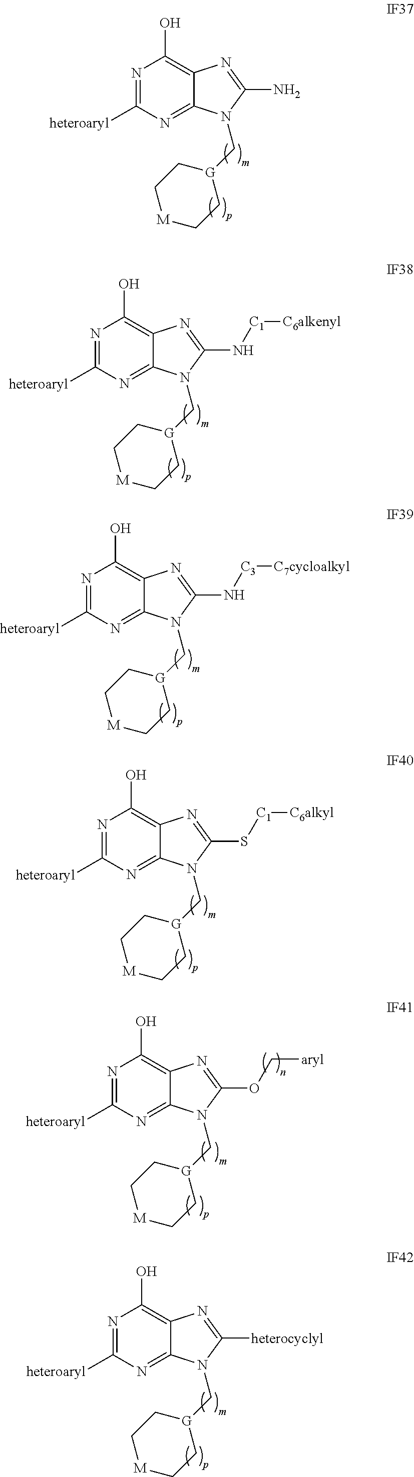

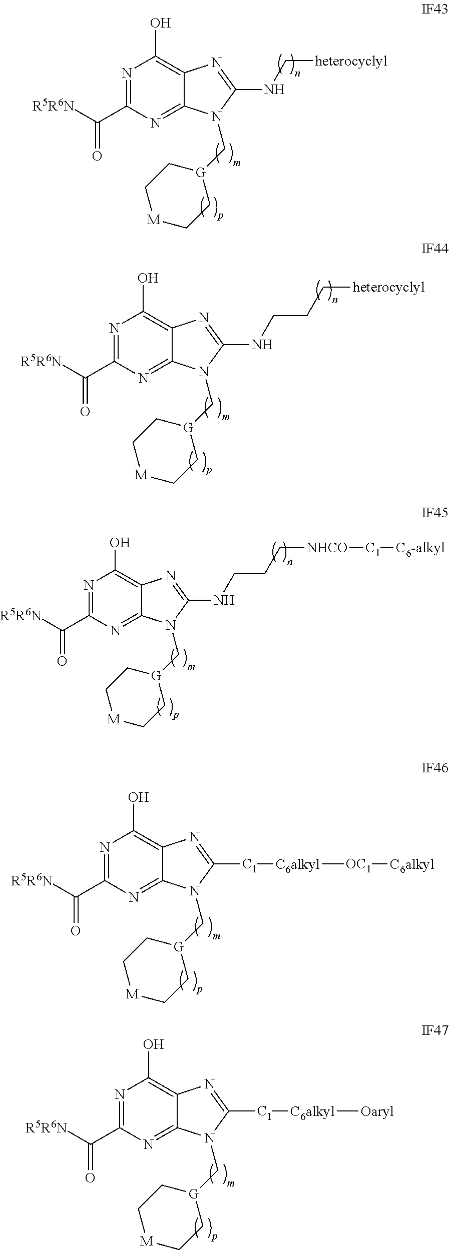

wherein n is an integer from 0 to 1; m is an integer from 0 to 4; p is an integer from 0 to 1; M is CH₂, NH, NC₁-C₆ alkyl-aryl, O, or S; G is N, or CH when m is 0; R⁵, R⁶, aryl, heterocyclyl and heteroaryl are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula IA1, there is provided a compound of Formula IA1.1

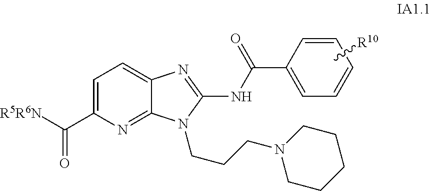

wherein R⁵, R⁶, and R¹⁰ are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula IA2, there is provided a compound of Formula IA2.1

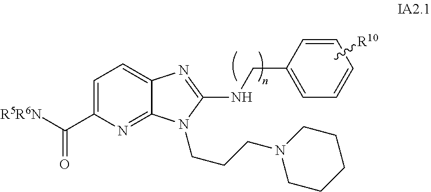

wherein n is an integer from 0 to 1; R⁵, R⁶, and R¹⁰ are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula IA3, there is provided a compound of Formula IA3.1

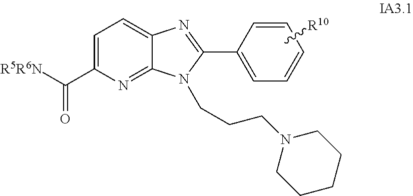

wherein R⁵, R⁶, and R¹⁰ are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula IA4, there is provided a compound of Formula IA4.1

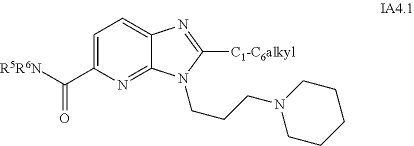

wherein R⁵ and R⁶ are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula IA5, there is provided a compound of Formula IA5.1

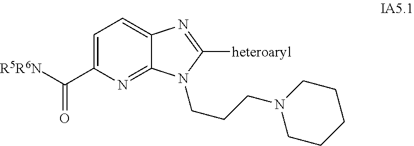

wherein R⁵, R⁶, and heteroaryl are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula IA6, there is provided a compound of Formula IA6.1

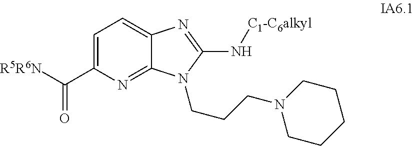

wherein R⁵, R⁶, and heteroaryl are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula IA7, there is provided a compound of Formula IA7.1

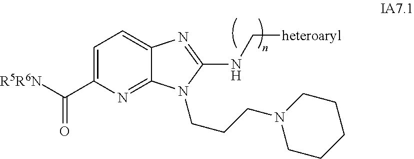

wherein R⁵, R⁶, and heteroaryl are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula IA8, there is provided a compound of Formula IA8.1

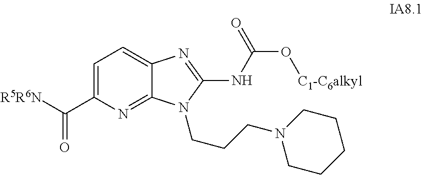

wherein R⁵, R⁶, and heteroaryl are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula IA9, there is provided a compound of Formula IA9.1

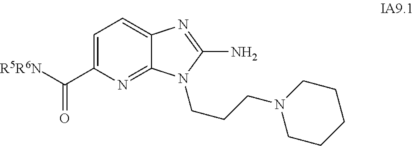

wherein R⁵, R⁶, and heteroaryl are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula IA10, there is provided a compound of Formula IA10.1

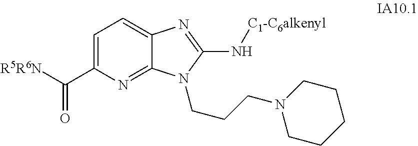

wherein R⁵, R⁶, and heteroaryl are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula IA11, there is provided a compound of Formula IA11.1

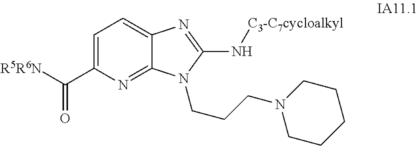

wherein R⁵, R⁶, and heteroaryl are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula IA12, there is provided a compound of Formula IA12.1

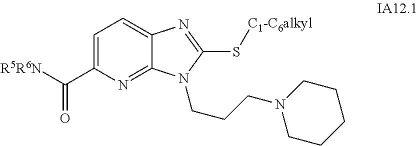

wherein R⁵, R⁶, and heteroaryl are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula IA13, there is provided a compound of Formula IA13.1

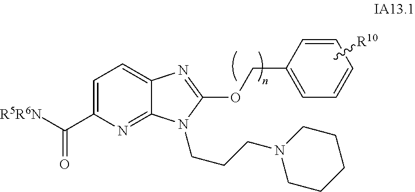

wherein R⁵, R⁶, and heteroaryl are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula IA16, there is provided a compound of Formula IA16.1:

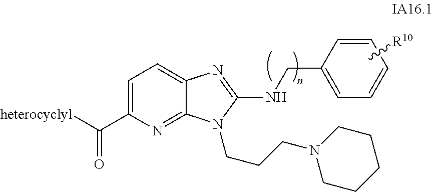

wherein n is an integer from 0 to 1; R⁵, R⁶, R¹⁰ and heterocyclyl are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula IA30, there is provided a compound of Formula IA30.1

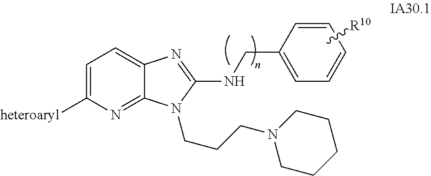

wherein n is an integer from 0 to 1; R¹⁰ and heteroaryl are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula IA2, there is provided a compound of Formula IA2.2

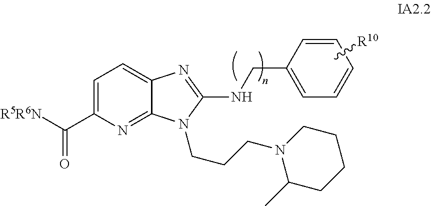

wherein n is an integer from 0 to 1; R⁵, R⁶, and R¹⁰ are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula IA2, there is provided a compound of Formula IA2.3

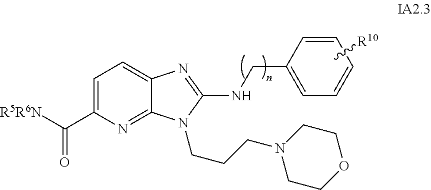

wherein n is an integer from 0 to 1; R⁵, R⁶, and R¹⁰ are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula IA2, there is provided a compound of Formula IA2.4

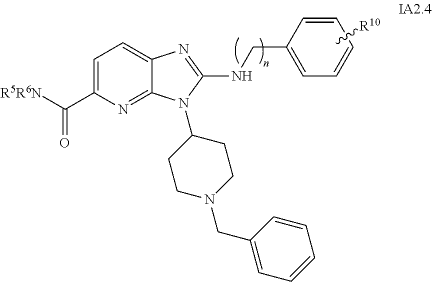

wherein n is an integer from 0 to 1; R⁵, R⁶, and R¹⁰ are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula IA2, there is provided a compound of Formula IA2.5

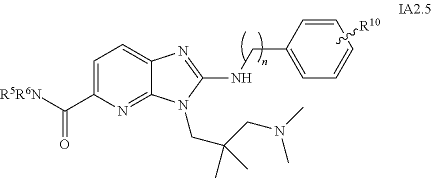

wherein n is an integer from 0 to 1; R⁵, R⁶, and R¹⁰ are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula IA3, there is provided a compound of Formula IA3.2

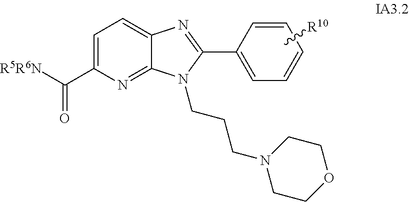

wherein n is an integer from 0 to 1; R⁵, R⁶, and R¹⁰ are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula IA7, there is provided a compound of Formula IA7.2

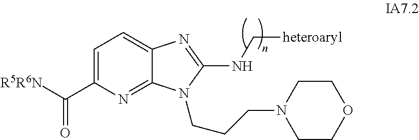

wherein n is an integer from 0 to 1; R⁵, R⁶, and R¹⁰ are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula IA14, there is provided a compound of Formula IA14.2

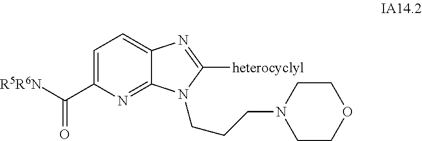

wherein n is an integer from 0 to 1; R⁵, R⁶, and R¹⁰ are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula IB3, there is provided a compound of Formula IB2.1

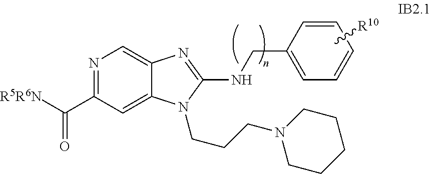

wherein n is an integer from 0 to 1; R⁵, R⁶, and R¹⁰ are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula IB3, there is provided a compound of Formula IB3.1

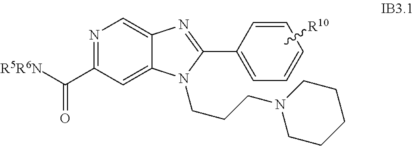

wherein n is an integer from 0 to 1; R⁵, R⁶, and R¹⁰ are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula IB5, there is provided a compound of Formula IB5.1

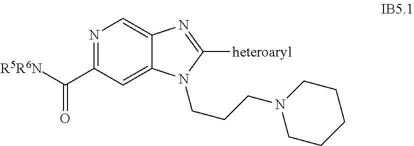

wherein n is an integer from 0 to 1; R⁵, R⁶, and R¹⁰ are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula IB13, there is provided a compound of Formula IB13.1

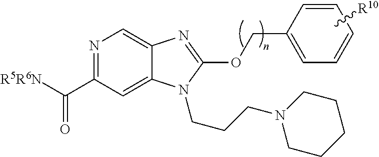

wherein n is an integer from 0 to 1; R⁵, R⁶, and R¹⁰ are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula IC2, there is provided a compound of Formula IC2.1

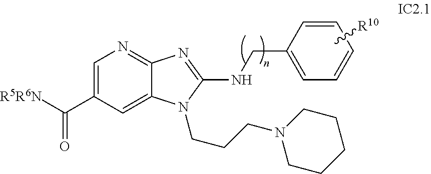

wherein n is an integer from 0 to 1; R⁵, R⁶, and R¹⁰ are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula ID3, there is provided a compound of Formula ID3.1

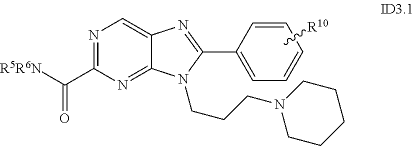

wherein n is an integer from 0 to 1; R⁵, R⁶, and R¹⁰ are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula IE2, there is provided a compound of Formula IE2.1

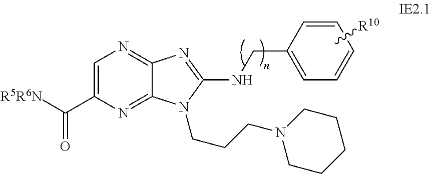

wherein n is an integer from 0 to 1; R⁵, R⁶, and R¹⁰ are as defined hereinabove and hereinbelow; or a prodrug; or a pharmaceutically acceptable salt to allow the drug to penetrate the cell membrane; or the compound is labeled with a detectable label or an affinity tag thereof.

In one subset of compounds of Formula IF3, there is provided a compound of Formula IF3.1

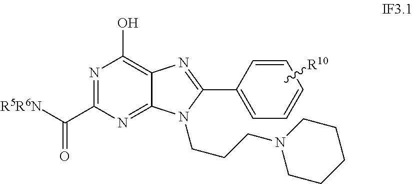

wherein n is an integer from 0 to 1; R⁵, R⁶, and R¹⁰ are as defined hereinabove and hereinbelow; or a prodrug; or a pharmaceutically acceptable salt to allow the drug to penetrate the cell membrane; or the compound is labeled with a detectable label or an affinity tag thereof.

### X:

In one subset of compounds, X is N, and X¹ and X² are both CH.

Any and each individual definition of X as set out herein may be combined with any and each individual definition of X¹, X², R¹, R² and R³ as set out herein.

### X¹:

In one alternative subset of compounds, X¹ is N, and X and X² are both CH.

Any and each individual definition of X¹ as set out herein may be combined with any and each individual definition of X, X², R¹, R² and R³ as set out herein.

In one alternative subset of compounds, X¹ and X are N, and X² is CH.

Any and each individual definition of X¹ as set out herein may be combined with any and each individual definition of X, X², R¹, R² and R³ as set out herein.

In one alternative subset of compounds, X¹ and X are N, and X² is C—OH.

Any and each individual definition of X¹ as set out herein may be combined with any and each individual definition of X, X², R¹, R² and R³ as set out herein.

### X²:

In another alternative subset of compounds, X² is N, and X and X¹ are both CH.

Any and each individual definition of X² as set out herein may be combined with any and each individual definition of X, X¹, R¹, R² and R³ as set out herein.

In one alternative subset of compounds, X and X² are N and X¹ is CH

Any and each individual definition of X¹ as set out herein may be combined with any and each individual definition of X, X², R¹, R² and R³ as set out herein.

### R¹:

In one subset of compounds, R¹ is
1) C₁-C₆ alkyl,
2) aryl,
3) heteroaryl,
4) NHR⁴,
5) C₁-C₆ alkyl-aryl,
6) C₁-C₆ alkyl-heteroaryl,
7) C(O) aryl,
8) C(O) heteroaryl,
9) OR⁴,
10) SR⁴, or
11) heterocyclyl,
   wherein the aryl, the heteroaryl and the heterocyclyl are optionally substituted with one or more R¹⁰ substituents.

In one subset of compounds, R¹ is C₁-C₆ alkyl. In one example, R¹ is CH₃.

In an alternative subset of compounds, R¹ is aryl optionally substituted with one R¹⁰ substituent.

In one example, R¹ is phenyl optionally substituted with one R¹⁰ substituent.

In an alternative subset of compounds, R¹ is heteroaryl optionally substituted with one R¹⁰ substituent. In one example, R¹ is benzothiophene optionally substituted with one R¹⁰ substituent.

In an alternative subset of compounds, R¹ is NHR⁴. In one example, R¹ is NHC(O)-phenyl, in which phenyl is optionally substituted with one R¹⁰ substituent.

In another example, R¹ is NHC(O)—OC₁-C₆alkyl. In one specific example, R¹ is NHCOOCH₃.

In another example, R¹ is NHC₁-C₆alkyl. In one specific example R¹ is NHCH(CH₃)₂.

In another example, R¹ is NH₂.

In yet another example, R¹ is NHC₂-C₆alkenyl. In one specific example R¹ is NHmethallyl.

In still another example, R¹ is NHC₃-C₇cycloalkyl. In one specific example R¹ is NHcyclohexyl.

In one example, R¹ is SC₁-C₆alkyl. In one specific example R¹ is SCH₂CH₃.

In another example, R¹ is OR⁴.

Any and each individual definition of R¹ as set out herein may be combined with any and each individual definition of X, X¹, X², R² and R³ as set out herein.

### R²:

In one subset of compounds, R² is
1) C₁-C₆ alkyl-heterocyclyl,
2) C₁-C₆ alkyl-N(C₁-C₆ alkyl)₂,
3) C₁-C₆ alkyl-NHC₁-C₆ alkyl,
4) C₁-C₆ alkyl-NH₂, or
5) heterocyclyl,
   wherein the heterocyclyl is optionally substituted with one or more C₁-C₆ alkyl substituents or C₁-C₆ alkyl-aryl.

In one example, R² is C₁-C₆ alkyl-heterocyclyl.

In one example, R² is C₁-C₆ alkyl-N(C₁-C₆ alkyl)₂, In an alternative example, R² is heterocyclyl.

Any and each individual definition of R² as set out herein may be combined with any and each individual definition of X, X¹, X², R¹ and R³ as set out herein.

### R³:

In one subset of compounds, R³ is
1) C(O)NR⁵R⁶,
2) CH₂NR⁵R⁶,
3) C(O)-heterocyclyl,
4) heteroaryl,
5) aryl,
6) C(NH)NR⁵R⁶, or
7) CH(CF₃)NR⁵R⁶
   wherein the heteroaryl or the heterocyclyl are optionally substituted with one or more R¹⁰ substituents;

In one example, R³ is C(O)NR⁵R⁶.

In an alternative example, R³ is C(O)-heterocyclyl in which the heterocyclyl is optionally substituted with one or more R¹⁰ substituents.

In an alternative example of compounds, R³ is heteroaryl in which the heteroaryl is optionally substituted with one or more R¹⁰ substituents.

Any and each individual definition of R³ as set out herein may be combined with any and each individual definition of X, X¹, X², R¹ and R² as set out herein.

### R⁴:

In one subset of compounds, R⁴ is
1) H,
2) aryl,
3) heteroaryl,
4) C₁-C₆ alkyl,
5) C₁-C₆ alkyl-aryl,
6) C₁-C₆ alkyl-heteroaryl,
7) C₁-C₆alkyl-NHC(O) C₁-C₆ alkyl,
8) C₁-C₆ alkyl-O—C₁-C₆ alkyl,
9) C₁-C₆ alkyl-heterocyclyl,
10) C₂-C₆ alkenyl,
11) C₃-C₇ cycloalkyl,
12) C(O)-aryl, or
13) C(O)O C₁-C₆ alkyl
   wherein the aryl, the heteroaryl or the heterocyclyl is optionally substituted with one or more R¹⁰ substituents.

In one example, R⁴ is phenyl optionally substituted with one R¹⁰ substituent.

In an alternative example, R⁴ is C₁-C₆ alkyl-phenyl, wherein the phenyl is optionally substituted with one R¹⁰ substituent.

Any and each individual definition of R⁴ as set out herein may be combined with any and each individual definition of X, X¹, X², R¹, R² and R³ as set out herein.

R⁵ and R⁶:

In one subset of compounds, R⁵ and R⁶ are each independently selected from
1) C₁-C₆ alkyl,
2) C₁-C₆ alkyl-C₃-C₇ cycloalkyl,
3) C₁-C₆ alkyl-O—C₁-C₆ alkyl, or
4) C₁-C₆ alkyl-CN.

In one example, both R⁵ and R⁶ are C₁-C₆ alkyl.

In an alternative example, both R⁵ and R⁶ are C₁-C₆ alkyl-C₃-C₇ cycloalkyl.

In an alternative example, R⁵ is C₁-C₆ alkyl and R⁶ is C₁-C₆ alkyl-O—C₁-C₆ alkyl.

In an alternative example, both R⁵ and R⁶ are C₁-C₆ alkyl-O—C₁-C₆ alkyl.

In an alternative example, R⁵ is C₁-C₆ alkyl and R⁶ is C₁-C₆ alkyl-CN

Any and each individual definition of R⁵ and R⁶ as set out herein may be combined with any and each individual definition of X, X¹, X², R¹, R² and R³ as set out herein.

### R¹⁰:

In one subset of compounds, R¹⁰ is
1) CN,
2) NO₂,
3) halogen,
4) haloalkyl,
5) C₁-C₆ alkyl,
6) OH,
7) OC₁-C₆ alkyl,
8) O-aryl,
9) C(O)OH,
10) C(O)OC₁-C₆ alkyl,
11) C(O) C₁-C₆ alkyl,
12) C(O) aryl,
13) C(O) heterocyclyl,
14) C(O)NH₂,
15) C(O)NH C₁-C₆ alkyl,
16) C(O)N(C₁-C₆ alkyl)₂,
17) NH₂,
18) NHC(O) C₁-C₆ alkyl,
19) C₁-C₆ alkyl heterocyclyl,
20) C₁-C₆ alkyl (OH),
21) aryl,
22) heteroaryl, or
23) heterocyclyl,

wherein the heterocyclyl is optionally substituted with one or more C₁-C₆ alkyl substituents.

In one example, R¹⁰ is CN.

In one example, R¹⁰ is F, Cl, or Br. In one specific example R¹⁰ is Br.

In another example, R¹⁰ is C₁-C₆ alkyl. In one specific example, R¹⁰ is CH₃.

In another example, R¹⁰ is OC₁-C₆ alkyl. In one specific example, R¹⁰ is OCH₃.

In another example, R¹⁰ is C(O)OC₁-C₆ alkyl.

In another example, R¹⁰ is C(O)C₁-C₆ alkyl. In one specific example R¹⁰ is C(O)OCH₃.

In another example, R¹⁰ is C(O)OH.

In another example, R¹⁰ is C(O)N(C₁-C₆ alkyl)₂.

In another example, R¹⁰ is C(O)heterocyclyl.

In another example, R¹⁰ is 3,4-methylenedioxy ketal.

In another example, R¹⁰ is C(OH)C₁-C₆ alkyl

Any and each individual definition of R¹⁰ as set out herein may be combined with any and each individual definition of X, X¹, X², R¹, R² and R³ as set out herein.

### DEFINITIONS

Unless otherwise specified, the following definitions apply:

The singular forms “a”, “an” and “the” include corresponding plural references unless the context clearly dictates otherwise.

As used herein, the term “comprising” is intended to mean that the list of elements following the word “comprising” are required or mandatory but that other elements are optional and may or may not be present.

As used herein, the term “consisting of” is intended to mean including and limited to whatever follows the phrase “consisting of”. Thus the phrase “consisting of” indicates that the listed elements are required or mandatory and that no other elements may be present.

As used herein, the term “alkyl” is intended to include both branched and straight chain saturated aliphatic hydrocarbon groups having the specified number of carbon atoms, for example, C₁-C₆ as in C₁-C₆ alkyl is defined as including groups having 1,2,3,4,5 or 6 carbons in a linear or branched arrangement. Examples of C₁-C₆ alkyl as defined above include, but are not limited to, methyl, ethyl, n-propyl, i-propyl, n-butyl, t-butyl, i-butyl, pentyl, and hexyl.

As used herein, the term “cycloalkyl” is intended to mean a monocyclic saturated aliphatic hydrocarbon group having the specified number of carbon atoms therein, for example, C₃-C₇ as in C₃-C₇ cycloalkyl is defined as including groups having 3,4,5,6, or 7 carbons in a monocyclic arrangement. Examples of C₃-C₇ cycloalkyl as defined above include, but are not limited to, cyclopropyl, cyclobutyl, cyclopentyl, cyclohexyl and cycloheptyl.

As used herein, the term “alkenyl” is intended to include both branched and straight chain aliphatic hydrocarbon groups containing at least one unsaturated carbon-carbon double bond having the specified number of carbon atoms therein, for example, C₁-C₆ as in C₁-C₆ alkenyl is defined as including groups having 1,2,3,4,5 or 6 carbons and at least one unsaturated C—C bond in a linear or branched arrangement.

As used herein, the term “halo” or “halogen” is intended to mean fluorine, chlorine, bromine and iodine.

As used herein, the term “haloalkyl” is intended to mean an alkyl as defined above, in which each hydrogen atom may be successively replaced by a halogen atom. Examples of haloalkyls include, but are not limited to, CH₂F, CHF₂ and CF₃.

As used herein, the term “aryl”, either alone or in combination with another radical, means a carbocyclic aromatic monocyclic group containing 6 carbon atoms which may be further fused to a second 5- or 6-membered carbocyclic group which may be aromatic, saturated or unsaturated. Aryl includes, but is not limited to, phenyl, indanyl, 1-naphthyl, 2-naphthyl and tetrahydronaphthyl. The aryls may be connected to another group either at a suitable position on the cycloalkyl ring or the aromatic ring.

As used herein, the term “heteroaryl” is intended to mean a monocyclic or bicyclic ring system of up to ten atoms, wherein at least one ring is aromatic, and contains from 1 to 4 hetero atoms selected from the group consisting of O, N, and S. The heteroaryl substituent may be attached either via a ring carbon atom or one of the heteroatoms. Examples of heteroaryl groups include, but are not limited to thienyl, benzimidazolyl, benzo[b]thienyl, furyl, benzofuranyl, pyranyl, isobenzofuranyl, chromenyl, xanthenyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, napthyridinyl, quinoxalinyl, quinazolinyl, cinnolinyl, pteridinyl, isothiazolyl, isochromanyl, chromanyl, isoxazolyl, furazanyl, indolinyl, isoindolinyl, thiazolo[4,5-b]-pyridine, and fluoroscein derivatives.

As used herein, the term “heterocycle”, “heterocyclic” or “heterocyclyr” is intended to mean a 5, 6, or 7 membered non-aromatic ring system containing from 1 to 4 heteroatoms selected from the group consisting of O, N and S. Examples of heterocycles include, but are not limited to pyrrolidinyl, tetrahydrofuranyl, piperidyl, 3,5-dimethylpiperidyl, pyrrolinyl, piperazinyl, imidazolidinyl, morpholinyl, imidazolinyl, pyrazolidinyl, pyrazolinyl.

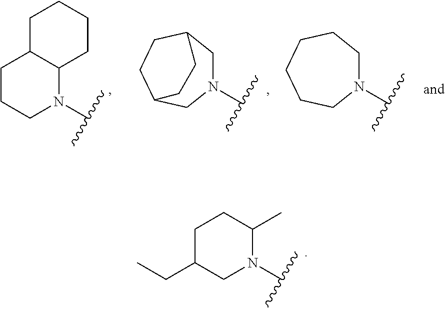

As used herein, the term “modulating the activity of melanocortin-4 receptor” is intended to mean a compound which, upon binding to the receptor, elicits a physiological response such as, but not limited to, regulation of food intake and energy expenditure. The compounds can be agonists, antagonists, partial agonists and inverse agonists. Generally, all diseases and disorders where the regulation of MC4R is involved can be treated with the compounds described herein.

As used herein, the term “detectable label” is intended to mean a group that may be linked to a compound of Formula I to produce a probe or to a melanocortin-4 receptor, such that when the probe is associated with the melanocortin-4 receptor, the label allows either direct or indirect recognition of the probe so that it may be detected, measured and quantified.

As used herein, the term “affinity tag” is intended to mean a ligand or group, which is linked to either a compound of Formula I or to a melanocortin-4 receptor to allow another compound to be extracted from a solution to which the ligand or group is attached.

As used herein, the term “probe” is intended to mean a compound of Formula I, which is labeled with either a detectable label or an affinity tag, and which is capable of binding, either covalently or non-covalently, to a melanocortin-4 receptor. When, for example, the probe is non-covalently bound, it may be displaced by a test compound. When, for example, the probe is bound covalently, it may be used to form cross-linked adducts, which may be quantified and inhibited by a test compound.

As used herein, the term “optionally substituted with one or more substituents” or its equivalent term “optionally substituted with at least one substituent” is intended to mean that the subsequently described event of circumstances may or may not occur, and that the description includes instances where the event or circumstance occurs and instances in which it does not. The definition is intended to mean from zero to five substituents.

If the substituents themselves are incompatible with the synthetic methods described herein, the substituent may be protected with a suitable protecting group (PG) that is stable to the reaction conditions used in these methods. The protecting group may be removed at a suitable point in the reaction sequence of the method to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (4th ed.), John Wiley & Sons, NY (2007), which is incorporated herein by reference in its entirety. Examples of protecting groups used throughout include, but are not limited to Fmoc, Bn, Boc, CBz and COCF₃. In some instances, a substituent may be specifically selected to be reactive under the reaction conditions used in the methods described herein. Under these circumstances, the reaction conditions convert the selected substituent into another substituent that is either useful in an intermediate compound in the methods described herein or is a desired substituent in a target compound.

As used herein, the term “prodrug” is intended to mean a compound that may be converted under physiological conditions or by solvolysis to a biologically active compound of Formula I. Thus, the term “prodrug” refers to a precursor of a compound of Formula I that is pharmaceutically acceptable. A prodrug may be inactive or display limited activity when administered to a subject in need thereof, but is converted in vivo to an active compound of Formula I. Typically, prodrugs are transformed in vivo to yield the compound of Formula I, for example, by hydrolysis in blood or other organs by enzymatic processing. The prodrug compound often offers advantages of solubility, tissue compatibility or delayed release in the subject (see, Bundgard, Design of Prodrugs (1985), pp. 7-9, 21-24 (Elsevier, Amsterdam). The definition of prodrug includes any covalently bonded carriers which release the active compound described herein in vivo when such prodrug is administered to a subject. Prodrugs of a compound of Formula I may be prepared by modifying functional groups present in the compound of Formula I in such a way that the modifications are cleaved, either in routine manipulation or in vivo, to a parent compound of Formula I.

As used herein, the term “pharmaceutically acceptable salt” is intended to mean both acid and base addition salts.

As used herein, the term “pharmaceutically acceptable acid addition salt” is intended to mean those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

As used herein, the term “pharmaceutically acceptable base addition salt” is intended to mean those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like.

The compounds of Formula I, or their pharmaceutically acceptable salts may contain one or more asymmetric centers, chiral axes and chiral planes and may thus give rise to enantiomers, diastereomers, and other stereoisomeric forms and may be defined in terms of absolute stereochemistry, such as (R)- or (S)- or, as (D)- or (L)-for amino acids. The present is intended to include all such possible isomers, as well as, their racemic and optically pure forms. Optically active (+) and (−), (R)- and (S)-, or (D)- and (L)-isomers may be prepared using chiral synthons or chiral reagents, or resolved using conventional techniques, such as reverse phase HPLC.

The racemic mixtures may be prepared and thereafter separated into individual optical isomers or these optical isomers may be prepared by chiral synthesis. The enantiomers may be resolved by methods known to those skilled in the art, for example by formation of diastereoisomeric salts which may then be separated by crystallization, gas-liquid or liquid chromatography, selective reaction of one enantiomer with an enantiomer specific reagent. It will also be appreciated by those skilled in the art that where the desired enantiomer is converted into another chemical entity by a separation technique, an additional step is then required to form the desired enantiomeric form. Alternatively specific enantiomers may be synthesized by asymmetric synthesis using optically active reagents, substrates, catalysts, or solvents or by converting one enantiomer to another by asymmetric transformation.

Certain compounds of Formula I may exist as a mix of epimers. Epimers means diastereoisomers that have the opposite configuration at only one of two or more stereogenic centres present in the respective compound.

Certain compounds of Formula I may exist in Zwitterionic form and the present includes Zwitterionic forms of these compounds and mixtures thereof.

In addition, the compounds of Formula I also may exist in hydrated and anhydrous forms. Hydrates of the compound of any of the formulas described herein are included. In a further embodiment, the compound according to any of the formulas described herein is a monohydrate. In one embodiment, the compounds described herein comprise about 10% or less, about 9% or less, about 8% or less, about 7% or less, about 6% or less, about 5% or less, about 4% or less, about 3% or less, about 2% or less, about 1% or less, about 0.5% or less, about 0.1% or less by weight of water. In another embodiment, the compounds described herein comprise, about 0.1% or more, about 0.5% or more, about 1% or more, about 2% or more, about 3% or more, about 4% or more, about 5% or more, or about 6% or more by weight of water.

As used herein, the term “melanocortin-4 receptor” is intended to mean a G-protein coupled receptor encoded by the MC4R gene and that binds melanocortins, Agouti and Agouti-related protein.

As used herein, the term “therapeutically effective amount” is intended to mean an amount of a compound of Formula I which, when administered to a subject is sufficient to effect treatment for a disease-state mediated by a melanocortin-4 receptor. The amount of the compound of Formula I will vary depending on the compound, the condition and its severity, and the age of the subject to be treated, but can be determined routinely by one of ordinary skill in the art having regard to his own knowledge and to this disclosure.

As used herein, the term “treating” or “treatment” is intended to mean treatment of a disease-state mediated by a melanocortin-4 receptor, as disclosed herein, in a subject, and includes: (i) preventing a disease or condition mediated by a melanocortin-4 receptor from occurring in a subject, in particular, when such mammal is predisposed to the disease or condition but has not yet been diagnosed as having it; (ii) inhibiting a disease or condition mediated by a melanocortin-4 receptor, i.e., arresting its development; or (iii) relieving a disease/disorder or condition mediated by a melanocortin-4 receptor, i.e., causing regression of the condition.

As used herein, the term “treating obesity” is intended to mean the administration of a pharmaceutical composition described herein to a subject, preferably a human, which is afflicted with obesity to cause an alleviation of the obesity.

As used herein, the term “IC₅₀” is intended to mean an amount, concentration or dosage of a particular compound of Formula I that achieves a 50% inhibition of a maximal agonist response.

As used herein, the term “EC₅₀” is intended to mean an amount, concentration or dosage of a particular compound of Formula I that achieves a 50% of its maximal effect.

As used herein, the term “modulate” or “modulating” is intended to mean the treatment, prevention, suppression, enhancement or induction of a function or condition using the compounds as described herein. For example, the compounds as described herein can modulate melanocortin-4 receptor function in a subject, thereby altering or regulating the activity of melanocortin-4 receptor (MC4R) and consequently modifying physiological responses associated with MC4R such as, but not limited to, food intake and energy expenditure. In this context, compounds as described herein can elicit an effect useful for the treatment of cachexia, eating disorders, diabetes, metabolic diseases, erectile and/or sexual dysfunction.

### 3. Utilities

The compounds as described herein are useful as melanocortin-4 receptor modulating compounds and as such the compounds, compositions and methods described herein include application to the cells or subjects afflicted with or having a predisposition towards developing a particular disease state, which is mediated by a melanocortin-4 receptor. Thus, the compounds, compositions and methods are used to treat diseases/disorders, which include, but are not limited to, obesity, cachexia, eating disorders, diabetes, metabolic diseases, erectile and sexual dysfunction.

The treatment involves administration to a subject in need thereof a compound of Formula I or a pharmaceutically acceptable salt thereof, or a pharmaceutical composition comprising a pharmaceutical carrier and a therapeutically effective amount of a compound of Formula I, or a pharmaceutically acceptable salt thereof. In particular, the compounds, compositions and methods described herein are useful in the treatment of obesity . . . .

The compounds described herein, or their pharmaceutically acceptable salts or their prodrugs, may be administered in pure form or in an appropriate pharmaceutical composition, and can be carried out via any of the accepted modes of Galenic pharmaceutical practice.

The pharmaceutical compositions described herein can be prepared by mixing a compound of described herein with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral (subcutaneous injections, intravenous, intramuscular, intrastemal injection or infusion techniques), sublingual, ocular, rectal, vaginal, and intranasal. Pharmaceutical compositions described herein are formulated so as to allow the active ingredients contained therein to be bioavailable upon administration of the composition to a subject. Compositions that will be administered to a subject or patient take the form of one or more dosage units, where for example, a tablet may be a single dosage unit, and a container of a compound described herein in aerosol form may hold a plurality of dosage units. Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound described herein, or a pharmaceutically acceptable salt thereof, for treatment of a disease-state as described above.

A pharmaceutical composition described herein may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example inhalatory administration.

For oral administration, the pharmaceutical composition is typically in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, e.g., a gelatin capsule, it may contain, in addition to materials of the above type, a liquid carrier such as polyethylene glycol or oil such as soybean or vegetable oil.

The pharmaceutical composition may be in the form of a liquid, e.g., an elixir, syrup, solution, emulsion or suspension. The liquid may be for oral administration or for delivery by injection, as two examples. When used for oral administration, a typical composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions described herein, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; encapsulating agents such as cyclodextrins or functionalized cyclodextrins, including, but not limited to, α, β or δ-hydroxypropylcyclodextins or Captisol; antioxidants such as ascorbic acid or sodium bisulfite; chelating agents such as ethylenediamine tetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is typically sterile.

A liquid pharmaceutical composition may be used for either parenteral or oral administration should contain an amount of a compound described herein such that a suitable dosage will be obtained. Typically, this amount is at least 0.01% of a compound described herein in the composition. When intended for oral administration, this amount may be varied to be between 0.1 and about 70% of the weight of the composition. For parenteral usage, compositions and preparations described herein are prepared so that a parenteral dosage unit contains between 0.01 to 10% by weight of the compound described herein. Pharmaceutical compositions may be further diluted at the time of administration; for example a parenteral formulation may be further diluted with a sterile, isotonic solution for injection such as 0.9% saline, 5 wt % dextrose (D5W), Ringer's solution, or others.

The pharmaceutical composition described herein may be used for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the compound described herein from about 0.1 to about 10% w/v (weight per unit volume).

The pharmaceutical composition described herein may be used for rectal administration of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition described herein may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition described herein in solid or liquid form may include an agent that binds to the compound described herein and thereby assists in the delivery of the compound. Suitable agents that may act in this capacity include, but are not limited to, a monoclonal or polyclonal antibody, a protein or a liposome.

The pharmaceutical composition described herein may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds described herein may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

The pharmaceutical compositions described herein may be prepared by methodology well known in the pharmaceutical art. For example, a pharmaceutical composition intended to be administered by injection can be prepared by mixing a compound described herein with sterile, distilled water so as to form a solution. A surfactant may be added to facilitate the formation of a homogeneous solution or suspension. Surfactants are compounds that non-covalently interact with the compound described herein so as to facilitate dissolution or homogeneous suspension of the compound in the aqueous delivery system.

The compounds described herein, or their pharmaceutically acceptable salts, may be administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose may be from about 0.1 mg to about 40 mg/kg of body weight per day or twice per day of a compound described herein, or a pharmaceutically acceptable salt thereof.

### 4. Screening Assays

The compounds described herein may also be used in a method to screen for other compounds that bind to a melanocortin-4 receptor. Generally speaking, to use the compounds described herein in a method of identifying compounds that bind to a melanocortin-4 receptor, the receptor is bound to a support, and a compound described herein is added to the assay. Alternatively, the compound may be bound to the support and the receptor is added.

There are a number of ways in which to determine the binding of a compound described herein to the melanocortin-4 receptor. In one way, the compound, for example, may be fluorescently or radioactively labeled and binding determined directly. For example, this may be done by attaching the receptor to a solid support, adding a detectably labeled compound, washing off excess reagent, and determining whether the amount of the detectable label is that present on the solid support. Numerous blocking and washing steps may be used, which are known to those skilled in the art.

In some cases, only one of the components is labeled. For example, specific residues in the receptor may be labeled. Alternatively, more than one component may be labeled with different labels; for example, using ¹²⁵I for the receptor, and a fluorescent label for the probe.

The compounds described herein may also be used as competitors to screen for additional drug candidates or test compounds. As used herein, the terms “drug candidate” or “test compounds” are used interchangeably and describe any molecule, for example, protein, oligopeptide, small organic molecule, polysaccharide, polynucleotide, and the like, to be tested for bioactivity. The compounds may be capable of directly or indirectly altering the melanortin-4 receptor biological activity.

Drug candidates can include various chemical classes, although typically they are small organic molecules having a molecular weight of more than 100 and less than about 2,500 Daltons. Candidate agents typically include functional groups necessary for structural interaction with proteins, for example, hydrogen bonding and lipophilic binding, and typically include at least an amine, carbonyl, hydroxyl, ether, or carboxyl group. The drug candidates often include cyclical carbon or heterocyclic structures and/or aromatic or polyaromatic structures substituted with one or more functional groups.

Drug candidates can be obtained from any number of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means.

Competitive screening assays may be done by combining an melanocortin-4 receptor and a probe to form a probe:receptor complex in a first sample followed by adding a test compound from a second sample. The binding of the test is determined, and a change or difference in binding between the two samples indicates the presence of a test compound capable of binding to the receptor and potentially modulating the receptor's activity.

In one case, the binding of the test compound is determined through the use of competitive binding assays. In this example, the probe is labeled with a fluorescent label. Under certain circumstances, there may be competitive binding between the test compound and the probe. Test compounds which display the probe, resulting in a change in fluorescence as compared to control, are considered to bind to the melanocortin-4 receptor.

In one case, the test compound may be labeled. Either the test compound, or a compound described herein, or both, is added first to the melanocortin-4 receptor for a time sufficient to allow binding to form a complex.

Formation of the probe:receptor complex typically require Incubations of between 4° C. and 40° C. for between 10 minutes to about 1 hour to allow for high-throughput screening. Any excess of reagents are generally removed or washed away. The test compound is then added, and the presence or absence of the labeled component is followed, to indicate binding to the receptor.

In one case, the probe is added first, followed by the test compound. Displacement of the probe is an indication the test compound is binding to the melanocortin-4 receptor and thus is capable of binding to, and potentially modulating, the activity of the receptor. Either component can be labeled. For example, the presence of probe in the wash solution indicates displacement by the test compound. Alternatively, if the test compound is labeled, the presence of the probe on the support indicates displacement.

In one case, the test compound may be added first, with incubation and washing, followed by the probe. The absence of binding by the probe may indicate the test compound is bound to the melanocortin-4 receptor with a higher affinity. Thus, if the probe is detected on the support, coupled with a lack of test compound binding, may indicate the test compound is capable of binding to the receptor.

Modulation is tested by screening for a test compound's ability to modulate the activity of melanocortin-4 receptor and includes combining a test compound with the receptor, as described above, and determining an alteration in the biological activity of the receptor. Therefore in this case, the test compound should both bind to the receptor (although this may not be necessary), and alter its biological activity as defined herein.

Positive controls and negative controls may be used in the assays. All control and test samples are performed multiple times to obtain statistically significant results. Following incubation, all samples are washed free of non-specifically bound material and the amount of bound probe determined. For example, where a radiolabel is employed, the samples may be counted in a scintillation counter to determine the amount of bound compound.

Typically, the signals that are detected in the assay may include fluorescence, resonance energy transfer, time resolved fluorescence, radioactivity, fluorescence polarization, plasma resonance, or chemiluminescence and the like, depending on the nature of the label. Detectable labels useful in performing screening assays described herein include a fluorescent label such as Fluorescein, Oregon green, dansyl, rhodamine, tetramethyl rhodamine, Texas Red, Eu³⁺; a chemiluminescent label such as luciferase; colorimetric labels; enzymatic markers; or radioisotopes such as tritium, I¹²⁵ and the like

Affinity tags, which may be useful in performing the screening assay described herein include be biotin, polyhistidine and the like.

### Synthesis and Methodology

General methods for the synthesis of the compounds described herein are shown below and are disclosed merely for the purpose of illustration and are not meant to be interpreted as limiting the processes to make the compounds by any other methods. Those skilled in the art will appreciate that a number of methods are available for the preparation of compounds described herein.

Schemes 1 to 12 illustrate general synthetic procedures for the preparation of compounds described herein.

Scheme 1 describes a general synthetic approach to the compounds described herein. Compounds 1-ix are prepared by the following sequence. Dihalogenated compounds such as 1-i are treated with an amine 1-ii to provide the monohalogenated compounds 1-iii. Reduction to the intermediates 1-iv is carried out using a number of methods known by those skilled in the art. Intermediates 1-iv are treated with electrophilic compounds 1-v in the presence of a coupling, dehydrating or oxidizing agent to yield the imidazopyridine intermediates 1-vi. The compounds described herein, e.g. 1-ix, are finally obtained by treatment with nucleophiles 1-vii under various carbonylation methods known by those skilled in the art. Alternatively, alkyl esters 1-ix (L=CO; A=OR) can be converted directly to the amides 1-ix (L=CO; A=NR⁵R⁶) by treatment with an amine and trimethylaluminium. Nucleophiles 1-vii yielding to compounds described in Table are all available form commercial sources except for Example 37. In the latter case, the required bis(2-cyclopropylethyl)amine was prepared according to the sequence described in patent application WO08116665 page 84.

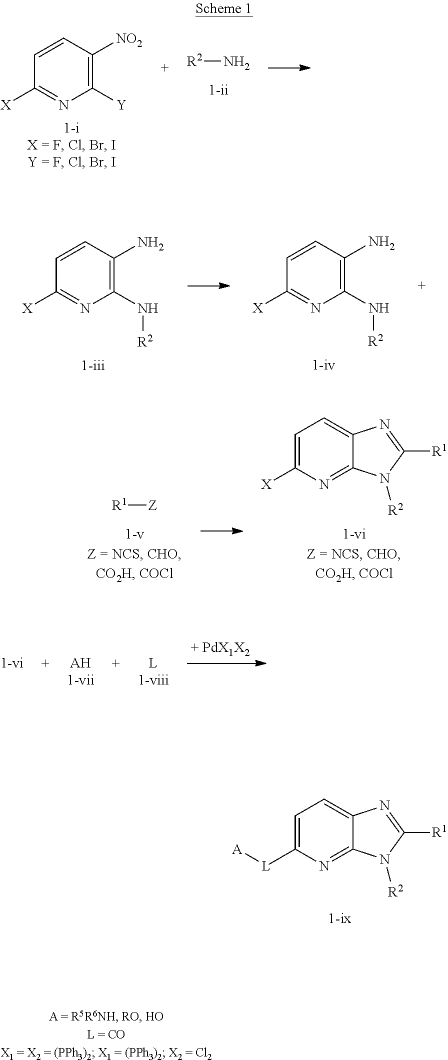

Scheme 2 describes another general synthetic approach to the compounds described herein. Compounds 1-ix are prepared by the following sequence. Intermediates 1-iii are treated with nucleophiles 1-vii under various carbonylation methods known by those skilled in the art to afford intermediates 2-i. Reduction to the intermediate 2-ii is carried out using a number of methods known by those skilled in the art. The compounds described herein 1-ix are finally obtained by treatment with electrophilic compounds 2-iii in the presence of a coupling, dehydrating or oxidizing agent.

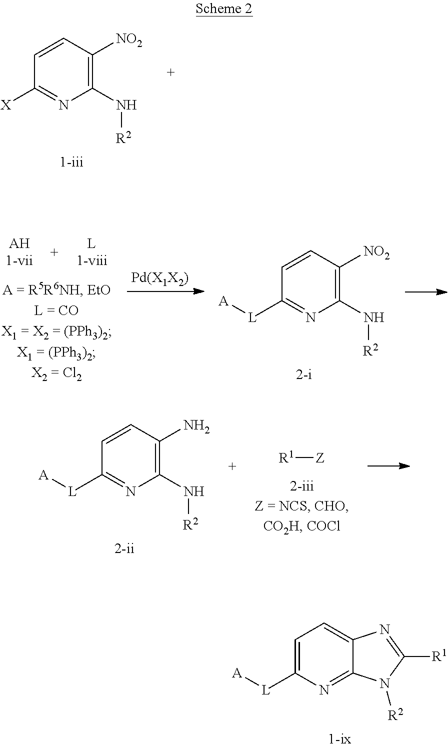

Alternatively, scheme 3 describes a modification of the general synthetic approach to the compounds described herein. Compounds 3-viii are prepared by the following sequence. Intermediates 1-iii are treated with nucleophiles 3-i under various carbonylation methods known by those skilled in the art to afford intermediates 3-ii. Hydrolysis or saponification of ester intermediates 3-ii to compounds 3iii allows amide coupling with nucleophiles 3-iv affording intermediates 3-v. Reduction of the nitro group to the intermediates 3-vi is carried out using a number of methods known by those skilled in the art. The compounds described herein 3-viii are finally obtained by treatment with electrophilic compounds 3-vii in the presence of a coupling, dehydrating or oxidizing agent.

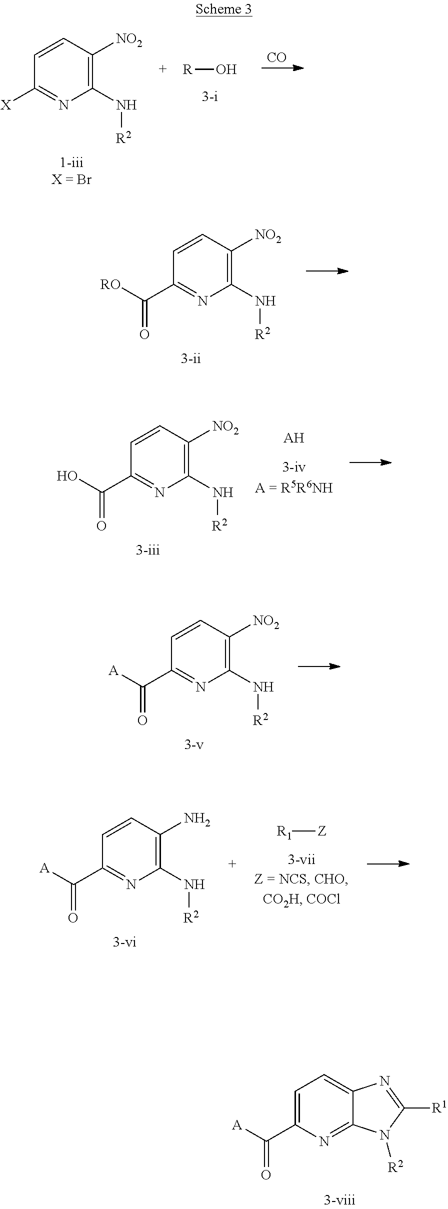

The intermediates 1-vi were used to prepare the compounds described herein as shown in the schemes below. In Scheme 4, compounds 4-ii described herein are prepared by treating intermediates 1-vi with amines 4-i in the presence of a carbonylation reagent such as Pd(PPh₃)₄ with carbon monoxide gas.

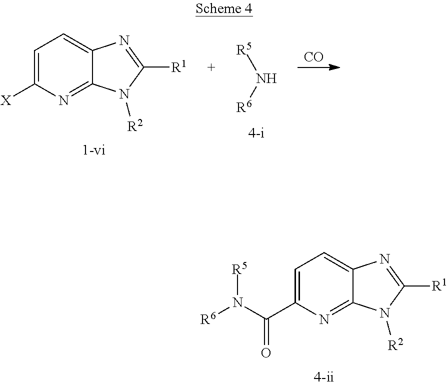

In Scheme 5, compounds 5-ii described herein are prepared by treating intermediates 1-vi with boronic acids 5-i under Suzuki coupling in conditions in the presence of a coupling agent such as Pd(PPh₃)₄.

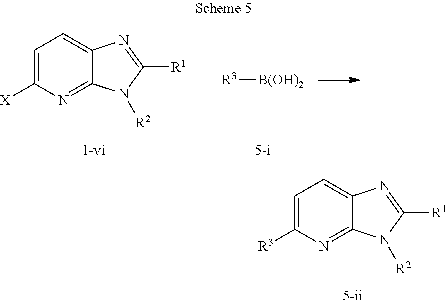

Scheme 6 describes a general synthetic approach to the compounds described herein. Compounds 6-viii are prepared by the following sequence. A dianilino compound such as 6-i (J. Med. Chem. 2007, 50, 5984-5993) is treated with electrophilic compounds 6-ii to provide intermediate 6-iii. Subsequent alkylation with electrophilic compounds 6-iv provides ester 6-v which, upon saponification and amide coupling with amine 6-vii, affords compounds 6-viii described herein.

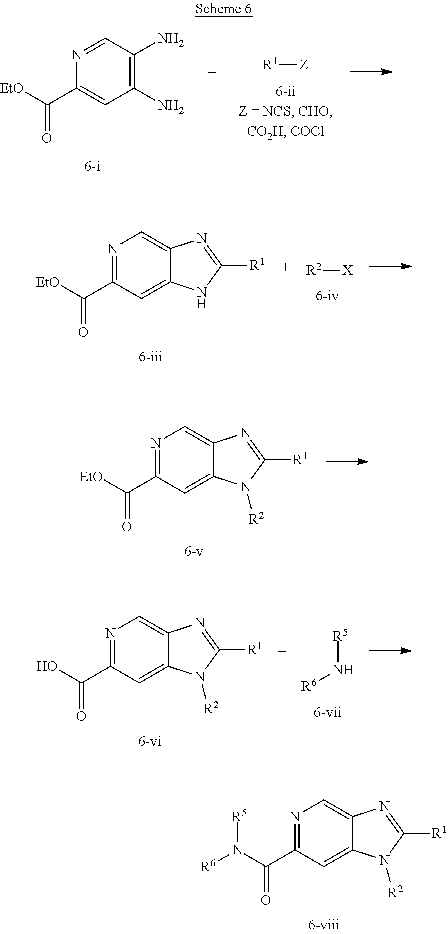

Alternatively, scheme 7 describes a modification of the general synthetic approach described in scheme 6 to the compounds described herein. Compounds 6-viii are prepared by the following sequence. Non-regioselective protection of intermediate 7-i with a suitable protecting group 7-ii, affords, after separation, esters 7-iii (for a related procedure see: WO2004/039803) which are saponified to intermediate 7-iv and coupled with amine 7-v to afford intermediates 7-vi. Upon treatment with an organolithium reagent, intermediates 7-vi are reacted with electrophilic compounds 7-vii to produce intermediate 7-viii. The compounds described herein 6-viii are finally obtained by treatment of deprotected intermediate 7-ix with electrophilic compound 7-x.

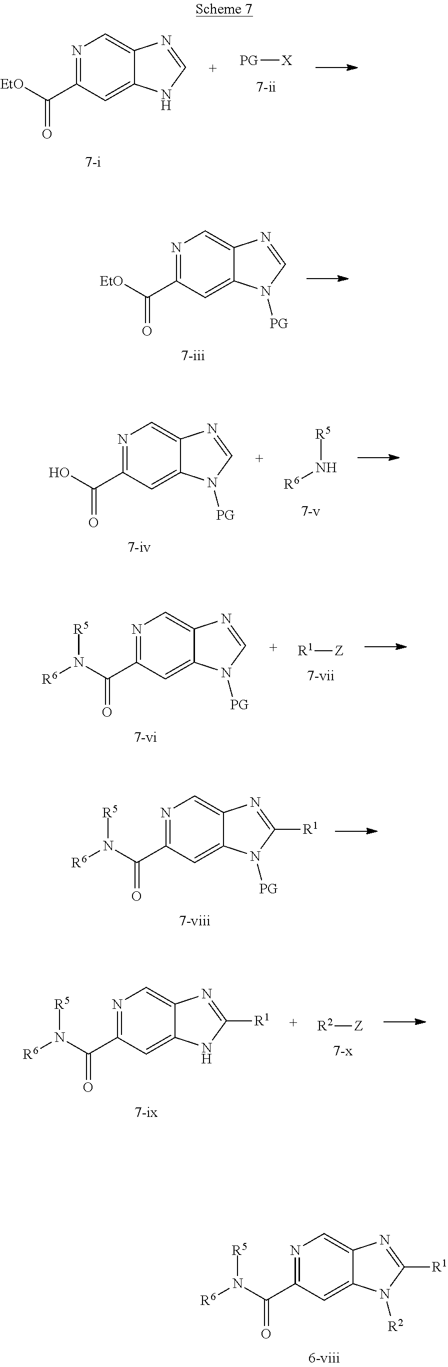

Scheme 8 describes another approach to the compounds of the present invention. Aminonicotinic acid 8-i is nitrated under standard conditions and the resulting nitro-pyridines 8-ii is esterified to 8-iii. The latter is then reduced to the diaminopyridines 8-iv which are transformed to imidazopyridines 8-vii using similar steps to that described above. A non-selective alkylation then affords the final compounds of the present invention, after chromatographic separation.

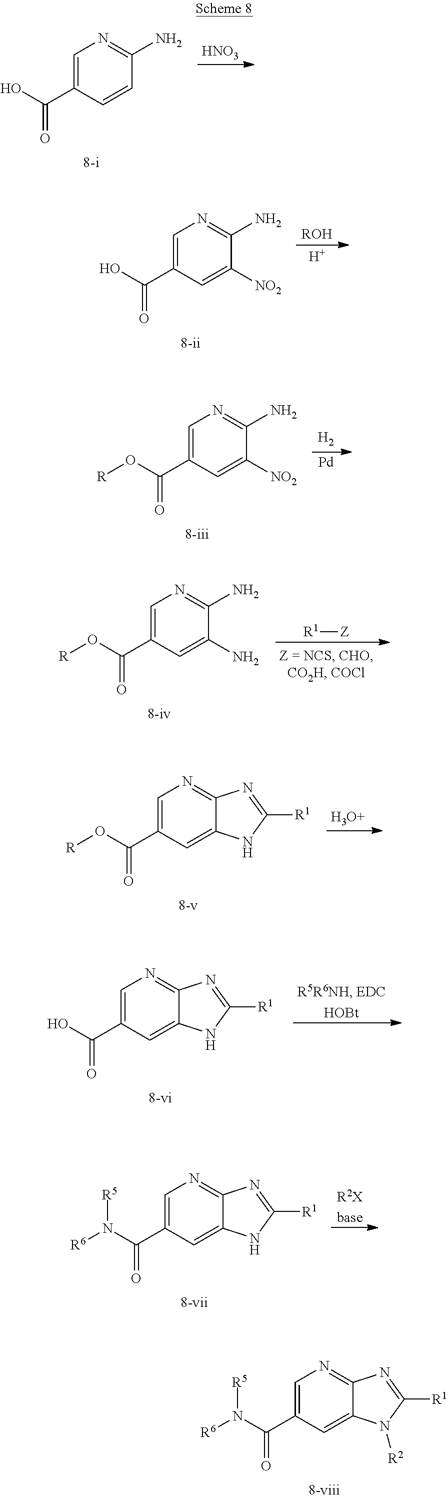

Scheme 9 describes a general synthetic approach to the compounds described herein. Compounds 9-i or 9-ii are prepared by the following sequence. Compounds 4-ii or compounds 6-viii or compounds 8-viii are treated with a suitable reducing agent to afford compounds 9-i or 9-ii described herein.

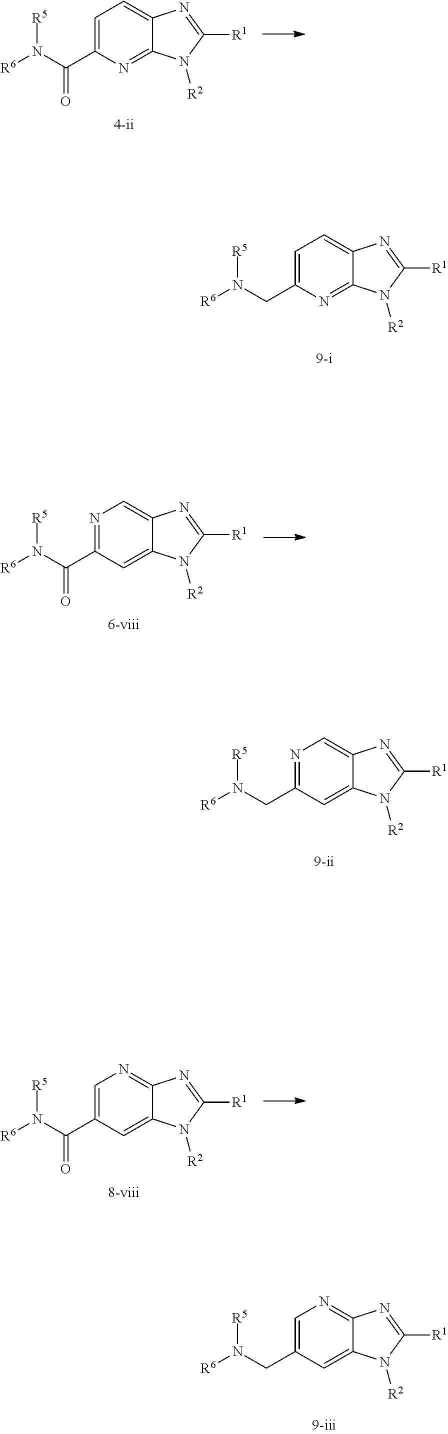

Scheme 10 describes a synthetic scheme to compounds with multiple nitrogens described herein. Dichloride 10-i is first treated with an amine to provide intermediate 10-ii which is treated with an aldehyde as described in Scheme 1 to afford 10-iii. Lithiation of 10-iii with a suitable alkyllithium reagent followed by the addition of an alkyl chloroformate provides the ester 10-iv which is hydrolyzed to the carboxylic acid 10-v using various method known in the art. Coupling with an amine under standard conditions affords 10-vi which is activated to a triflate with reagents such as trifluoromethanesulfonic anhydride and then reduced to the compounds of the present invention 10-vii.

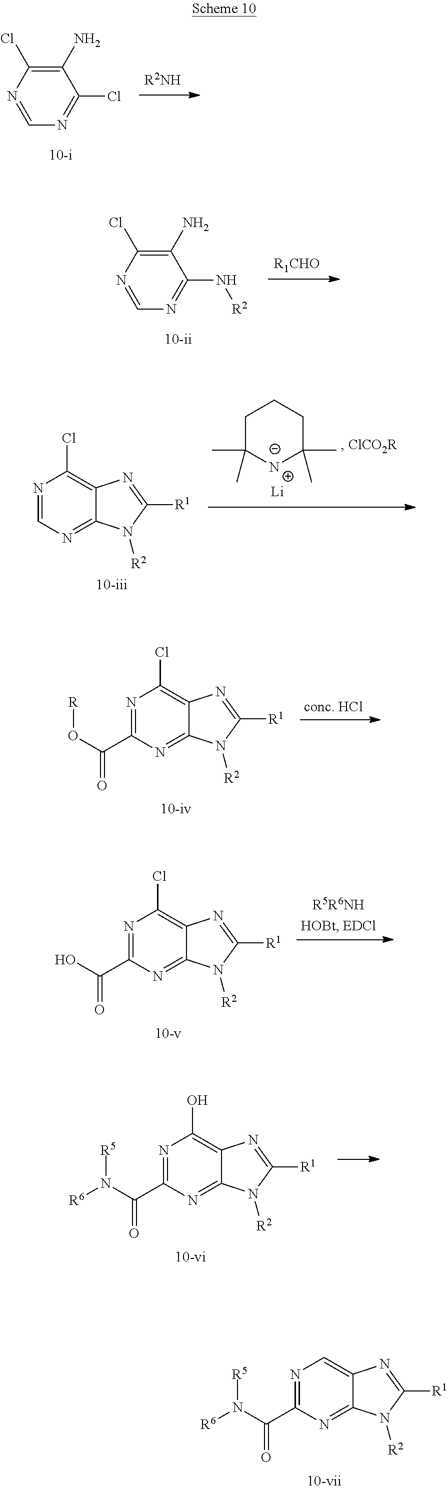

Scheme 11 describes a synthetic scheme to compounds with multiple nitrogens described herein. Dibromide 11-i is first treated with an amine to provide intermediate 11-ii using a procedure similar to that described in WO 08/016669 and Pharm. Chem. J. 1975, 3, 149. This diamino intermediate 11-ii is then converted to 11-iii which in turn is subjected to a carbonylation reaction to provide 11-iv.

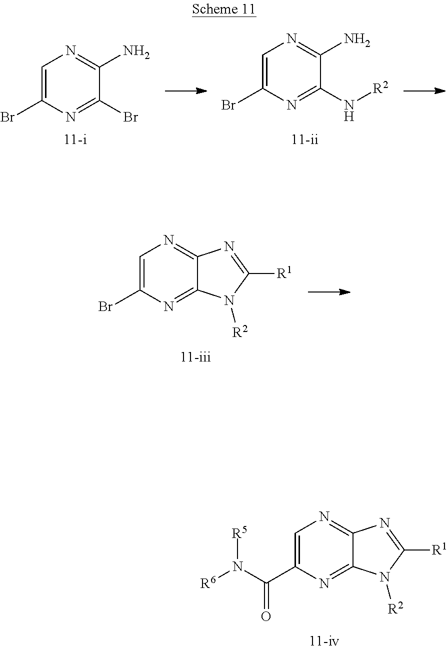

As described in Scheme 12, a subset of compounds described in Scheme 1 can be further activated to yield compounds of the present invention Thus the S-alkyl analogues 1-ix can be oxidized to their sulfone counterpart 12-i, which in turn afford compounds 12-ii when treated with a nucleophile reagent R¹H.

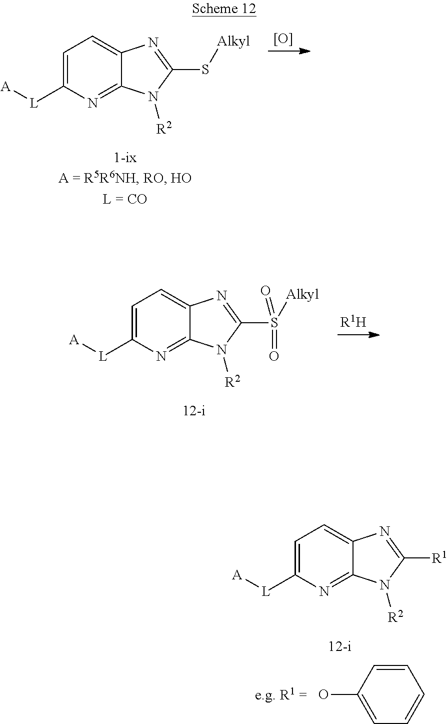

### General

Analytical Reverse-Phase-HPLC method:

### Solvent A: MeOH:H₂O:TFA (5:95:0.05)

### Solvent B: MeOH:H₂O:TFA (95:5:0.05)

Flow: 3.0 m/min.

### Gradient: 0% to 100% B in 2.0 min.

Column: ZorbaxC18, 3.5 microns, 4.6×30 mm

### Wavelength: 220 nm

LC-MS method:

### Solvent A: AcCN:H₂O:HCOOH (5:95:0.05)

### Solvent B: AcCN:H₂O:HCOOH (95:5:0.05)

### Gradient: 0% to 100% B in 2.0 min.

Flow: 0.3 mL/min
Column: ZorbaxC18, 3.5 microns, 2.1×30 mm

### Wavelength: 220 nm

### EXPERIMENTAL PROCEDURES

### Example 1

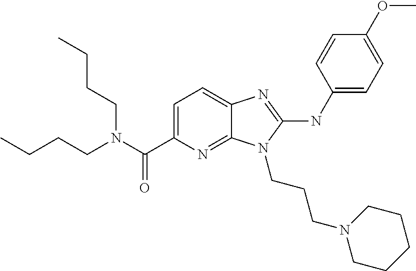 [SMILES: CCCCN(CCCC)C(=O)c1ccc2nc(Nc3ccc(OC)cc3)n(CCCN3CCCCC3)c2n1]

### N⁶,N⁶-dibutyl-2-((4-methoxyphenyl)amino)-3-(3-(piperidin-1-yl)propyl)-3H-imidazo[4,5-b]pyridine-5-carboxamide

### Intermediate 1A

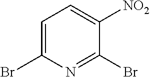 [SMILES: O=[N+]([O-])c1ccc(Br)nc1Br]

### 2,6-dibromo-3-nitropyridine

Intermediate 1A was prepared following the procedure described in JACS 2003, vol. 125, no. 19, p. 5707-5716

### Intermediate 1B

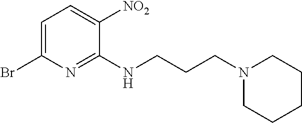 [SMILES: O=[N+]([O-])c1ccc(Br)nc1NCCCN1CCCCC1]

### 6-bromo-3-nitro-N-(3-(piperidin-1-yl)propyl)pyridin-2-amine

To a stirred solution of 2,6-dibromo-3-nitropyridine (1.7 g, 6.0 mmol) in acetonitrile (10 mL) at 0° C. was added dropwise a solution of 3-(piperidin-1-yl)propan-1-amine (1 mL, 6.0 mmol) in acetonitrile (10 mL). The mixture was allowed to warm to room temperature and stirred 3 days. The mixture was concentrated and the residue partitioned between ethyl acetate (25 mL) and a dilute sodium bicarbonate solution (25 mL). The separated aqueous layer was then extracted with ethyl acetate (2×25 mL). The combined ethyl acetate layers were washed with brine, dried over anhydrous Na₂SO₄, filtered and concentrated under reduced pressure to afford the title compound as a bright yellow solid (1.6 g, 78%). The material was used as is in the next step. LCMS m/z 345.1 (M+H)⁺, ret. time=3.79 min.

### Intermediate 1C

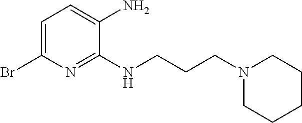 [SMILES: Nc1ccc(Br)nc1NCCCN1CCCCC1]

### 6-bromo-N-(3-(piperidin-1-yl)propyl)pyridine-2,3-diamine

To a stirred solution of 6-bromo-3-nitro-N-(3-(piperidin-1-yl)propyl)pyridin-2-amine (1.5 g, 4.4 mmol) in concentrated HCl (10 mL) at 0° C. was added portionwise SnCl₂-2H₂O (4.2 g, 18.4 mmol). The mixture was heated to 90° C. for 5 minutes then placed in an ice bath for 30 minutes. The mixture was basified with 15% NaOH (75 mL) and filtered trough a pad of Celite (1 inch thick) and washed with dichloromethane (100 ml). The filtrate layers were separated and the aqueous layer was further extracted with dichloromethane (2×100 mL). The combined organic layers were dried over anhydrous Na₂SO₄, filtered and concentrated under reduced pressure to afford the title compound as a black oil (1.4 g, quant.). The material was immediately used as is in the next step. LCMS m/z 313.1, 315.1 (M+H)⁺, ret. time=1.8 min.

### Intermediate 1D

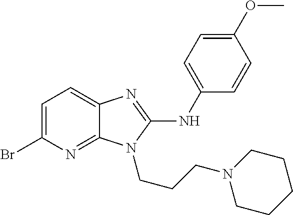 [SMILES: COc1ccc(Nc2nc3ccc(Br)nc3n2CCCN2CCCCC2)cc1]

### 5-bromo-N-(4-methoxyphenyl)-3-(3-(piperidin-1-yl)propyl)-3H-imidazo[4,5-b]pyridin-2-amine

To a stirred solution of 6-bromo-N²-(3-(piperidin-1-yl)propyl)pyridine-2,3-diamine (1.4 g, 4.4 mmol) in THF (10 mL) was added p-methoxyphenyl isothiocyanate (0.92 mL, 6.6 mmol) and N,N-dicyclohexylcarbodiimide (1.8 g, 8.8 mmol). The mixture was heated to 65° C. for 12 hours. The cooled mixture was poured into a saturated sodium bicarbonate solution (50 mL) and extracted with ethyl acetate (3×50 mL). The combined organic layers were washed with brine, dried over anhydrous Na₂SO₄, filtered and concentrated. The residue was purified by flash column chromatography eluting with 50% to 100% ethyl acetate in hexanes. The pooled fractions were concentrated under reduced pressure to afford the title compound as a white solid (1.5 g, 77%). LCMS m/z 344.2, 446.2 (M+H)⁺, ret. time=3.02 min.

### Intermediate 1E

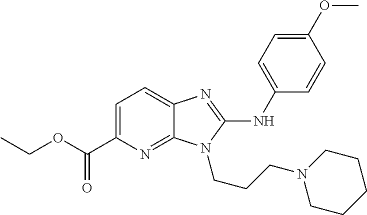 [SMILES: CCOC(=O)c1ccc2nc(Nc3ccc(OC)cc3)n(CCCN3CCCCC3)c2n1]

### Ethyl 2-((4-methoxyphenyl)amino)-3-(3-(piperidin-1-yl)propyl)-3H-imidazo[4,5-b]pyridine-5-carboxylate

A solution of 5-bromo-N-(4-methoxyphenyl)-3-(3-(piperidin-1-yl)propyl)-3H-imidazo[4,5-b]pyridin-2-amine (1.1 g, 2.5 mmol) in a mixture of triethylamine (7 mL) and anhydrous ethanol (20 mL) was purged with CO gas for 10 minutes. Bis(triphenylphosphine)palladium(II) dichloride (87 mg, 0.1 mmol) was added and the mixture was stirred at reflux under a CO atmosphere for 24 hours. More bis(triphenylphosphine)palladium(II) dichloride (87 mg, 0.1 mmol) was added and the mixture was stirred at reflux under a CO atmosphere for an extra 24 hours. The cooled mixture was concentrated under reduced pressure. The residue was partitioned between a saturated sodium bicarbonate solution (50 mL) and ethyl acetate (50 mL) then extracted with ethyl acetate (2×50 mL). The combined organic layers were washed with brine, dried over anhydrous Na₂SO₄, filtered and concentrated under reduced pressure. The residue was purified by flash column chromatography eluting with 50% to 100% ethyl acetate in hexanes then 0% to 15% methanol in dichloromethane. The pooled fractions were concentrated under reduced pressure to afford the title compound as a brown foam (0.7 g, 64%). LCMS m/z 438.3 (M+H)⁺, ret. time=3.04 min.

### Example 1

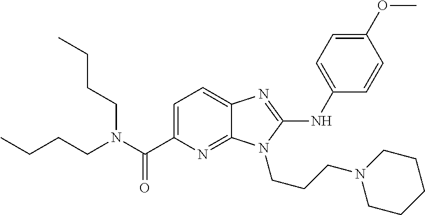 [SMILES: CCCCN(CCCC)C(=O)c1ccc2nc(Nc3ccc(OC)cc3)n(CCCN3CCCCC3)c2n1]

### N⁶,N⁶-dibutyl-2-((4-methoxyphenyl)amino)-3-(3-(piperidin-1-yl)propyl)-3H-imidazo[4,5-b]pyridine-5-carboxamide

To a stirred solution of di-n-butylamine (26 uL, 0.15 mmol) in toluene (2 mL) at 0° C. under nitrogen atmosphere was added a 2M trimethylaluminum solution in toluene (0.32 mL, 0.64 mmol). The mixture was stirred at room temperature for 45 min and added to a solution of ethyl 2-((4-methoxyphenyl)amino)-3-(3-(piperidin-1-yl)propyl)-3H-imidazo[4,5-b]pyridine-5-carboxylate (35 mg, 76 umol) in toluene (2 mL). The mixture was heated to 80° C. under a nitrogen atmosphere for 3 hours. The cooled mixture was poured on silica gel (3 g) slurried in dichloromethane (10 ml) and stirred 15 minutes. The slurry was filtered, washed with methanol (10 ml) and concentrated under reduced pressure. The residue was purified by reverse-phase preparative HPLC (Solvent A: MeOH:H₂O:TFA (5:95:0.05). Solvent B: MeOH:H₂O:TFA (95:5:0.05). Gradient 5 to 95% B in 15 min. Column: Zorbax SB-C18 PrepHT, 5 microns, 21.2×100 mm. Wavelength 220 nm) to afford the title compound as a yellow solid (14 mg, 35%). Analytical HPLC: ret. time=1.78 min; LCMS m/z 521.4 (M+H)⁺, ret. time=3.19 min. ¹H NMR (600 MHz, DMSO-d6) [symbol] ppm: 9.23 (1H, m), 7.69 (3H, m), 7.27 (1H, m), 7.00 (m, 2H), 4.31 (2H, m), 3.77 (3H, s), 3.41 (4H, m), 3.29 (2H, m), 3.11 (2H, m), 2.85 (2H, m), 2.17 (2H, m), 1.77 (2H, m), 1.67 (1H, m), 1.57 (6H, m), 1.36 (3H, m), 1.10 (2H, m), 0.93 (3H, m), 0.74 (3H, m).

### Example 2

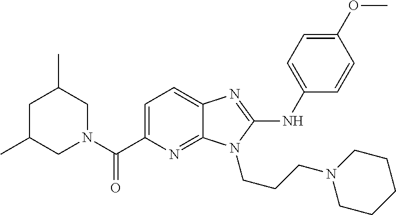 [SMILES: COc1ccc(Nc2nc3ccc(C(=O)N4CC(C)CC(C)C4)nc3n2CCCN2CCCCC2)cc1]

### (3,5-dimethylpiperidin-1-yl)(2-((4-methoxyphenyl)amino)-3-(3-(piperidin-1-yl)propyl)-3H-imidazo[4,5-b]pyridin-5-yl)methanone

### Intermediate 2A

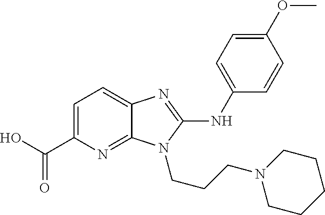 [SMILES: COc1ccc(Nc2nc3ccc(C(=O)O)nc3n2CCCN2CCCCC2)cc1]

### 2-((4-methoxyphenyl)amino)-3-(3-(piperidin-1-yl)propyl)-3H-imidazo[4,5-b]pyridine-5-carboxylic acid

A solution of ethyl 2-((4-methoxyphenyl)amino)-3-(3-(piperidin-1-yl)propyl)-3H-imidazo[4,5-b]pyridine-5-carboxylate (0.7 g, 1.6 mmol) in a one to one mixture of tetrahydrofuran and methanol (10 mL) was treated with a 2N LiOH aqueous solution (1.6 mL, 4.8 mmol) for 12 hours. The mixture was concentrated partially, diluted with water (15 mL) and washed with ethyl ether (15 mL) where a precipitate formed in the aqueous layer. The precipitate was filtered off to afford the title compound as a brown solid (0.41 g, 63%). LCMS m/z 410.2 (M+H)⁺, ret. time=1.1 min.

### Example 2

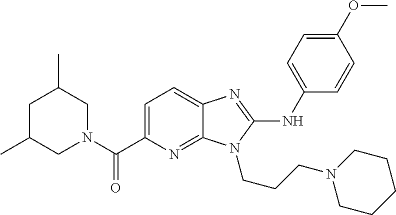 [SMILES: COc1ccc(Nc2nc3ccc(C(=O)N4CC(C)CC(C)C4)nc3n2CCCN2CCCCC2)cc1]

### (3,5-dimethylpiperidin-1-yl)(2-((4-methoxyphenyl)amino)-3-(3-(piperidin-1-yl)propyl)-3H-imidazo[4,5-b]pyridin-5-yl)methanone

To a stirred solution of 2-((4-methoxyphenyl)amino)-3-(3-(piperidin-1-yl)propyl)-3H-imidazo[4,5-b]pyridine-5-carboxylic acid (33 mg, 81 μmol), HOBt (19 mg, 0.14 mmol) and EDCI (26 mg, 0.14 mmol) in DMF (1 mL) was added 3,5-dimethylpiperidine (16 μL, 0.12 mmol). The mixture was stirred at room temperature for 12 hours and purified by reverse-phase preparative HPLC (Solvent A: MeOH:H₂O:TFA (5:95:0.05). Solvent B: MeOH:H₂O:TFA (95:5:0.05). Gradient 10 to 100% B in 20 min. Column: Zorbax SB-C18 PrepHT, 5 microns, 21.2×100 mm. Wavelength 220 nm) to afford the title compound as a white solid (18 mg, 46%). Analytical HPLC: ret. time=1.65 min; LCMS m/z 505.4 (M+H)⁺, ret. time=3.21 min. NMR (600 MHz, MeOD-d4) [symbol] ppm: 7.73 (1H, m), 7.47 (3H, m), 7.44 (1H, m), 7.09 (2H, m), 4.62 (1H, m), 4.41 (2H, m), 3.86 (3H, s), 3.70 (1H, m), 3.56 (1H, m), 3.26 (1H, m), 2.96 (2H, m), 2.66 (1H, m), 2.37 (4H, m), 2.06-1.65 (10H, m), 1.53 (1H, m), 1.02 (3H, d), 0.91 (1H, q), 0.81 (3H, d).

### Example 3 and Example 4

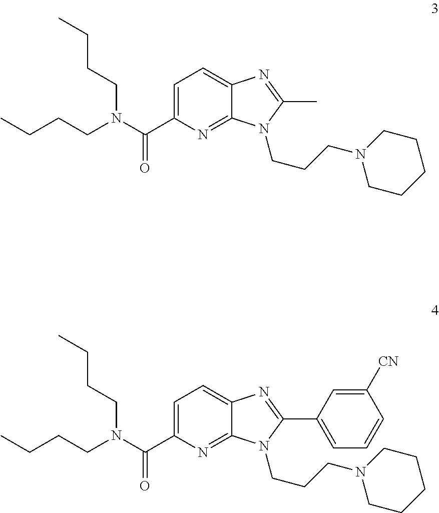 [SMILES: CCCCN(CCCC)C(=O)c1ccc2nc(C)n(CCCN3CCCCC3)c2n1 ; CCCCN(CCCC)C(=O)c1ccc2nc(-c3cccc(C#N)c3)n(CCCN3CCCCC3)c2n1]

### N,N-dibutyl-2-methyl-3-(3-(piperidin-1-yl)propyl)-3H-imidazo[4,5-b]pyridine-5-carboxamide (3) and N,N-dibutyl-2-(3-cyanophenyl)-3-(3-(piperidin-1-yl)propyl)-3H-imidazo[4,5-b]pyridine-5-carboxamide (4)

### Intermediate 3A

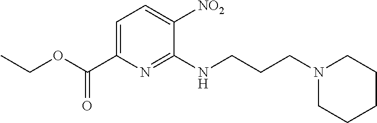 [SMILES: CCOC(=O)c1ccc([N+](=O)[O-])c(NCCCN2CCCCC2)n1]

### Ethyl 5-nitro-6-((3-(piperidin-1-yl)propyl)amino)picolinate

A solution of 6-bromo-3-nitro-N-(3-(piperidin-1-yl)propyl)pyridin-2-amine (1.5 g, 4.4 mmol) in a 1:3 mixture of triethylamine (7 mL) and ethanol (21 mL) was purged with CO gas for 10 minutes. Bis(triphenylphosphine)palladium(II) dichloride (154 mg, 0.2 mmol) was added and the mixture was stirred at reflux under a CO atmosphere (balloon) for 3 days. The cooled mixture was concentrated and purified by flash column chromatography eluting with 50% to 100% ethyl acetate in hexanes then 0% to 20% methanol in dichloromethane. The pooled fractions were concentrated under reduced pressure to afford the title compound as a yellow oil (0.66 g, 23%). LCMS m/z 337.2 (M+H)⁺, ret. time=2.9 min.

### Intermediate 3B

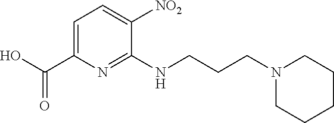 [SMILES: O=C(O)c1ccc([N+](=O)[O-])c(NCCCN2CCCCC2)n1]

### 5-nitro-6-((3-(piperidin-1-yl)propyl)amino)picolinic acid

A stirred solution of ethyl 5-nitro-6-((3-(piperidin-1-yl)propyl)amino)picolinate (0.66 g, 1.96 mmol) in a 1:1 mixture of tetrahydrofurane (5 mL) and methanol (5 mL) was treated with a 2N lithium hydroxide aqueous solution (2.9 mL, 5.9 mmol) for 12 hours. The mixture was concentrated under vacuo partially, diluted with water (10 mL) and washed with ethyl ether (10 mL). The aqueous layer was neutralized with a 1N hydrochloride aqueous solution and purified reverse-phase preparative HPLC (Solvent A: MeOH:H₂O:TFA (5:95:0.05). Solvent B: MeOH:H₂O:TFA (95:5:0.05). Gradient 10 to 100% B in 20 min. Column: Zorbax SB-C18 PrepHT, 5 microns, 21.2×100 mm. Wavelength 220 nm) to afford the title compound as a yellow foam (380 mg, 60%). LCMS m/z 309.2 (M+H)⁺, ret. time=0.78 min.

### Intermediate 3C

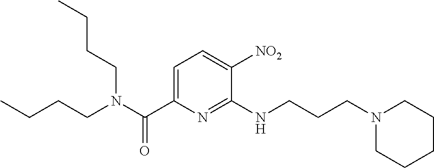 [SMILES: CCCCN(CCCC)C(=O)c1ccc([N+](=O)[O-])c(NCCCN2CCCCC2)n1]

### N,N-dibutyl-5-nitro-6-((3-(piperidin-1-yl)propyl)amino)picolinamide

To a stirred solution of 5-nitro-6-((3-(piperidin-1-yl)propyl)amino)picolinic acid (0.38 g, 1.2 mmol) in dimethylformamide (2 mL) was added HOBt (0.4 g, 2.9 mmol), EDCI (0.56 g, 2.9 mmol) and di-n-butylamine (0.4 mL, 2.4 mmol). The mixture was stirred at room temperature for 12 hours then diluted with ethyl acetate (75 mL) and washed with a saturated sodium bicarbonate solution, water twice then brine. The organic layer was dried over anhydrous Na₂SO₄, filtered and concentrated under reduced pressure to afford the title compound as a brown foam (0.5 g, quant.). LCMS m/z 420.3 (M+H)⁺, ret. time=3.33 min. Used as is in the next step.

### Intermediate 3D

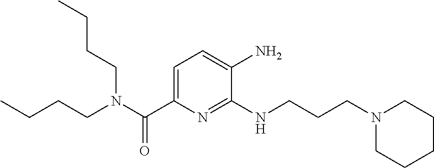 [SMILES: CCCCN(CCCC)C(=O)c1ccc(N)c(NCCCN2CCCCC2)n1]

### 5-amino-N,N-dibutyl-6-((3-(piperidin-1-yl)propyl)amino)picolinamide

To a stirred solution of N,N-dibutyl-5-nitro-6-((3-(piperidin-1-yl)propyl)amino)picolinamide (0.5 g, 1.2 mmol) in concentrated hydrochloric acid (3 mL) at 0° C. was added tin(II) chloride dihydrate (1.13 g, 5.0 mmol) portionwise. The suspension was heated to 90° C. for 5 minutes then cooled in an ice bath for 10 minutes. A 15% sodium hydroxide solution (25 mL) was added slowly. After stirring 10 minutes, the mixture was filtered trough a pad of Celite and washed with dichloromethane (30 mL). The filtrate was separated and the aqueous layer extracted with DCM (2×30 mL). The combined organic layers were dried over anhydrous Na₂SO₄, filtered and concentrated under vacuo to afford the title compound as a black oil (0.33 g, 70%). LCMS m/z 390.3 (M+H)⁺, ret. time=3.2 min. Used as is in the next step.

### Example 3 and Example 4

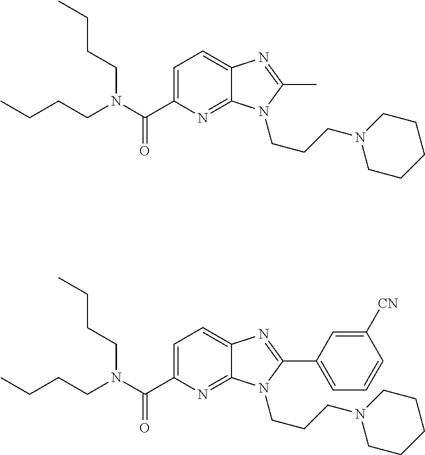 [SMILES: CCCCN(CCCC)C(=O)c1ccc2nc(C)n(CCCN3CCCCC3)c2n1 ; CCCCN(CCCC)C(=O)c1ccc2nc(-c3cccc(C#N)c3)n(CCCN3CCCCC3)c2n1]

### N,N-dibutyl-2-methyl-3-(3-(piperidin-1-yl)propyl)-3H-imidazo[4,5-b]pyridine-5-carboxamide (3) and N,N-dibutyl-2-(3-cyanophenyl)-3-(3-(piperidin-1-yl)propyl)-3H-imidazo[4,5-b]pyridine-5-carboxamide (4)

To a stirred solution of 5-amino-N,N-dibutyl-6-((3-(piperidin-1-yl)propyl)amino)picolinamide (54 mg, 0.14 mmol) in acetic acid (2 mL) was added 3-cyanobenzoyl chloride (23 mg, 0.14 mmol). The mixture was stirred at 100° C. for 12 hours then purified by reverse-phase preparative HPLC (Solvent A: MeOH:H₂O:TFA (5:95:0.05). Solvent B: MeOH:H₂O:TFA (95:5:0.05). Gradient 5 to 100% B in 20 min. Column: Zorbax SB-C18 PrepHT, 5 microns, 21.2×100 mm. Wavelength 220 nm) to afford the title compounds:

Example 3 as a dark oil (24 mg, 41%). Analytical HPLC: ret. time=1.72 min; LCMS m/z 414.3 (M+H)⁺, ret. time=3.2 min. NMR (600 MHz, MeOD-d4) δ ppm: 8.11 (1H, d), 7.50 (1H, d), 4.45 (2H, t), 3.52 (5H, m), 3.32 (1H, m), 3.22 (2H, m), 2.91 (2H, m), 2.78 (3H, s), 2.36, (2H, m), 1.95 (2H, m), 1.84 (1H, m), 1.77-1.69 (4H, m), 1.60 (2H, m), 1.53-1.39 (3H, m), 1.15 (2H, m), 1.02 (3H, t), 0.77 (3H, t).

Example 4 as a clear glass (12 mg, 17%). Analytical HPLC: ret. time=1.88 min; LCMS m/z 501.4 (M+H)⁺, ret. time=3.32 min. NMR (600 MHz, MeOD-d4) δ ppm: 8.24-8.21 (2H, m), 8.12 (1H, d), 8.03 (1H, d), 7.83 (1H, t), 7.54 (1H, d), 4.56 (2H, t), 3.58 (2H, t), 3.44 (2H, d), 3.36 (2H, t), 3.10 (2H, m), 2.86 (2H, t), 2.29 (2H, m), 1.89 (2H, d), 1.82 (1H, d), 1.77-1.58 (7H, m), 1.50-1.43 (3H, m), 1.18 (2H, m), 1.04 (3H, t), 0.80 (3H, t).

### Example 5

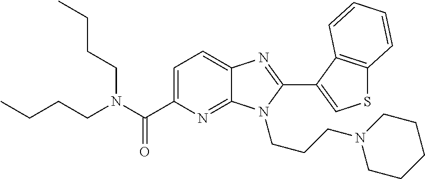 [SMILES: CCCCN(CCCC)C(=O)c1ccc2nc(-c3csc4ccccc34)n(CCCN3CCCCC3)c2n1]

### 2-(benzo[b]thiophen-3-yl)-N,N-dibutyl-3-(3-(piperidin-1-yl)propyl)-3H-imidazo[4,5-b]pyridine-5-carboxamide

To a stirred solution of 5-amino-N,N-dibutyl-6-((3-(piperidin-1-yl)propyl)amino)picolinamide (54 mg, 0.14 mmol) in nitrobenzene (2 mL) was added benzothiophene-3-carboxaldehyde (30 mg, 0.18 mmol). The mixture was stirred at 100° C. for 12 hours then purified by reverse-phase preparative HPLC (Solvent A: MeOH:H₂O:TFA (5:95:0.05). Solvent B: MeOH:H₂O:TFA (95:5:0.05). Gradient 30 to 100% B in 17 min. Column: Zorbax SB-C18 PrepHT, 5 microns, 21.2×100 mm. Wavelength 220 nm) to afford the title compound (42 mg, 40%). Analytical HPLC: ret. time=2.01 min; LCMS m/z 532.3 (M+H)⁺, ret. time=3.43 min. NMR (600 MHz, MeOH-d4) δ ppm: 8.31 (1H, s), 8.28 (1H, d), 8.09 (1H, m), 8.04 (1H, m), 7.59 (1H, d), 7.53 (1H, m), 4.57 (2H, t), 3.59 (2H, m), 3.39 (2H, m), 3.34 (1H, m), 3.00 (2H, m), 2.75 (2H, m), 2.22 (2H, m), 1.85 (2H, m), 1.75 (2H, m), 1.68-1.56 (4H, m), 1.51-1.36 (3H, m), 1.19 (2H, m), 1.04 (3H, t), 0.82 (3H, t).

### Example 36

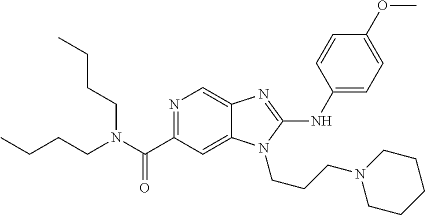 [SMILES: CCCCN(CCCC)C(=O)c1cc2c(cn1)nc(Nc1ccc(OC)cc1)n2CCCN1CCCCC1]

### N,N-dibutyl-2-((4-methoxyphenyl)amino)-1-(3-(piperidin-1-yl)propyl)-1H-imidazo[4,5-c]pyridine-6-carboxamide

### Intermediate 36A

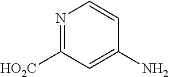

### 4-aminopicolinic acid

A thick suspension of Picloram (8 g, 33 mmol) and 10% Pd/C (1.2 g) in a 10% LiOH aqueous solution (44 mL) was purged with hydrogen gas twice then stirred under a hydrogen atmosphere (45 PSI) at 40° C. for 4 hours. The mixture was then heated to 70° C. for 12 hours. The cooled suspension was filtered on Celite. The filtrate was made acidic (pH=3) with concentrated HCl (app. 3.5 mL) at which point a precipitate formed. The solid was filtered off and dried under Hi-Vac overnight to afford the title compound as a beige solid (4.6 g, 99%). LCMS m/z 139.1 (M+H)⁺, ret. time=0.21 min.

### Intermediate 36B

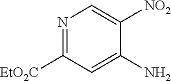 [SMILES: CCOC(=O)c1cc(N)c([N+](=O)[O-])cn1]

### Ethyl 4-amino-5-nitropicolinate

To a cooled solution of 4-aminopicolinic (4.6 g, 33.3 mmol) in concentrated H₂SO₄ (30 mL) was added KNO₃ (3.4 g, 33.3 mmol). The mixture was warmed to room temperature for 20 minutes then heated to 75° C. for 2 hours. To the cooled mixture (ice bath) was added ice-cold EtOH (90 mL) slowly. The resulting mixture was heated to 60° C. for 12 hours. The cooled mixture was slowly poured into an ice-cold solution of potassium acetate (120 g) in water (225 mL) resulting in coprecipitation of product with K₂SO₄. Filtered and washed solid with cold water (500 mL). The dried beige solid (8.3 g) was triturated with THF (3×250 mL) and the combined filtrates were concentrated to afford the title compound as a beige solid (3.7 g, 53%). LCMS m/z 212.1 (M+H)⁺, ret. time=1.82 min.

### Intermediate 36C

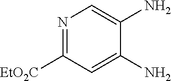 [SMILES: CCOC(=O)c1cc(N)c(N)cn1]

### Ethyl 4,5-diaminopicolinate

A 35 mL pressure vessel was charged with ethyl 4-amino-5-nitropicolinate (2.7 g, 12.8 mmol), 10% Pd/C (0.54 g, Degussa type) and a 1:5 mixture (30 mL) of 4-methylcycolhexene and propan-2-ol. The flask was sealed and heated to 100° C. for 12 h. The cooled mixture was filtered on Celite and washed with ethyl acetate (100 mL). The filtrate was concentrated to a brown oil, redissolved in DCM (10 mL) and hexanes (60 mL) and evaporated under vaccuo with heating to afford the title compound as a grey solid (2.3 g, 100%). Used as is in the next step. LCMS m/z 182.1 (M+H)⁺, ret. time=0.24 min.

### Intermediate 36D

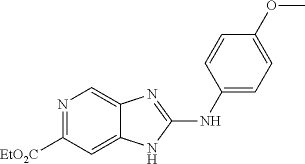 [SMILES: CCOC(=O)c1cc2[nH]c(Nc3ccc(OC)cc3)nc2cn1]

### Ethyl 2-((4-methoxyphenyl)amino)-1H-imidazo[4,5-c]pyridine-6-carboxylate

To a stirred solution of ethyl 4,5-diaminopicolinate (2.3 g, 12.8 mmol) in THF (130 mL) was added p-methoxyphenyl isothiocyanate (3.1 mL, 22.1 mmol) and DCC (4.8 g, 23.0 mmol). The mixture was stirred at 65° C. for 12 h. The cooled mixture was diluted with a 1N aqueous HCl solution (200 mL) and ethyl acetate (200 mL) and stirred vigorously for 15 minutes. A white solid was filtered off (dicyclohexylthiourea) and the separated organic layer of the filtrate was extracted further with a 1N aqueous HCl solution (75 mL). The combined acid aqueous layers were made basic with concentrated NH₄OH solution (pH=10). A precipitate was filtered off and dried under Hi-Vac overnight to afford the title compound as a beige solid (2.5 g, 63%). LCMS m/z 313.1 (M+H)⁺, ret. time=1.86 min.

### Intermediate 36E

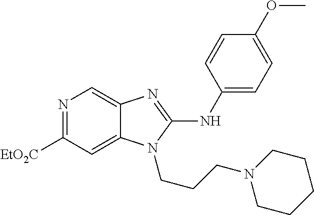 [SMILES: CCOC(=O)c1cc2c(cn1)nc(Nc1ccc(OC)cc1)n2CCCN1CCCCC1]

### Ethyl 2-((4-methoxyphenyl)amino)-1-(3-(piperidin-1-yl)propyl)-1H-imidazo[4,5-c]pyridine-6-carboxylate

To a stirred suspension of ethyl 2-((4-methoxyphenyl)amino)-1H-imidazo[4,5-c]pyridine-6-carboxylate (0.5 g, 1.6 mmol) and K₂CO₃ (0.7 g, 4.8 mmol) in DMF (10 mL) was added 1-(3-chloropropyl)piperidine hydrochloride (0.28 g, 1.9 mmol). The suspension was stirred at 65° C. for 12 h. The mixture was poured into ethyl acetate (50 mL), washed with water (3×10 mL) and brine (10 mL). The organic layer was dried over Na₂SO₄, filtered and concentrated to an oil. The residue was purified by reverse-phase preparative HPLC (Solvent A: MeOH:HO₂O:TFA (5:95:0.05). Solvent B: MeOH:H₂O:TFA (95:5:0.05). Gradient 20 to 70% B in 15 min. Column: Zorbax SB-C18 PrepHT, 5 microns, 21.2×100 mm. Wavelength 220 nm) to afford the title compounds as a yellow solid (0.35 g, 33%). LCMS m/z 438.3 (M+H)⁺, ret. time=1.60 min.

### Intermediate 36F

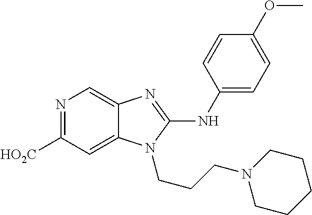 [SMILES: COc1ccc(Nc2nc3cnc(C(=O)O)cc3n2CCCN2CCCCC2)cc1]

### 2-((4-methoxyphenyl)amino)-1-(3-(piperidin-1-yl)propyl)-1H-imidazo[4,5-c]pyridine-6-carboxylic acid

A stirred solution of ethyl 2-((4-methoxyphenyl)amino)-1-(3-(piperidin-1-yl)propyl)-1H-imidazo[4,5-c]pyridine-6-carboxylate (90 mg, 0.21 mmol) in concentrated HCl (20 mL) was stirred at 70° C. for 12 h. The mixture was concentrated to afford the title compound as a beige solid (90 mg, quant.). LCMS m/z 410.2 (M+H)⁺, ret. time=0.36 min.

### Example 36

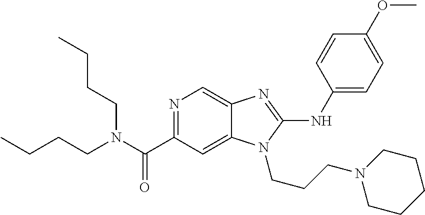 [SMILES: CCCCN(CCCC)C(=O)c1cc2c(cn1)nc(Nc1ccc(OC)cc1)n2CCCN1CCCCC1]

### N,N-dibutyl-2-((4-methoxyphenyl)amino)-1-(3-(piperidin-1-yl)propyl)-1H-imidazo[4,5-c]pyridine-6-carboxamide

To a stirred solution of 2-((4-methoxyphenyl)amino)-1-(3-(piperidin-1-yl)propyl)-1H-imidazo[4,5-c]pyridine-6-carboxylic acid (90 mg, 0.21 mmol) in DMF (2 mL) was added Et₃N (87 μL, 0.61 mmol), EDCI (69 mg, 0.36 mmol), HOBt (49 mg, 0.36 mmol) and di-n-butylamine (61 μL, 0.36 mL). The mixture was stirred 12 hours then purified by reverse-phase preparative HPLC (Solvent A: MeOH:H₂O:TFA (5:95:0.05). Solvent B: MeOH:H₂O:TFA (95:5:0.05). Gradient 40 to 100% B in 15 min. Column: Zorbax SB-C18 PrepHT, 5 microns, 21.2×100 mm. Wavelength 220 nm) to afford the title compounds as a white solid (30 mg, 19%). LCMS m/z 521.4 (M+H)⁺, ret. time=2.15 min.

### Example 37

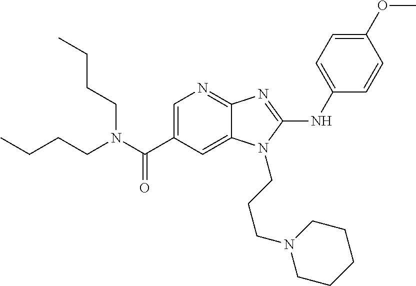 [SMILES: CCCCN(CCCC)C(=O)c1cnc2nc(Nc3ccc(OC)cc3)n(CCCN3CCCCC3)c2c1]

### N,N-dibutyl-2-((4-methoxyphenyl)amino)-1-(3-(piperidin-1-yl)propyl)-1H-imidazo[4,5-b]pyridine-6-carboxamide

### Intermediate 37A

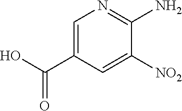 [SMILES: Nc1ncc(C(=O)O)cc1[N+](=O)[O-]]

### 6-amino-5-nitronicotinic acid

To a stirred suspension of 6-aminonicotinic acid (6.5 g, 47.0 mmol) in concentrated sulfuric acid (13 mL) was added at 0° C. dropwise over 20 minutes a 1:1 mixture of nitric acid and sulphuric acid (6.5 mL). The mixture was stirred at room temperature for 1.5 h and the resulting orange slurry was poured into a mixture of ice and water (450 mL). The resulting solid was filtered and dried under vacuum overnight to afford a yellow solid (7.6 g). This solid was resuspended in concentrated sulphuric acid (25 mL) and heated to 100° C. for 1.5 h. The red suspension was cooled to 0° C. and treated with 1N NaOH to adjust to pH=2 where a yellow precipitate formed. The resulting solid was filtered and dried under vacuum overnight to afford a yellow solid (7.5 g). LCMS m/z 184.0 (M+H)⁺, ret. time=0.65 min.

### Intermediate 37B

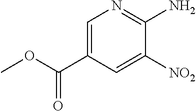

### methyl 6-amino-5-nitronicotinate

To a stirred suspension of 6-amino-5-nitronicotinic acid (7.5 g, 41.0 mmol) in methanol (150 mL) was slowly added concentrated sulphuric acid (16 mL) at 0° C. The mixture was heated to 60° C. and stirred at 60° C. for 5 hours at which time the mixture was cooled and concentrated under vacuum. The residue was neutralized slowly with saturated sodium bicarbonate and the resulting precipitate was filtered and dried under vacuum overnight to afford a yellow solid (3.5 g). LCMS m/z 198.0 (M+H)+, ret. time=2.10 min.

### Intermediate 37C

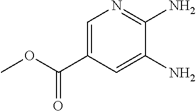

### methyl 5,6-diaminonicotinate

A stirred suspension of methyl 5-amino-6-nitronicotinate (3.5 g, 17.7 mmol) and 50% w/w Raney Nickel/water (7 g) in methanol (200 mL) was heated to 80° C. under 40 p.s.i. of hydrogen for 5 hours. Upon cooling, the hydrogen was evacuated and the mixture was filtered through Celite. Concentration afforded a beige solid (2.7 g). LCMS m/z 168.8 (M+H)⁺, ret. time=0.20 min.

### Intermediate 37D

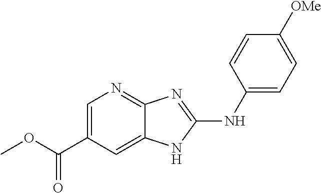 [SMILES: COC(=O)c1cnc2nc(Nc3ccc(OC)cc3)[nH]c2c1]

### methyl 2-((4-methoxyphenyl)amino)-1H-imidazo[4,5-b]pyridine-6-carboxylate

The title compound was prepared from methyl 5,6-diaminonicotinate using the procedure described for the preparation of Intermediate 1D. LCMS m/z 299.1 (M+H)⁺, ret. time=1.96 min.

### Intermediate 37E

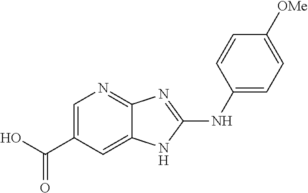 [SMILES: COc1ccc(Nc2nc3ncc(C(=O)O)cc3[nH]2)cc1]

### 2-((4-methoxyphenyl)amino)-1H-imidazo[4,5-b]pyridine-6-carboxylic acid

A solution of methyl 2-((4-methoxyphenyl)amino)-1H-imidazo[4,5-b]pyridine-6-carboxylate (220 mg, 0.74 mmol) in concentrated HCl was stirred at 70° C. for 4 hours. Upon cooling, the mixture was concentrated under reduced pressure and dried under vacuum to afford a yellow solid. LCMS m/z 285.2 (M+H)⁺, ret. time=1.71 min.

### Intermediate 37F

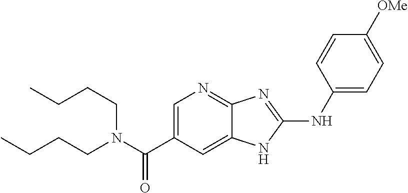 [SMILES: CCCCN(CCCC)C(=O)c1cnc2nc(Nc3ccc(OC)cc3)[nH]c2c1]

### N,N-dibutyl-2-((4-methoxyphenyl)amino)-1H-imidazo[4,5-b]pyridine-6-carboxamide

The title compound was prepared from methyl 2-((4-methoxyphenyl)amino)-1H-imidazo[4,5-b]pyridine-6-carboxylic acid and dibutylamine using a procedure similar to the one described for the preparation of Example 2. The reaction was carried out at 65° C. LCMS m/z 396.2 (M+H)⁺, ret. time=2.26 min.

### Example 37

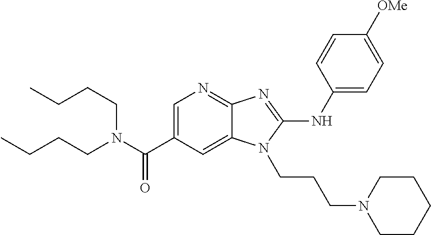 [SMILES: CCCCN(CCCC)C(=O)c1cnc2nc(Nc3ccc(OC)cc3)n(CCCN3CCCCC3)c2c1]

### N,N-dibutyl-2-((4-methoxyphenyl)amino)-1-(3-(piperidin-1-yl)propyl)-1H-imidazo[4,5-b]pyridine-6-carboxamide

To a stirred suspension of N,N-dibutyl-2-((4-methoxyphenyl)amino)-1H-imidazo[4,5-b]pyridine-6-carboxamide (120 mg, 0.3 mmol) in DMF (1.0 mL), was added a 1.0 M solution of lithium bis(trimethylsilyl)amide (0.31 mL, 0.31 mmol). The resulting mixture was stirred at room temperature for 30 minutes and a solution of 1-(3-chloropropyl)piperidine hydrochloride (60 mg, 0.30 mmol) and lithium bis(trimethylsilyl)amide (0.31 mL, 0.31 mmol). in DMF (1.0 mL) was added. The resulting mixture was stirred at 60° C. for 14 hours. The mixture was allowed to cool to room temperature and trifluoroacetic acid was added. The mixture was filtered and purified by reverse-phase preparative HPLC (Solvent A: MeOH:H₂O:TFA (5:95:0.05). Solvent B: MeOH:H₂O:TFA (95:5:0.05). Gradient 30 to 100% B in 12 min. Column: Zorbax SB-C18 PrepHT, 5 microns, 21.2×100 mm. Wavelength 220 nm). LCMS m/z 521.4 (M+H)⁺, ret. time=2.15 min.

### Example 46

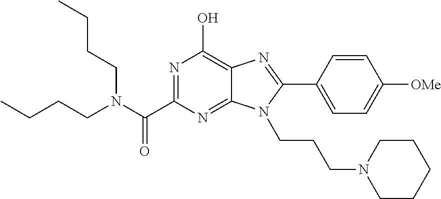 [SMILES: CCCCN(CCCC)C(=O)c1nc(O)c2nc(-c3ccc(OC)cc3)n(CCCN3CCCCC3)c2n1]

### 6-hydroxy-N,N-diisopentyl-8-(4-methoxyphenyl)-9-(3-(piperidin-1-yl)propyl)-9H-purine-2-carboxamide

### Intermediate 46A

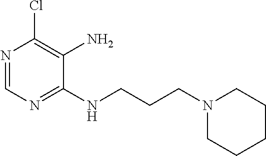 [SMILES: Nc1c(Cl)ncnc1NCCCN1CCCCC1]

### 6-chloro-N⁴-(3-(piperidin-1-yl)propyl)pyrimidine-4,5-diamine

To a stirred solution of 5-amino-4,6-dichloropyrimidine (1.0 g, 6.1 mmol) in n-butanol (10 mL) was added 3-(piperidin-1-yl)propan-1-amine (1.3 g, 6.4 mmol) and triethylamine (1.9 mL, 13.4 mmol). The mixture was stirred at 80° C. for 12 hours, cool down and evaporated under reduced pressure. The residue was dissolved in dichloromethane and a 5% solution of ammonium hydroxide was added. The separated aqueous layer was extracted with dichloromethane (3×30 mL) and the combined organic layers were washed with brine, dried (sodium sulphate), filtered and evaporated to yield the title compound as an orange oil. LCMS m/z 270.1 (M+H)⁺, ret. time=0.26 min.

### Intermediate 46B

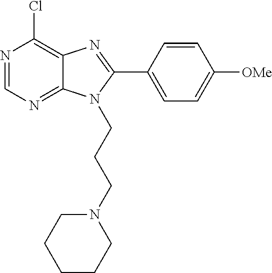 [SMILES: COc1ccc(-c2nc3c(Cl)ncnc3n2CCCN2CCCCC2)cc1]

### 6-chloro-8-(4-methoxyphenyl)-9-(3-(piperidin-1-yl)propyl)-9H-purine

The title compound was prepared from 6-chloro-N⁴-(3-(piperidin-1-yl)propyl)pyrimidine-4,5-diamine and 4-methoxybenzaldehyde using a procedure similar to the one described for the preparation of Example 5. LCMS m/z 386.2 (M+H)⁺, ret. time=1.86 min.

### Intermediate 46C

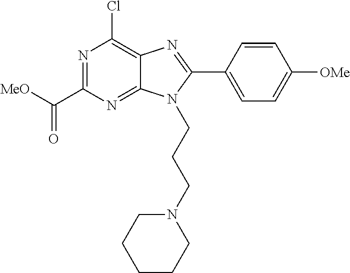 [SMILES: COC(=O)c1nc(Cl)c2nc(-c3ccc(OC)cc3)n(CCCN3CCCCC3)c2n1]

### methyl 6-chloro-8-(4-methoxyphenyl)-9-(3-(piperidin-1-yl)propyl)-9H-purine-2-carboxylate

To a stirred solution of 2,2,6,6-tetramethylpiperidine (0.44 mL, 2.6 mmol) in THF (1.0 mL) at −78° C. was added n-butyllithium (1.6 mL, 1.6 M, 2.6 mmol) and the mixture was warmed to room temperature. The resulting solution was added to a solution of 6-chloro-8-(4-methoxyphenyl)-9-(3-(piperidin-1-yl)propyl)-9H-purine (0.2 g, 0.5 mmol) in THF at 78° C. The mixture was stirred at 78° C. for 5 minutes and methyl chloroformate (0.38 mL, 5.0 mmol) was added. The mixture was stirred at 78° C. for 5 minutes and a saturated solution of ammonium chloride (5 mL) was added. The separated aqueous layer was extracted with ethyl acetate (3×30 mL) and the combined organic layers were washed with brine, dried (sodium sulphate), filtered and evaporated. The residue was purified by reverse-phase preparative HPLC (Solvent A: MeOH:H₂O:TFA (5:95:0.05). Solvent B: MeOH:H₂O:TFA (95:5:0.05). Gradient 10 to 75% B in 13 min. Column: Zorbax SB-C18 PrepHT, 5 microns, 21.2×100 mm. Wavelength 220 nm). LCMS m/z 444.2 (M+H)⁺, ret. time=1.98 min.

### Intermediate 46D

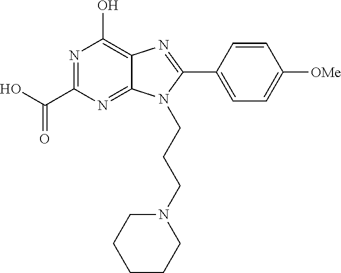 [SMILES: COc1ccc(-c2nc3c(O)nc(C(=O)O)nc3n2CCCN2CCCCC2)cc1]

### 6-hydroxy-8-(4-methoxyphenyl)-9-(3-(piperidin-1-yl)propyl)-9H-purine-2-carboxylic acid

The title compound was prepared from methyl 6-chloro-8-(4-methoxyphenyl)-9-(3-(piperidin-1-yl)propyl)-9H-purine-2-carboxylate using the procedure described for the preparation of Intermediate 45E. LCMS m/z 412.2 (M+H)⁺, ret. time=1.82 min.

### Example 46

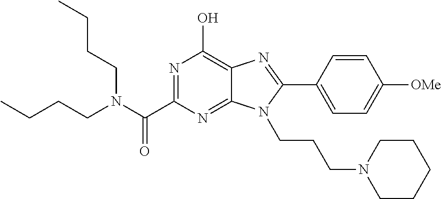 [SMILES: CCCCN(CCCC)C(=O)c1nc(O)c2nc(-c3ccc(OC)cc3)n(CCCN3CCCCC3)c2n1]

### 6-hydroxy-N,N-diisopentyl-8-(4-methoxyphenyl)-9-(3-(piperidin-1-yl)propyl)-9H-purine-2-carboxamide

The title compound was prepared from 6-hydroxy-8-(4-methoxyphenyl)-9-(3-(piperidin-1-yl)propyl)-9H-purine-2-carboxylic acid and diisopentylamine according to the procedure described for the preparation of Example 2. The reaction was carried out at 65° C. LCMS m/z 523.3 (M+H)⁺, ret. time=2.16 min.

### Example 49

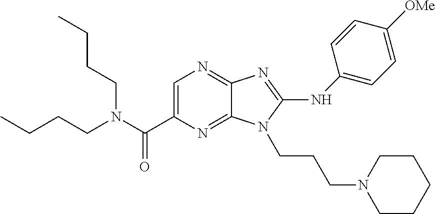 [SMILES: CCCCN(CCCC)C(=O)c1cnc2nc(Nc3ccc(OC)cc3)n(CCCN3CCCCC3)c2n1]

### N,N-dibutyl-2-((4-methoxyphenyl)amino)-1-(3-(piperidin-1-yl)propyl)-1H-imidazo[4,5-b]pyrazine-6-carboxamide

### Intermediate 49A

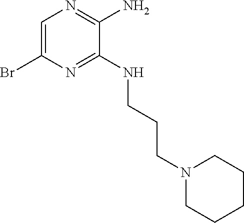 [SMILES: Nc1ncc(Br)nc1NCCCN1CCCCC1]

### 6-bromo-M-(3-(piperidin-1-yl)propyl)pyrazine-2,3-diamine

A suspension of 2,6-dibromopyrazin-3-amine (0.1 g, 0.4 mmol) in 3-(piperidin-1-yl)propan-1-amine (0.2 mL) was heated to 150° C. for 30 minutes in a microwave reactor. The mixture was purified by reverse-phase preparative HPLC (Solvent A: MeOH:H₂O:TFA (5:95:0.05). Solvent B:MeOH:H₂O:TFA (95:5:0.05). Gradient 10 to 70% B in 10 min. Column: Zorbax SB-C18 PrepHT, 5 microns, 21.2×100 mm. Wavelength 220 nm). LCMS m/z 314.1, 316.1 (M+H)⁺, ret. time=1.66 min.

### Intermediate 49B

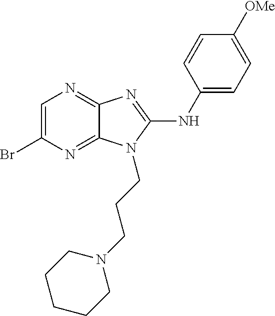 [SMILES: COc1ccc(Nc2nc3ncc(Br)nc3n2CCCN2CCCCC2)cc1]

### 6-bromo-N-(4-methoxyphenyl)-1-(3-(piperidin-1-yl)propyl)-1H-imidazo[4,5-b]pyrazin-2-amine

The title compound was prepared from 6-bromo-M-(3-(piperidin-1-yl)propyl)pyrazine-2,3-diamine and 4-methoxyphenyl isothiocyanate according to the procedure described for the preparation of Intermediate 1D. LCMS m/z 445.1, 447.1 (M+H)⁺, ret. time=2.03 min.

### Example 49

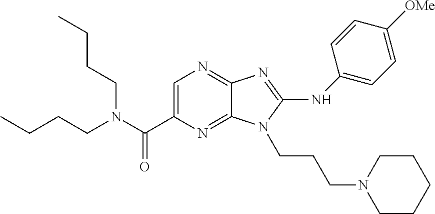 [SMILES: CCCCN(CCCC)C(=O)c1cnc2nc(Nc3ccc(OC)cc3)n(CCCN3CCCCC3)c2n1]

### N,N-dibutyl-2-((4-methoxyphenyl)amino)-1-(3-(piperidin-1-yl)propyl)-1H-imidazo[4,5-b]pyrazine-6-carboxamide

In a 50 mL sealed tube were added 6-bromo-N-(4-methoxyphenyl)-1-(3-(piperidin-1-yl)propyl)-1 H-imidazo[4,5-b]pyrazin-2-amine (44 mg, 0.1 mmol), dibutylamine (75 μl, 0.4 mmol), and triethylamine (1.0 mL) in toluene (4.0 mL) and carbon monoxide was bubbled through the solution for 5 minutes. Palladium dichloridebis(triphenylphosphine) (9.0 mg, 0.013 mmol) was added to give a tan suspension and carbon monoxide was bubbled through the mixture for an additional 5 minutes. The mixture was sealed and heated to 100° C. for 12 hours. Upon cooling, the mixture was concentrated, filtered and purified by reverse-phase preparative HPLC (Solvent A: MeOH:H₂O:TFA (5:95:0.05). Solvent B: MeOH:H₂O:TFA (95:5:0.05). Gradient 30 to 100% B in 20 min. Column: Sunfire Prep C18 ODB, 5 microns, 19×150 mm. Wavelength 220 nm). LCMS m/z 522.4 (M+H)⁺, ret. time=2.28 min.

The above procedure can be used to prepared the amide compounds described in the Table by replacing dibutylamine by other amines. Most amines used are from commercial sources except bis(2-cyclopropylethyl)amine used to prepare Example 37 In this case, the required bis(2-cyclopropylethyl)amine was prepared according to the reaction sequence described in patent application WO08116665 page 84.

### Example 53

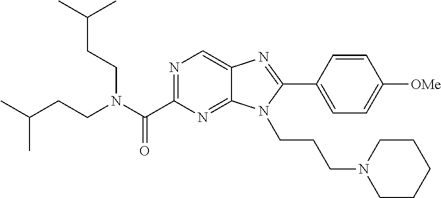 [SMILES: COc1ccc(-c2nc3cnc(C(=O)N(CCC(C)C)CCC(C)C)nc3n2CCCN2CCCCC2)cc1]

### N,N-diisopentyl-8-(4-methoxyphenyl)-9-(3-(piperidin-1-yl)propyl)-9H-purine-2-carboxamide

### Intermediate 53A

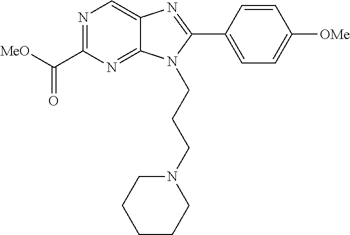 [SMILES: COC(=O)c1ncc2nc(-c3ccc(OC)cc3)n(CCCN3CCCCC3)c2n1]

### methyl 8-(4-methoxyphenyl)-9-(3-(piperidin-1-yl)propyl)-9H-purine-2-carboxylate

A mixture of methyl 6-chloro-8-(4-methoxyphenyl)-9-(3-(piperidin-1-yl)propyl)-9H-purine-2-carboxylate (9 mg, 0.02 mmol) and 5% palladium on barium carbonate (1 mg) in ethanol (2 mL) was stirred under an hydrogen atmosphere for 12 hours at room temperature. The mixture was filtered through Celite and concentrated to afford the title compound as a beige solid. LCMS m/z 410.2 (M+H)⁺, ret. time=1.89 min.

### Intermediate 53B

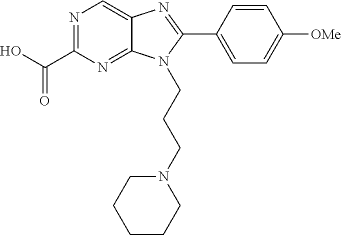 [SMILES: COc1ccc(-c2nc3cnc(C(=O)O)nc3n2CCCN2CCCCC2)cc1]

### 8-(4-methoxyphenyl)-9-(3-(piperidin-1-yl)propyl)-9H-purine-2-carboxylic acid

The title compound was prepared from methyl 8-(4-methoxyphenyl)-9-(3-(piperidin-1-yl)propyl)-9H-purine-2-carboxylate using the procedure described for the preparation of Intermediate 45E.

### Example 53

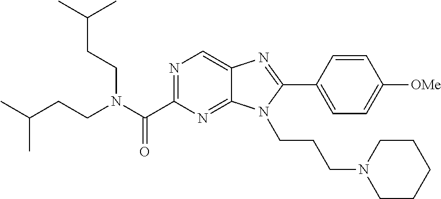 [SMILES: COc1ccc(-c2nc3cnc(C(=O)N(CCC(C)C)CCC(C)C)nc3n2CCCN2CCCCC2)cc1]

### N,N-diisopentyl-8-(4-methoxyphenyl)-9-(3-(piperidin-1-yl)propyl)-9H-purine-2-carboxamide

The title compound was prepared from 8-(4-methoxyphenyl)-9-(3-(piperidin-1-yl)propyl)-9H-purine-2-carboxylic acid and diisopentylamine according to the procedure described for the preparation of Example 2. The reaction was carried out at 65° C. LCMS m/z 535.4 (M+H)⁺, ret. time=2.45 min.

### Example 60

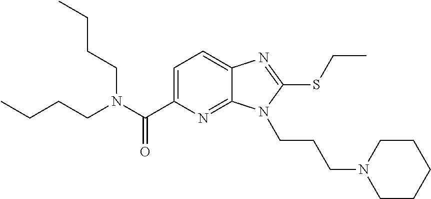 [SMILES: CCCCN(CCCC)C(=O)c1ccc2nc(SCC)n(CCCN3CCCCC3)c2n1]

### N,N-dibutyl-2-(ethylthio)-3-(3-(piperidin-1-yl)propyl)-3H-imidazo[4,5-b]pyridine-5-carboxamide

### Intermediate 60A

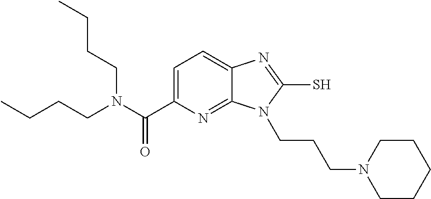 [SMILES: CCCCN(CCCC)C(=O)c1ccc2nc(S)n(CCCN3CCCCC3)c2n1]

### N,N-dibutyl-2-mercapto-3-(3-(piperidin-1-yl)propyl)-3H-imidazo[4,5-b]pyridine-5-carboxamide

(See Org. Synth. Coll. Vol. 4, p. 569 (1963)). A mixture of Intermediate 3D i.e. 5-amino-N,N-dibutyl-6-((3-(piperidin-1-yl)propyl)amino)picolinamide (0.5 g, 1.2 mmol;) and potassium ethyl xanthate (0.58 g, 3.6 mmol) in ethanol (30 mL) and water (4 mL) was heated to reflux for 12 hours. Norit (120 mg) was then carefully added and the mixture was heated to reflux for 10 minutes. Norit was then removed by filtration and the filtrate was heated to 60-70° C. Water (30 mL) and 25% acetic acid in water (7.5 mL) were added and the mixture was allowed to cool to room temperature. The mixture was concentrated under reduced pressure and a 1N sodium hydroxide solution (15 mL) and ethyl acetate (20 mL) were added. The separated aqueous layer was extracted with ethyl acetate (3×10 mL) and the combined organic layers were washed with brine, dried (sodium sulphate), filtered and evaporated. The residue was purified by flash-chromatography using methanol-dichloromethane as eluant (0 to 50% over 15 minutes). LCMS m/z 432.3 (M+H)⁺, ret. time=2.19 min.

### Example 60

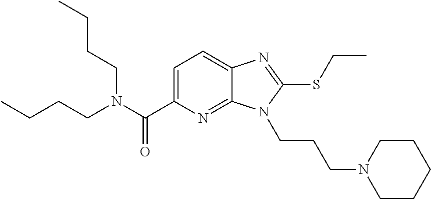 [SMILES: CCCCN(CCCC)C(=O)c1ccc2nc(SCC)n(CCCN3CCCCC3)c2n1]

### N,N-dibutyl-2-(ethylthio)-3-(3-(piperidin-1-yl)propyl)-3H-imidazo[4,5-b]pyridine-5-carboxamide

To a solution of N,N-dibutyl-2-mercapto-3-(3-(piperidin-1-yl)propyl)-3H-imidazo[4,5-b]pyridine-5-carboxamide (240 mg, 0.56 mmol) in methanol at room temperature was added a 25% solution of sodium methoxide in methanol (170 uL, 0.78 mmol) and ethyl iodide (62 uL, 0.78 mmol). The mixture was stirred at 30° C. for 12 hours and acetic acid was added. The mixture was concentrated under reduced pressure and a 10% solution of sodium bicarbonate was added to neutralize. Ethyl acetate was added and the separated aqueous layer was extracted with ethyl acetate (3×10 mL) and the combined organic layers were washed with brine, dried (sodium sulphate), filtered and evaporated. The residue was purified by flash-chromatography using methanol-dichloromethane as eluant (0 to 20% over 10 minutes). LCMS m/z 460.3 (M+H)⁺, ret. time=2.22 min.

### Example 61

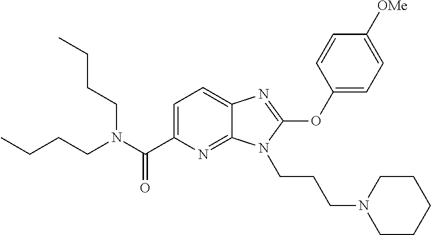 [SMILES: CCCCN(CCCC)C(=O)c1ccc2nc(Oc3ccc(OC)cc3)n(CCCN3CCCCC3)c2n1]

### N,N-dibutyl-2-(4-methoxyphenoxy)-3-(3-(piperidin-1-yl)propyl)-3H-imidazo[4,5-b]pyridine-5-carboxamide

### Intermediate 61A

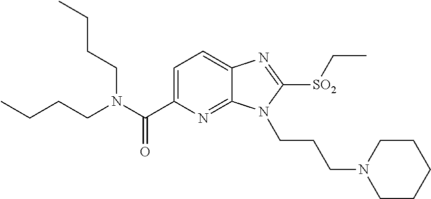 [SMILES: CCCCN(CCCC)C(=O)c1ccc2nc(S(=O)(=O)CC)n(CCCN3CCCCC3)c2n1]

### N,N-dibutyl-2-(ethylsulfonyl)-3-(3-(piperidin-1-yl)propyl)-3H-imidazo[4,5-b]pyridine-5-carboxamide

A solution of potassium permanganate (94 mg, 0.6 mmol) in water (3 mL) was added dropwise to a solution of Example 68 i.e. N,N-dibutyl-2-(ethylthio)-3-(3-(piperidin-1-yl)propyl)-3H-imidazo[4,5-b]pyridine-5-carboxamide (0.16 g, 0.35 mmol) in acetic acid (2.0 mL) at room temperature. The mixture was stirred at room temperature for 2 hours and a 1N solution of sodium hydroxide was added to neutralize. The resulting mixture was filtered and the filtrate was extracted with dichloromethane (3×15 mL). The combined organic layers were washed with brine, dried (sodium sulphate), filtered and evaporated. An aliquot was purified by reverse-phase preparative HPLC (Solvent A: MeOH:H₂O:TFA (5:95:0.05). Solvent B: MeOH:H₂O:TFA (95:5:0.05). Gradient 30 to 100% B in 12 min. Column: Zorbax SB-C18 PrepHT, 5 microns, 21.2 15×100 mm. Wavelength 220 nm). LCMS m/z 492.3 (M+H)*, ret. time. 2.18 min.

### Example 61

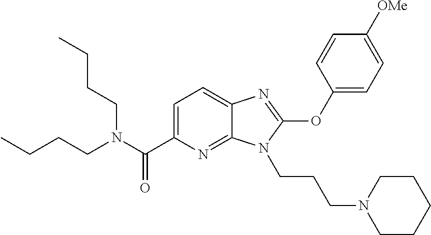 [SMILES: CCCCN(CCCC)C(=O)c1ccc2nc(Oc3ccc(OC)cc3)n(CCCN3CCCCC3)c2n1]

### N,N-dibutyl-2-(4-methoxyphenoxy)-3-(3-(piperidin-1-yl)propyl)-3H-imidazo[4,5-b]pyridine-5-carboxamide

A mixture of N,N-dibutyl-2-(ethylsulfonyl)-3-(3-(piperidin-1-yl)propyl)-3H-imidazo[4,5-b]pyridine-5-carboxamide (127 mg, 0.26 mmol), p-methoxyphenol (160 mg, 1.3 mmol) and diisopropylethylamine (225 uL, 1.3 mmol) was heated to 130° C. in a sealed tube for 12 hours. Upon cooling, volatiles were removed under reduced pressure and the residue was purified by reverse-phase preparative HPLC (Solvent A: MeOH:H₂O:TFA (5:95:0.05). Solvent B: MeOH:H₂O:TFA (95:5:0.05). Gradient 30 to 100% B in 13 min. Column: Zorbax SB-C18 PrepHT, 5 microns, 21.2×100 mm. Wavelength 220 nm). LCMS m/z 522.3 (M+H)⁺, ret. time=2.29 min

### Example 73

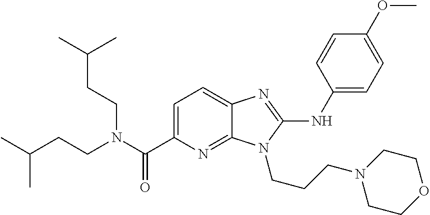 [SMILES: COc1ccc(Nc2nc3ccc(C(=O)N(CCC(C)C)CCC(C)C)nc3n2CCCN2CCOCC2)cc1]

### N,N-diisopentyl-2-((4-methoxyphenyl)amino)-3-(3-morpholinopropyl)-33H-imidazo[4,5-b]pyridine-5-carboxamide

### Intermediate 73A

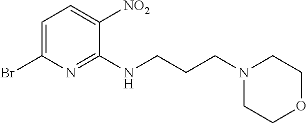 [SMILES: O=[N+]([O-])c1ccc(Br)nc1NCCCN1CCOCC1]

### 6-bromo-N-(3-morpholinopropyl)-3-nitropyridin-2-amine

Intermediate 86A was prepared according to the procedure described for the synthesis of Intermediate 1B. LCMS m/z 347.1 (M+H)⁺, ret. time=1.82 min.

### Intermediate 73B

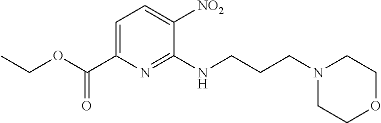 [SMILES: CCOC(=O)c1ccc([N+](=O)[O-])c(NCCCN2CCOCC2)n1]

### ethyl 6-((3-morpholinopropyl)amino)-5-nitropicolinate

A calorimetric bomb was charged with 6-bromo-N-(3-morpholinopropyl)-3-nitropyridin-2-amine (1 g, 2.90 mmol) in a mixture of MeOH (30 ml) and DMF (15.00 ml), then triethylamine (1.211 ml, 8.69 mmol), triphenylphosphine (0.036 g, 0.136 mmol) and PdOAc2 (0.033 g, 0.145 mmol) were added in this order. The mixture was heated at 60° C. under 300 psi of carbon monoxide (0.081 g, 2.90 mmol) for 16 h. The calorimetric bomb was allowed to cool to room temperature and carbon monoxide was removed in a well ventilated hood. After concentration, ethyl acetate (20 mL) and saturated sodium bicarbobante (20 mL) were added. The separated aqueous layer was extracted with ethyl acetate (3×10 mL) and the combined organic layers were washed with brine, filtered and concentrated. The residue was dissolved in dichloromethane/ethyl acetate/methanol, loaded onto silica gel and, after removal of dichloromethane/ethyl acetate/methanol under vacuum, purified by column chromatography: ISCO gold, 0% to 20% MeOH in dichloromethane over 9 min. The pooled fractions were concentrated to afford ethyl 6-((3-morpholinopropyl)amino)-5-nitropicolinate (0.66 g, 1.951 mmol, 67.3% yield) as an orange solid. LCMS m/z 339.2 (M+H)⁺, ret. time=1.95 min.

### Intermediate 73C

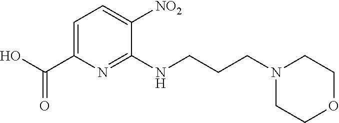 [SMILES: O=C(O)c1ccc([N+](=O)[O-])c(NCCCN2CCOCC2)n1]

### 6-((3-morpholinopropyl)amino)-5-nitropicolinic acid

A suspension of ethyl 6-((3-morpholinopropyl)amino)-5-nitropicolinate (0.66 g, 1.951 mmol) in concentrated hydrochloric acid (10 ml, 120 mmol) was stirred at 70° C. for 4 hours. The mixture was allowed to cool to room temperature and concentrated to afford 6-((3-morpholinopropyl)amino)-5-nitropicolinic acid (0.599 g, 1.931 mmol, 99% yield) as a yellow solid. LCMS m/z 311.1 (M+H)⁺, ret. time=0.60 min

### Intermediate 73D

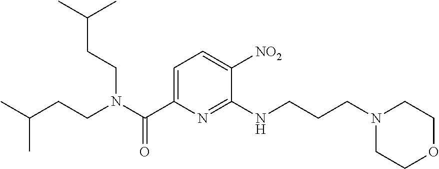 [SMILES: CC(C)CCN(CCC(C)C)C(=O)c1ccc([N+](=O)[O-])c(NCCCN2CCOCC2)n1]

### N, N-diisopentyl-6-((3-morpholinopropyl)amino)-5-nitropicolinamide

To a solution of 6-((3-morpholinopropyl)amino)-5-nitropicolinic acid, HCl (0.676 g, 1.95 mmol), EDCI (0.897 g, 4.68 mmol), HOBt (0.717 g, 4.68 mmol) and triethylamine (1.359 ml, 9.75 mmol) in DMF (10 ml) was added diisopentylamine (0.797 ml, 3.90 mmol). The mixture was stirred at 65° C. for 3 h. Ethyl acetate was added (60 mL) and the mixture was washed with water (2×20 mL), 10% sodium bicarbonate (10 mL), brine, dried over sodium sulfate, filtered and concentrated. The residue was purified by flash chromatography: ISCO Gold 40 g eluting with 0% to 10% MeOH in DCM over 15 min to afford N,N-diisopentyl-6-((3-morpholinopropyl)amino)-5-nitropicolinamide (0.49 g, 1.090 mmol, 56% yield) as a yellow solid. LCMS m/z 450.3 (M+H)⁺, ret. time=2.23 min

### Intermediate 73E

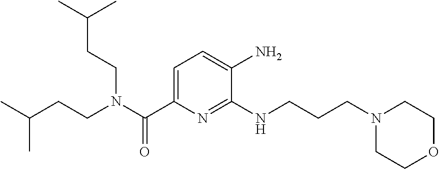 [SMILES: CC(C)CCN(CCC(C)C)C(=O)c1ccc(N)c(NCCCN2CCOCC2)n1]

### 5-amino-N,N-diisopentyl-6-((3-morpholinopropyl)amino)picolinamide

To a stirred solution of N,N-diisopentyl-6-((3-morpholinopropyl)amino)-5-nitropicolinamide (0.6 g, 1.335 mmol) in 37% solution of hydrochloric acid in water (2.67 ml) at 0° C. was added portionwise tin dichloride dihydrate (1.304 g, 5.61 mmol). The resulting suspension was transferred to a preheated oil bath at 90° C. and stirred 10 min. The mixture was cooled in an ice bath and adjusted to pH=10 with 15% NaOH aq. solution. The mixture was stirred for 15 min. Celite and dichloromethane (150 ml) were added and the mixture was filtered on Celite and washed with dichloromethane (100 ml). The filtrate was separated and extracted with dichloromethane (100 ml×2). The combined organics were dried over sodium sulfate, filtered and concentrated to afford 5-amino-N,N-diisopentyl-6-((3-morpholinopropyl)amino)picolinamide (0.55 g, 0.524 mmol, 39.3% yield) as a purple oil. LCMS m/z 420.3 (M+H)⁺, ret. time=2.19 min

### Example 73

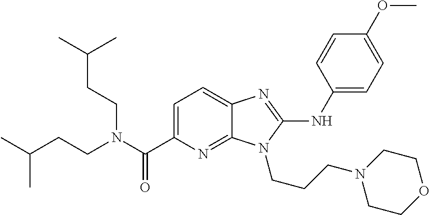 [SMILES: COc1ccc(Nc2nc3ccc(C(=O)N(CCC(C)C)CCC(C)C)nc3n2CCCN2CCOCC2)cc1]

### N,N-diisopentyl-2-((4-methoxyphenyl)amino)-3-(3-morpholinopropyl)-3H-imidazo[4,5-b]pyridine-5-carboxamide

To a solution of 5-amino-N,N-diisopentyl-6-((3-morpholinopropyl)amino)picolinamide (0.1 g, 0.238 mmol) and 1-isothiocyanato-4-methoxybenzene (0.050 ml, 0.357 mmol) in THF (2 ml) was added dicyclohexylcarbodiimide (0.108 g, 0.524 mmol) at room temperature and the mixture was stirred at 65° C. for 12 hours. The mixture was allowed to cool and treated with 1N HCl (2 ml) solution. Ethyl acetate (2 ml) was added and the resulting mixture was stirred for 30 min. The precipitate was filtered off and the filtrate was separated in two layers. The organic layer was further washed with 1N HCl (2 ml). Concentrated ammonium hydroxide was added to the combined aqueous layers which were extracted with ethyl acetate (3×10 mL). The combined organics were dried over Na2SO4, filtered and concentrated. The residue was purified by prep HPLC: 3 injections. 20% to 100% MeOH in water-TFA over 13 min. The pooled fractions were concentrated to afford N,N-diisopentyl-2-((4-methoxyphenyl)amino)-3-(3-morpholinopropyl)-3H-imidazo[4,5-b]pyridine-5-carboxamide, 2TFA (62 mg, 0.079 mmol, 33.1% yield). LCMS m/z 551.4 (M+H)⁺, ret. time=2.29 min

The preparation of all other examples included in the Table that not specifically described herein follows one of the experimental procedures above.

The general procedures described above were used to prepare the examples described below. Reported HPLC retention time are for reverse-phase HPLC (Agilent, 1200 series) using the conditions reported above (See “General” section). Mass spectra were recorded on a 6210 G1969A LC/MSD TOF spectrometer from Agilent Technologies using the LC conditions reported above (See “General” section). IC₅₀ values were obtained using the cAMP assay described below. The letter coding used for the IC₅₀s in the Table Is as follows: A<0.1 μM; 0.1 μM<B<1 μM; 1 μM<C<15 μM.

TABLE

| Cpd | | HPLC | MS: | IC50 |
|---|---|---|---|---|
| No | Structure | RT (min) | (M + H)⁺ | (nM) |
| 1 | 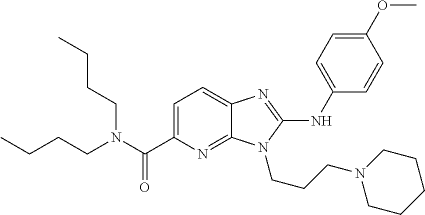 [SMILES: CCCCN(CCCC)C(=O)c1ccc2nc(Nc3ccc(OC)cc3)n(CCCN3CCCCC3)c2n1] | 1.78 | 521.4 | A |
| 2 | 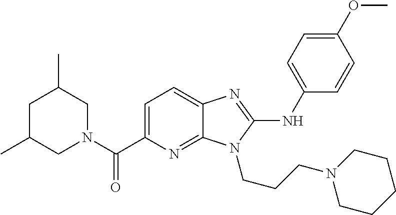 [SMILES: COc1ccc(Nc2nc3ccc(C(=O)N4CC(C)CC(C)C4)nc3n2CCCN2CCCCC2)cc1] | 1.65 | 505.4 | C |
| 3 | 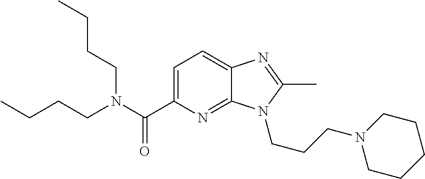 [SMILES: CCCCN(CCCC)C(=O)c1ccc2nc(C)n(CCCN3CCCCC3)c2n1] | 1.72 | 414.3 | C |
| 4 | 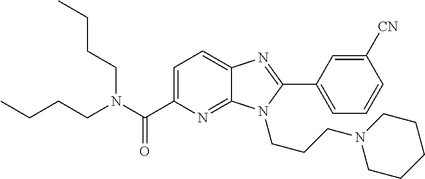 [SMILES: CCCCN(CCCC)C(=O)c1ccc2nc(-c3cccc(C#N)c3)n(CCCN3CCCCC3)c2n1] | 1.88 | 501.4 | C |
| 5 | 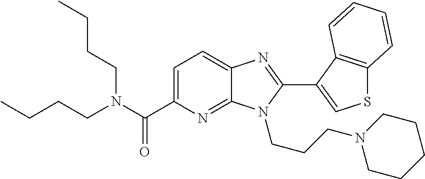 [SMILES: CCCCN(CCCC)C(=O)c1ccc2nc(-c3csc4ccccc34)n(CCCN3CCCCC3)c2n1] | 2.01 | 532.3 | B |
| 7 | 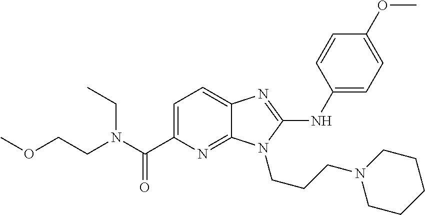 [SMILES: CCN(CCOC)C(=O)c1ccc2nc(Nc3ccc(OC)cc3)n(CCCN3CCCCC3)c2n1] | 1.51 | 495.3 | C |
| 8 | 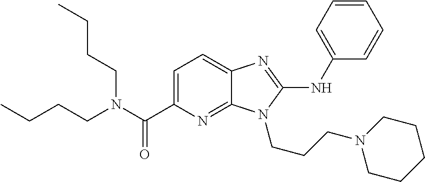 [SMILES: CCCCN(CCCC)C(=O)c1ccc2nc(Nc3ccccc3)n(CCCN3CCCCC3)c2n1] | 1.87 | 491.4 | A |
| 9 | 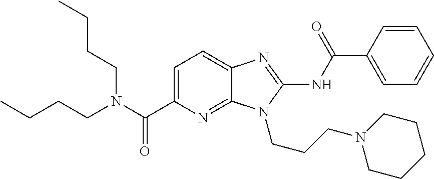 [SMILES: CCCCN(CCCC)C(=O)c1ccc2nc(NC(=O)c3ccccc3)n(CCCN3CCCCC3)c2n1] | 2.00 | 519.4 | C |
| 10 | 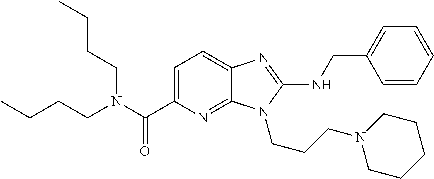 [SMILES: CCCCN(CCCC)C(=O)c1ccc2nc(NCc3ccccc3)n(CCCN3CCCCC3)c2n1] | 1.92 | 505.4 | B |
| 11 | 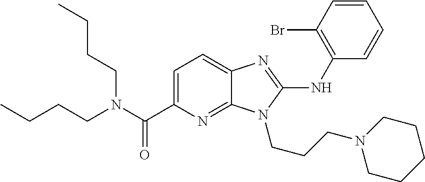 [SMILES: CCCCN(CCCC)C(=O)c1ccc2nc(Nc3ccccc3Br)n(CCCN3CCCCC3)c2n1] | 1.85 | 569.3; 571.3 | C |
| 12 | 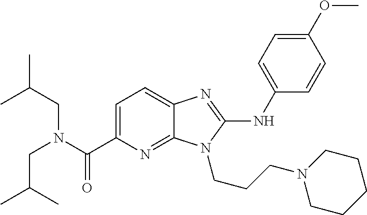 [SMILES: COc1ccc(Nc2nc3ccc(C(=O)N(CC(C)C)CC(C)C)nc3n2CCCN2CCCCC2)cc1] | 1.75 | 521.4 | B |
| 13 | 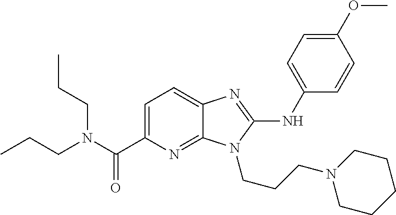 [SMILES: CCCN(CCC)C(=O)c1ccc2nc(Nc3ccc(OC)cc3)n(CCCN3CCCCC3)c2n1] | 1.57 | 493.3 | B |
| 14 | 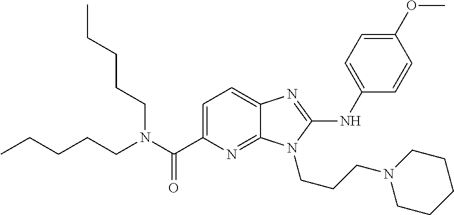 [SMILES: CCCCCN(CCCCC)C(=O)c1ccc2nc(Nc3ccc(OC)cc3)n(CCCN3CCCCC3)c2n1] | 1.82 | 549.4 | A |
| 15 | 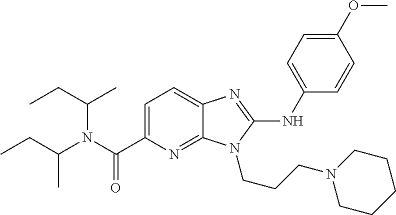 [SMILES: CCC(C)N(C(=O)c1ccc2nc(Nc3ccc(OC)cc3)n(CCCN3CCCCC3)c2n1)C(C)CC] | 1.75 | 521.4 | B |
| 16 | 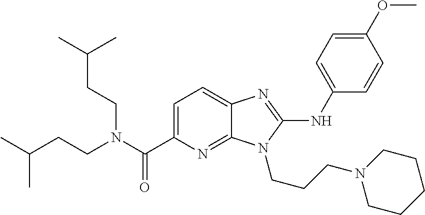 [SMILES: COc1ccc(Nc2nc3ccc(C(=O)N(CCC(C)C)CCC(C)C)nc3n2CCCN2CCCCC2)cc1] | 1.95 | 549.4 | A |
| 17 | 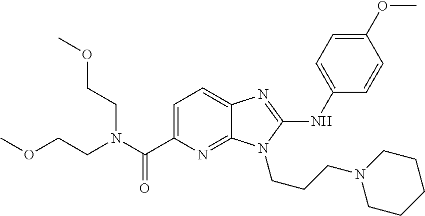 [SMILES: COCCN(CCOC)C(=O)c1ccc2nc(Nc3ccc(OC)cc3)n(CCCN3CCCCC3)c2n1] | 1.32 | 525.3 | C |
| 18 | 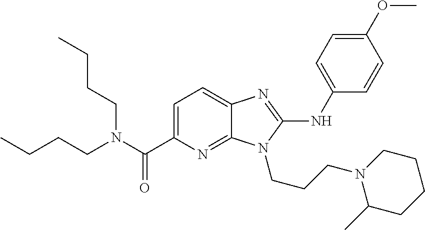 [SMILES: CCCCN(CCCC)C(=O)c1ccc2nc(Nc3ccc(OC)cc3)n(CCCN3CCCCC3C)c2n1] | 1.82 | 535.4 | A |
| 19 | 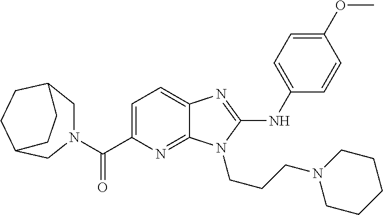 [SMILES: COc1ccc(Nc2nc3ccc(C(=O)N4CC5CCC(CC5)C4)nc3n2CCCN2CCCCC2)cc1] | 1.60 | 517.3 | C |
| 20 | 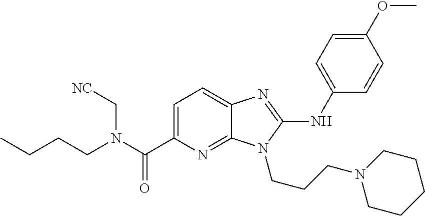 [SMILES: CCCCN(CC#N)C(=O)c1ccc2nc(Nc3ccc(OC)cc3)n(CCCN3CCCCC3)c2n1] | 1.49 | 504.3 | C |
| 21 | 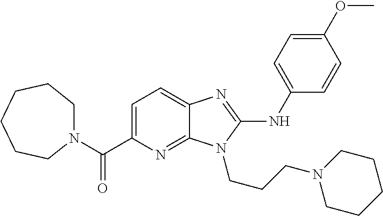 [SMILES: COc1ccc(Nc2nc3ccc(C(=O)N4CCCCCC4)nc3n2CCCN2CCCCC2)cc1] | 1.48 | 491.3 | C |
| 22 | 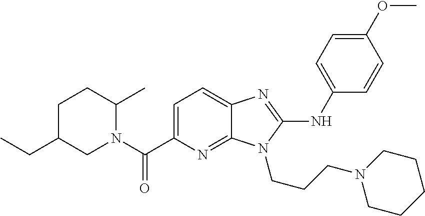 [SMILES: CCC1CCC(C)N(C(=O)c2ccc3nc(Nc4ccc(OC)cc4)n(CCCN4CCCCC4)c3n2)C1] | 1.72 | 519.4 | C |
| 23 | 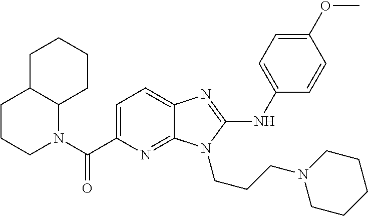 [SMILES: COc1ccc(Nc2nc3ccc(C(=O)N4CCCC5CCCCC54)nc3n2CCCN2CCCCC2)cc1] | 1.72 | 531.4 | C |
| 24 | 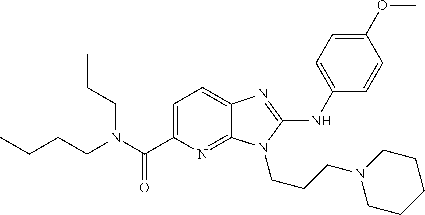 [SMILES: CCCCN(CCC)C(=O)c1ccc2nc(Nc3ccc(OC)cc3)n(CCCN3CCCCC3)c2n1] | 1.69 | 507.4 | B |
| 25 | 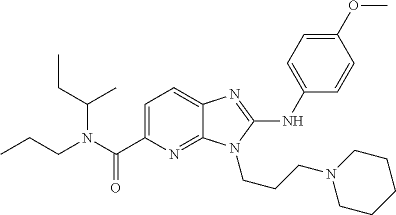 [SMILES: CCCN(C(=O)c1ccc2nc(Nc3ccc(OC)cc3)n(CCCN3CCCCC3)c2n1)C(C)CC] | 1.66 | 507.4 | B |
| 26 | 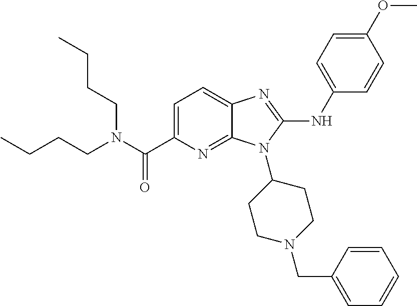 [SMILES: CCCCN(CCCC)C(=O)c1ccc2nc(Nc3ccc(OC)cc3)n(C3CCN(Cc4ccccc4)CC3)c2n1] | 1.88 | 569.4 | C |
| 27 | 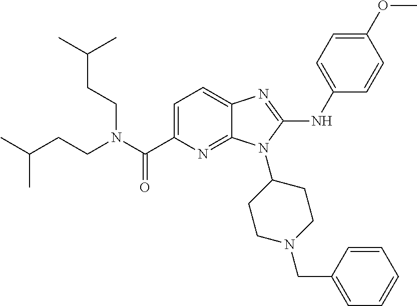 [SMILES: COc1ccc(Nc2nc3ccc(C(=O)N(CCC(C)C)CCC(C)C)nc3n2C2CCN(Cc3ccccc3)CC2)cc1] | 1.93 | 597.4 | C |
| 28 | 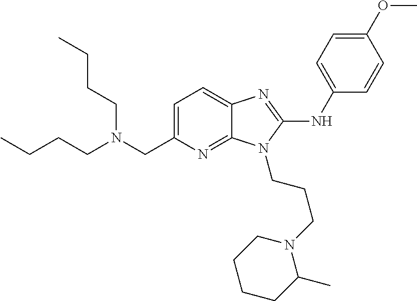 [SMILES: CCCCN(CCCC)Cc1ccc2nc(Nc3ccc(OC)cc3)n(CCCN3CCCCC3C)c2n1] | 1.40 | 521.4 | C |
| 29 | 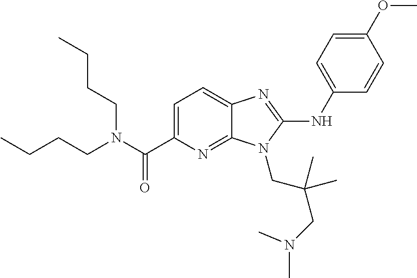 [SMILES: CCCCN(CCCC)C(=O)c1ccc2nc(Nc3ccc(OC)cc3)n(CC(C)(C)CN(C)C)c2n1] | 1.78 | 509.4 | C |
| 30 | 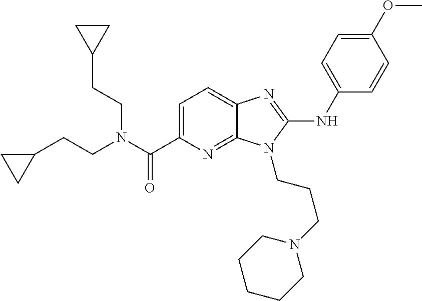 [SMILES: COc1ccc(Nc2nc3ccc(C(=O)N(CCC4CC4)CCC4CC4)nc3n2CCCN2CCCCC2)cc1] | 1.81 | 545.4 | A |
| 31 | 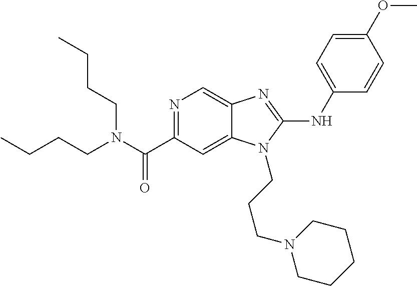 [SMILES: CCCCN(CCCC)C(=O)c1cc2c(cn1)nc(Nc1ccc(OC)cc1)n2CCCN1CCCCC1] | 1.79 | 521.4 | B |
| 32 | 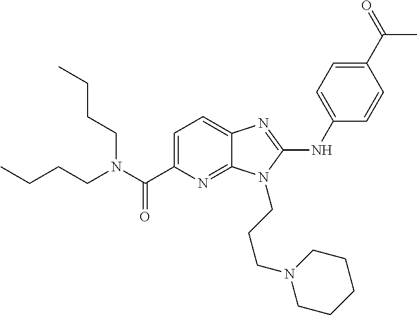 [SMILES: CCCCN(CCCC)C(=O)c1ccc2nc(Nc3ccc(C(C)=O)cc3)n(CCCN3CCCCC3)c2n1] | 1.96 | 533.4 | A |
| 33 | 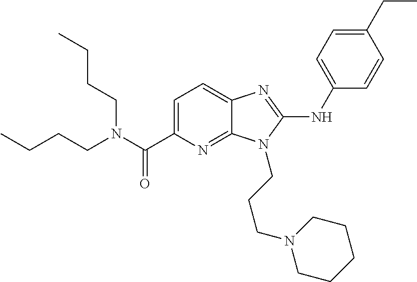 [SMILES: CCCCN(CCCC)C(=O)c1ccc2nc(Nc3ccc(CC)cc3)n(CCCN3CCCCC3)c2n1] | 1.96 | 519.4 | B |
| 34 | 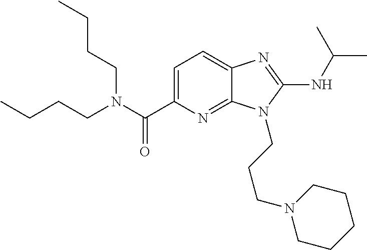 [SMILES: CCCCN(CCCC)C(=O)c1ccc2nc(NC(C)C)n(CCCN3CCCCC3)c2n1] | 1.99 | 457.4 | C |
| 35 | 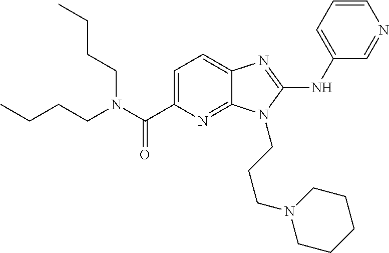 [SMILES: CCCCN(CCCC)C(=O)c1ccc2nc(Nc3cccnc3)n(CCCN3CCCCC3)c2n1] | 1.97 | 492.3 | A |
| 36 | 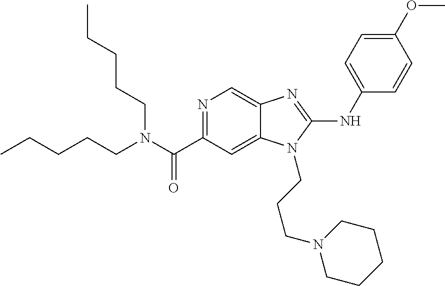 [SMILES: CCCCCN(CCCCC)C(=O)c1cc2c(cn1)nc(Nc1ccc(OC)cc1)n2CCCN1CCCCC1] | 1.99 | 549.4 | A |
| 37 | 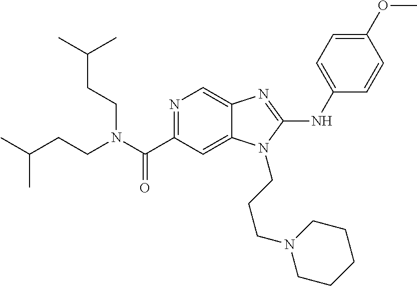 [SMILES: COc1ccc(Nc2nc3cnc(C(=O)N(CCC(C)C)CCC(C)C)cc3n2CCCN2CCCCC2)cc1] | 1.95 | 549.4 | A |
| 38 | 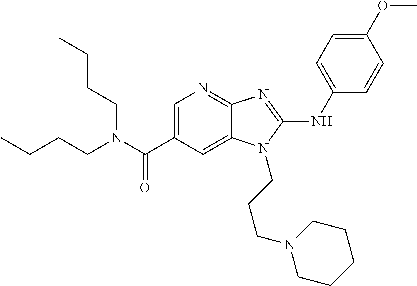 [SMILES: CCCCN(CCCC)C(=O)c1cnc2nc(Nc3ccc(OC)cc3)n(CCCN3CCCCC3)c2c1] | 1.77 | 521.4 | C |
| 39 | 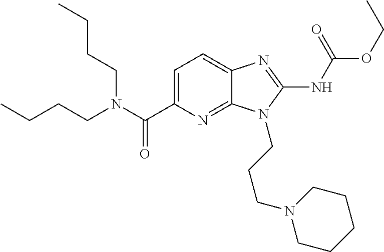 [SMILES: CCCCN(CCCC)C(=O)c1ccc2nc(NC(=O)OCC)n(CCCN3CCCCC3)c2n1] | 1.94 | 487.3 | C |
| 40 | 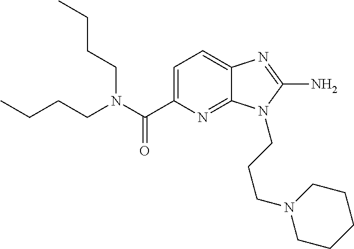 [SMILES: CCCCN(CCCC)C(=O)c1ccc2nc(N)n(CCCN3CCCCC3)c2n1] | 1.63 | 415.3 | C |
| 41 | 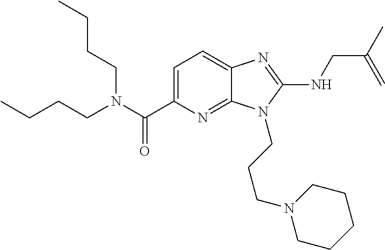 [SMILES: C=C(C)CNc1nc2ccc(C(=O)N(CCCC)CCCC)nc2n1CCCN1CCCCC1] | 1.78 | 469.4 | C |
| 42 | 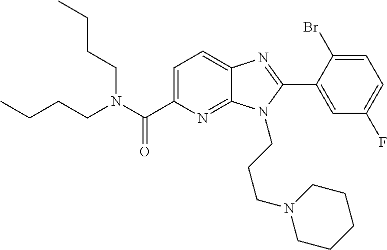 [SMILES: CCCCN(CCCC)C(=O)c1ccc2nc(-c3cc(F)ccc3Br)n(CCCN3CCCCC3)c2n1] | 1.94 | 572.2; 574.2 | C |
| 43 | 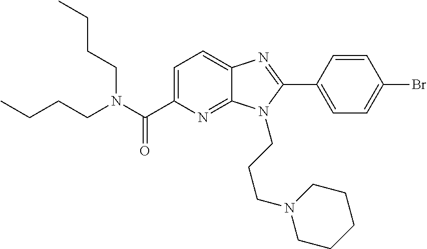 [SMILES: CCCCN(CCCC)C(=O)c1ccc2nc(-c3ccc(Br)cc3)n(CCCN3CCCCC3)c2n1] | 1.99 | 554.3; 556.3 | C |
| 44 | 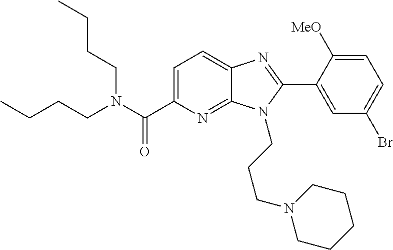 [SMILES: CCCCN(CCCC)C(=O)c1ccc2nc(-c3cc(Br)ccc3OC)n(CCCN3CCCCC3)c2n1] | 2.00 | 586.3 | B |
| 45 | 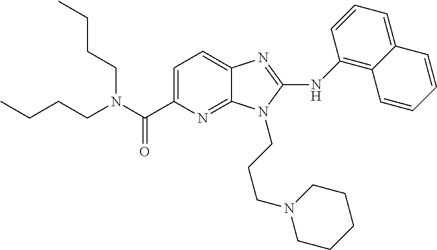 [SMILES: CCCCN(CCCC)C(=O)c1ccc2nc(Nc3cccc4ccccc34)n(CCCN3CCCCC3)c2n1] | 1.87 | 541.4 | B |
| 46 | 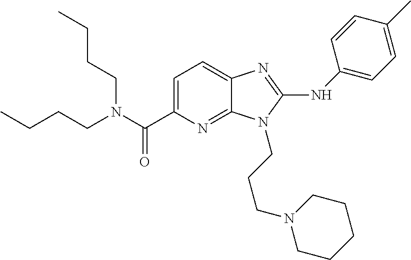 [SMILES: CCCCN(CCCC)C(=O)c1ccc2nc(Nc3ccc(C)cc3)n(CCCN3CCCCC3)c2n1] | 1.89 | 505.4 | A |
| 47 | 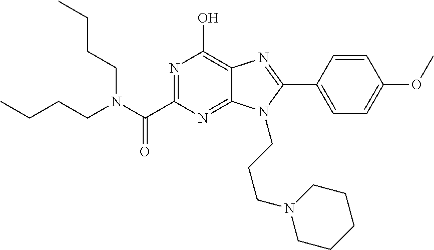 [SMILES: CCCCN(CCCC)C(=O)c1nc(O)c2nc(-c3ccc(OC)cc3)n(CCCN3CCCCC3)c2n1] | 1.77 | 523.3 | B |
| 48 | 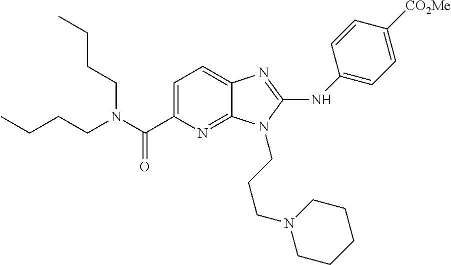 [SMILES: CCCCN(CCCC)C(=O)c1ccc2nc(Nc3ccc(C(=O)OC)cc3)n(CCCN3CCCCC3)c2n1] | 2.03 | 549.4 | A |
| 49 | 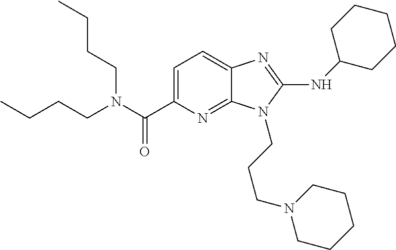 [SMILES: CCCCN(CCCC)C(=O)c1ccc2nc(NC3CCCCC3)n(CCCN3CCCCC3)c2n1] | 1.78 | 497.4 | B |
| 50 | 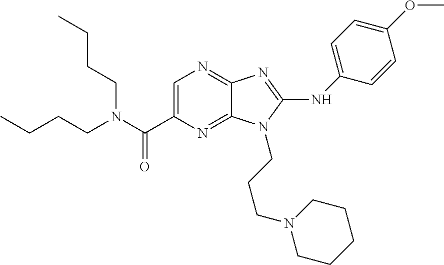 [SMILES: CCCCN(CCCC)C(=O)c1cnc2nc(Nc3ccc(OC)cc3)n(CCCN3CCCCC3)c2n1] | 1.95 | 522.4 | B |
| 51 | 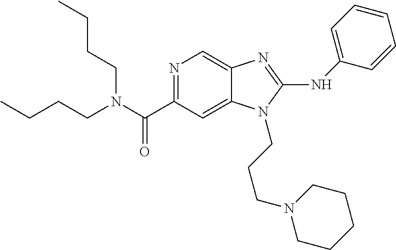 [SMILES: CCCCN(CCCC)C(=O)c1cc2c(cn1)nc(Nc1ccccc1)n2CCCN1CCCCC1] | 1.87 | 491.4 | B |
| 52 | 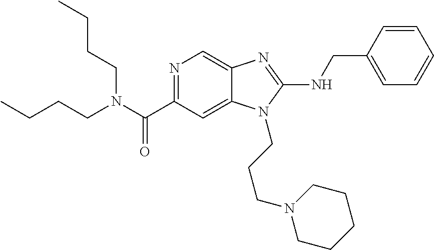 [SMILES: CCCCN(CCCC)C(=O)c1cc2c(cn1)nc(NCc1ccccc1)n2CCCN1CCCCC1] | 1.87 | 505.4 | A |
| 53 | 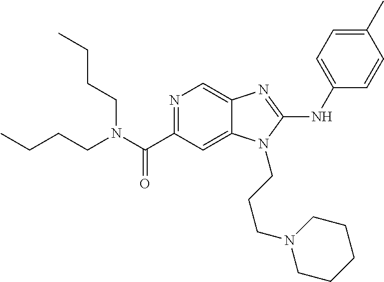 [SMILES: CCCCN(CCCC)C(=O)c1cc2c(cn1)nc(Nc1ccc(C)cc1)n2CCCN1CCCCC1] | 2.16 | 505.4 | B |
| 54 | 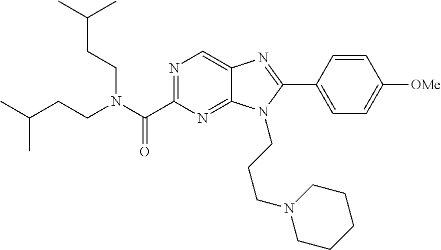 [SMILES: COc1ccc(-c2nc3cnc(C(=O)N(CCC(C)C)CCC(C)C)nc3n2CCCN2CCCCC2)cc1] | 2.09 | 535.4 | B |
| 55 | 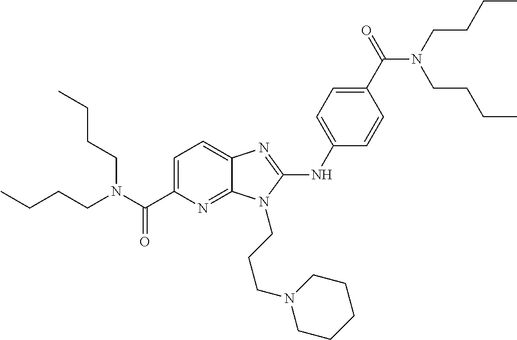 [SMILES: CCCCN(CCCC)C(=O)c1ccc(Nc2nc3ccc(C(=O)N(CCCC)CCCC)nc3n2CCCN2CCCCC2)cc1] | 2.03 | 646.5 | B |
| 56 | 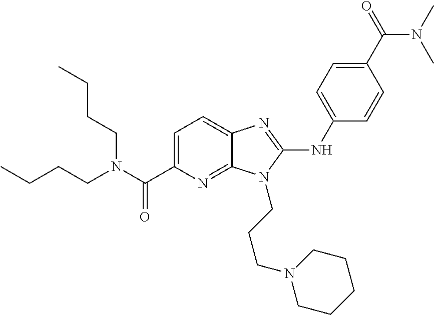 [SMILES: CCCCN(CCCC)C(=O)c1ccc2nc(Nc3ccc(C(=O)N(C)C)cc3)n(CCCN3CCCCC3)c2n1] | 1.88 | 563.4 | B |
| 57 | 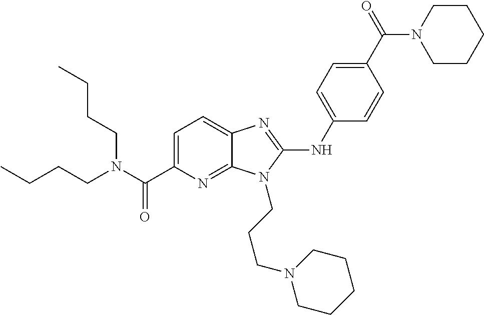 [SMILES: CCCCN(CCCC)C(=O)c1ccc2nc(Nc3ccc(C(=O)N4CCCCC4)cc3)n(CCCN3CCCCC3)c2n1] | 2.03 | 602.4 | B |
| 58 | 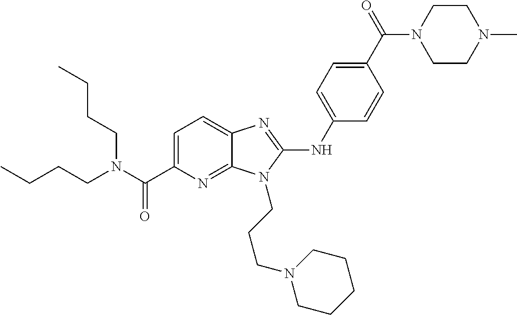 [SMILES: CCCCN(CCCC)C(=O)c1ccc2nc(Nc3ccc(C(=O)N4CCN(C)CC4)cc3)n(CCCN3CCCCC3)c2n1] | 1.71 | 617.4 | B |
| 59 | 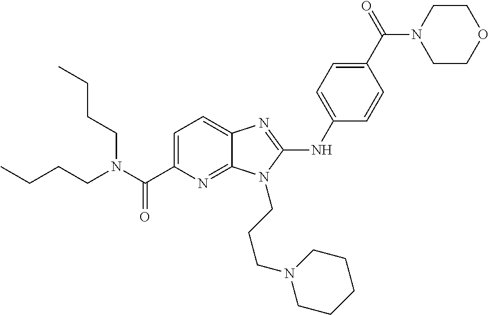 [SMILES: CCCCN(CCCC)C(=O)c1ccc2nc(Nc3ccc(C(=O)N4CCOCC4)cc3)n(CCCN3CCCCC3)c2n1] | 1.89 | 604.4 | B |
| 60 | 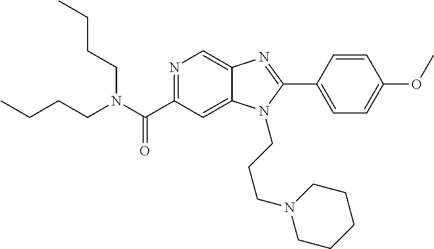 [SMILES: CCCCN(CCCC)C(=O)c1cc2c(cn1)nc(-c1ccc(OC)cc1)n2CCCN1CCCCC1] | 1.91 | 506.4 | C |
| 61 | 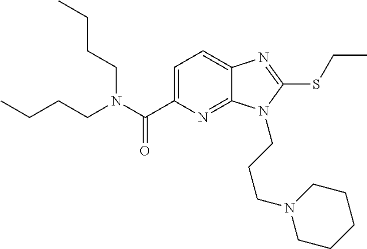 [SMILES: CCCCN(CCCC)C(=O)c1ccc2nc(SCC)n(CCCN3CCCCC3)c2n1] | 1.96 | 460.3 | C |
| 62 | 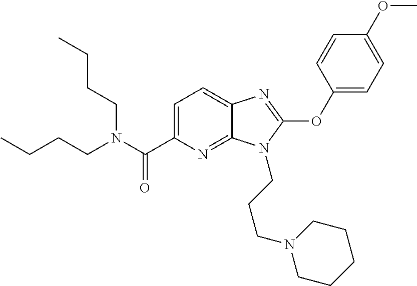 [SMILES: CCCCN(CCCC)C(=O)c1ccc2nc(Oc3ccc(OC)cc3)n(CCCN3CCCCC3)c2n1] | 1.98 | 522.4 | B |
| 63 | 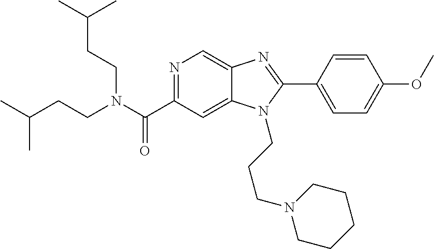 [SMILES: COc1ccc(-c2nc3cnc(C(=O)N(CCC(C)C)CCC(C)C)cc3n2CCCN2CCCCC2)cc1] | 2.05 | 534.4 | B |
| 64 | 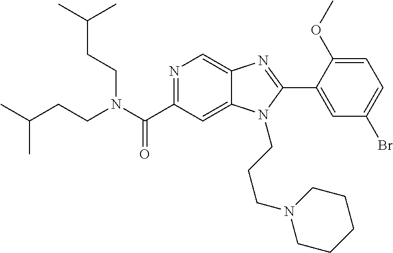 [SMILES: COc1ccc(Br)cc1-c1nc2cnc(C(=O)N(CCC(C)C)CCC(C)C)cc2n1CCCN1CCCCC1] | 2.00 | 612.3; 614.3 | C |
| 65 | 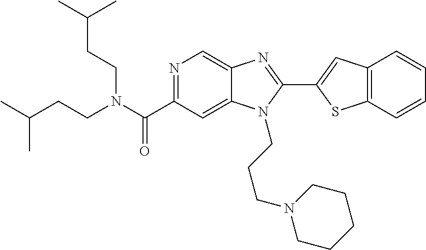 [SMILES: CC(C)CCN(CCC(C)C)C(=O)c1cc2c(cn1)nc(-c1cc3ccccc3s1)n2CCCN1CCCCC1] | 2.18 | 560.3 | A |
| 66 | 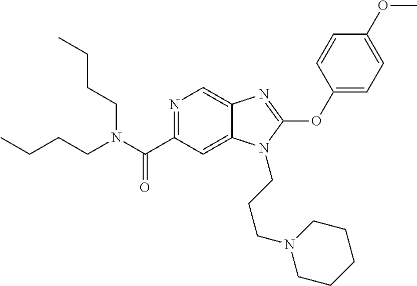 [SMILES: CCCCN(CCCC)C(=O)c1cc2c(cn1)nc(Oc1ccc(OC)cc1)n2CCCN1CCCCC1] | 1.91 | 522.3 | B |
| 67 | 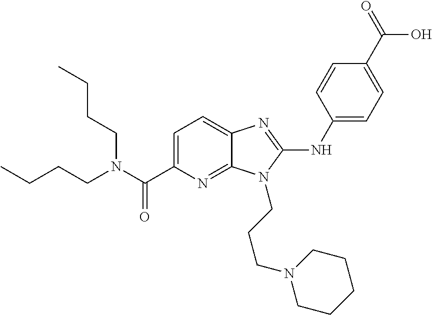 [SMILES: CCCCN(CCCC)C(=O)c1ccc2nc(Nc3ccc(C(=O)O)cc3)n(CCCN3CCCCC3)c2n1] | 1.93 | 534.3 | C |
| 68 | 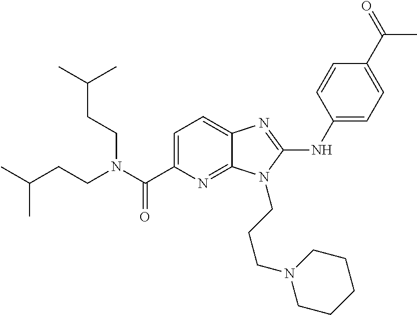 [SMILES: CC(=O)c1ccc(Nc2nc3ccc(C(=O)N(CCC(C)C)CCC(C)C)nc3n2CCCN2CCCCC2)cc1] | 2.09 | 561.4 | A |
| 69 | 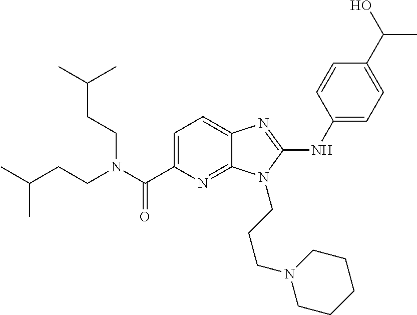 [SMILES: CC(C)CCN(CCC(C)C)C(=O)c1ccc2nc(Nc3ccc(C(C)O)cc3)n(CCCN3CCCCC3)c2n1] | 2.17 | 563.4 | A |
| 70 | 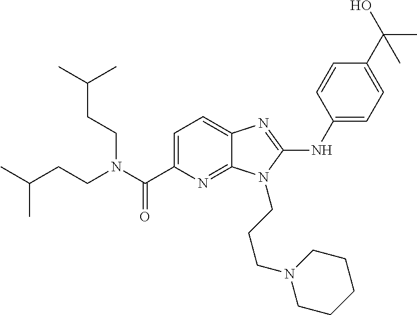 [SMILES: CC(C)CCN(CCC(C)C)C(=O)c1ccc2nc(Nc3ccc(C(C)(C)O)cc3)n(CCCN3CCCCC3)c2n1] | 2.00 | 577.4 | A |
| 71 | 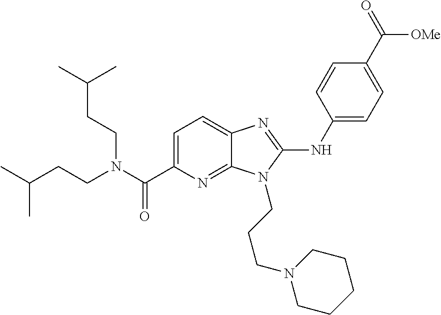 [SMILES: COC(=O)c1ccc(Nc2nc3ccc(C(=O)N(CCC(C)C)CCC(C)C)nc3n2CCCN2CCCCC2)cc1] | 2.17 | 577.4 | A |
| 72 | 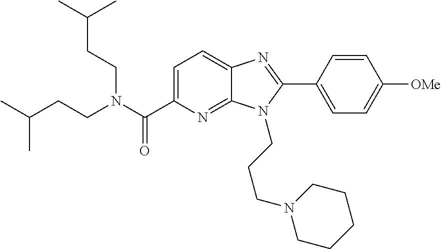 [SMILES: COc1ccc(-c2nc3ccc(C(=O)N(CCC(C)C)CCC(C)C)nc3n2CCCN2CCCCC2)cc1] | 2.08 | 534.4 | B |
| 73 | 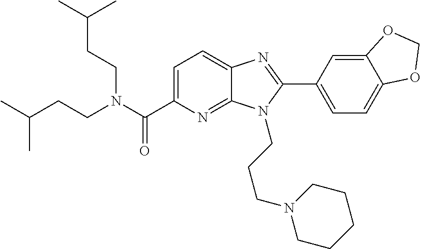 [SMILES: CC(C)CCN(CCC(C)C)C(=O)c1ccc2nc(-c3ccc4c(c3)OCO4)n(CCCN3CCCCC3)c2n1] | 2.07 | 548.4 | B |
| 74 | 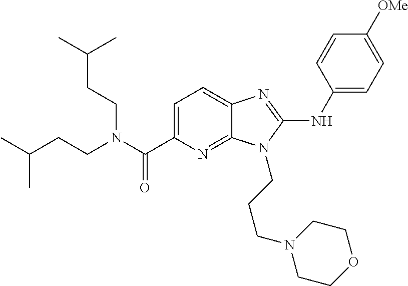 [SMILES: COc1ccc(Nc2nc3ccc(C(=O)N(CCC(C)C)CCC(C)C)nc3n2CCCN2CCOCC2)cc1] | 1.96 | 551.4 | B |
| 75 | 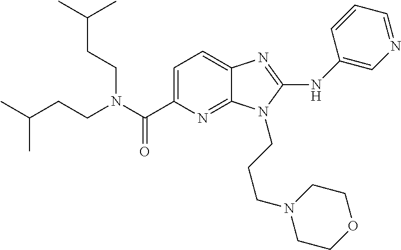 [SMILES: CC(C)CCN(CCC(C)C)C(=O)c1ccc2nc(Nc3cccnc3)n(CCCN3CCOCC3)c2n1] | 1.90 | 522.4 | B |
| 76 | 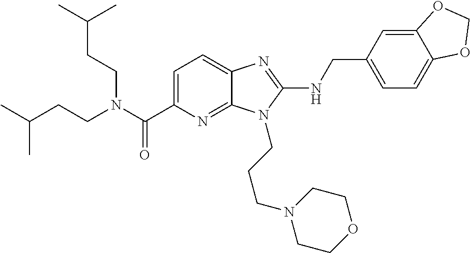 [SMILES: CC(C)CCN(CCC(C)C)C(=O)c1ccc2nc(NCc3ccc4c(c3)OCO4)n(CCCN3CCOCC3)c2n1] | 1.99 | 579.4 | B |
| 77 | 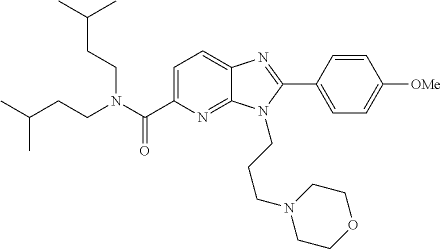 [SMILES: COc1ccc(-c2nc3ccc(C(=O)N(CCC(C)C)CCC(C)C)nc3n2CCCN2CCOCC2)cc1] | 2.06 | 536.4 | C |
| 78 | 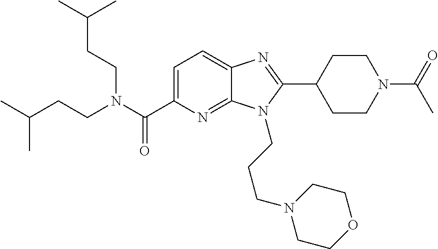 [SMILES: CC(=O)N1CCC(c2nc3ccc(C(=O)N(CCC(C)C)CCC(C)C)nc3n2CCCN2CCOCC2)CC1] | 1.93 | 555.4 | C |

IC50 value is the half maximal concentration of the tested compound to inhibit half of the maximal response of the agonist NDP-α-MSH. It is a measurement of the antagonist compound potency.

TABLE 2
The following compounds are further contemplated
Structure

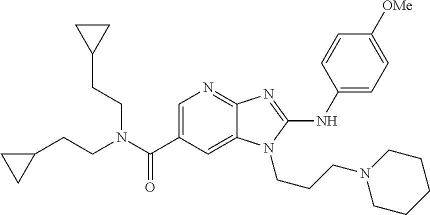 [SMILES: COc1ccc(Nc2nc3ncc(C(=O)N(CCC4CC4)CCC4CC4)cc3n2CCCN2CCCCC2)cc1]

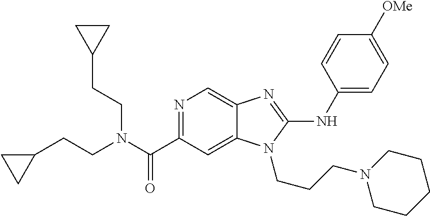 [SMILES: COc1ccc(Nc2nc3cnc(C(=O)N(CCC4CC4)CCC4CC4)cc3n2CCCN2CCCCC2)cc1]

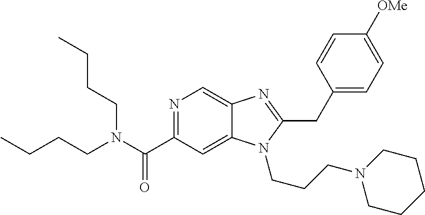 [SMILES: CCCCN(CCCC)C(=O)c1cc2c(cn1)nc(Cc1ccc(OC)cc1)n2CCCN1CCCCC1]

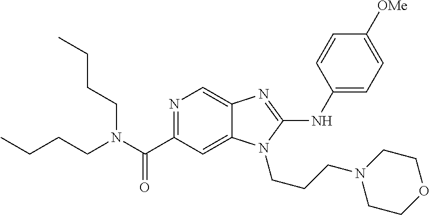 [SMILES: CCCCN(CCCC)C(=O)c1cc2c(cn1)nc(Nc1ccc(OC)cc1)n2CCCN1CCOCC1]

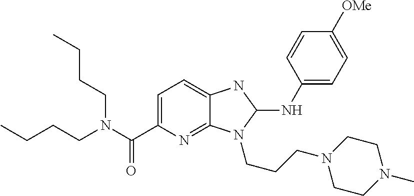 [SMILES: CCCCN(CCCC)C(=O)c1ccc2c(n1)N(CCCN1CCN(C)CC1)C(Nc1ccc(OC)cc1)N2]

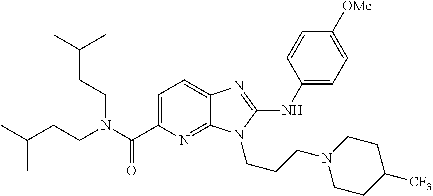 [SMILES: COc1ccc(Nc2nc3ccc(C(=O)N(CCC(C)C)CCC(C)C)nc3n2CCCN2CCC(C(F)(F)F)CC2)cc1]

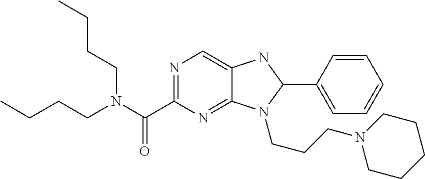 [SMILES: CCCCN(CCCC)C(=O)c1ncc2c(n1)N(CCCN1CCCCC1)C(c1ccccc1)N2]

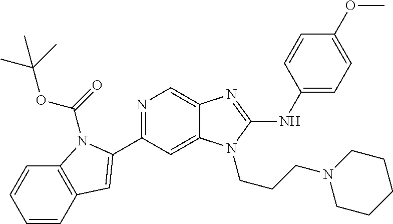 [SMILES: COc1ccc(Nc2nc3cnc(-c4cc5ccccc5n4C(=O)OC(C)(C)C)cc3n2CCCN2CCCCC2)cc1]

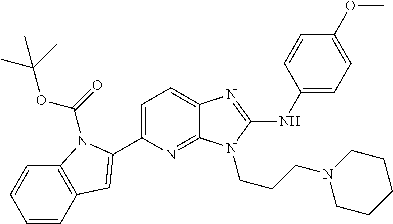 [SMILES: COc1ccc(Nc2nc3ccc(-c4cc5ccccc5n4C(=O)OC(C)(C)C)nc3n2CCCN2CCCCC2)cc1]

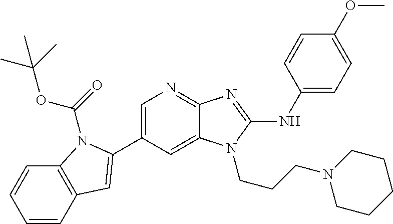 [SMILES: COc1ccc(Nc2nc3ncc(-c4cc5ccccc5n4C(=O)OC(C)(C)C)cc3n2CCCN2CCCCC2)cc1]

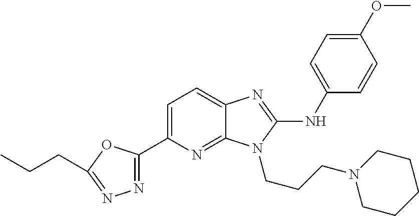 [SMILES: CCCc1nnc(-c2ccc3nc(Nc4ccc(OC)cc4)n(CCCN4CCCCC4)c3n2)o1]

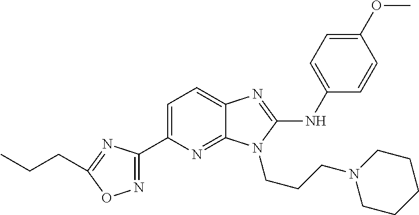 [SMILES: CCCc1nc(-c2ccc3nc(Nc4ccc(OC)cc4)n(CCCN4CCCCC4)c3n2)no1]

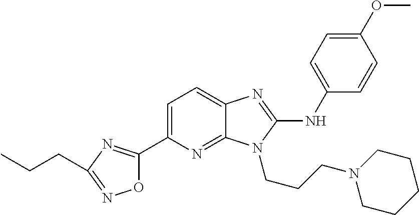 [SMILES: CCCc1noc(-c2ccc3nc(Nc4ccc(OC)cc4)n(CCCN4CCCCC4)c3n2)n1]

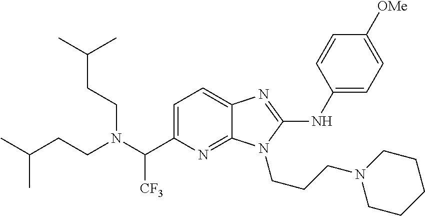 [SMILES: COc1ccc(Nc2nc3ccc(C(N(CCC(C)C)CCC(C)C)C(F)(F)F)nc3n2CCCN2CCCCC2)cc1]

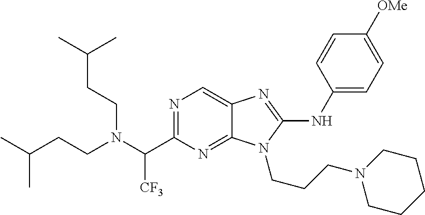 [SMILES: COc1ccc(Nc2nc3cnc(C(N(CCC(C)C)CCC(C)C)C(F)(F)F)nc3n2CCCN2CCCCC2)cc1]

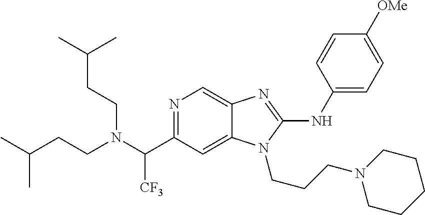 [SMILES: COc1ccc(Nc2nc3cnc(C(N(CCC(C)C)CCC(C)C)C(F)(F)F)cc3n2CCCN2CCCCC2)cc1]

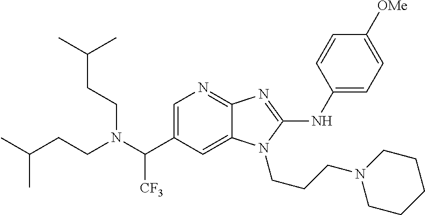 [SMILES: COc1ccc(Nc2nc3ncc(C(N(CCC(C)C)CCC(C)C)C(F)(F)F)cc3n2CCCN2CCCCC2)cc1]

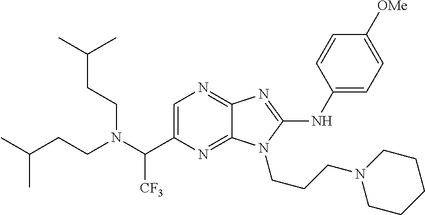 [SMILES: COc1ccc(Nc2nc3ncc(C(N(CCC(C)C)CCC(C)C)C(F)(F)F)nc3n2CCCN2CCCCC2)cc1]

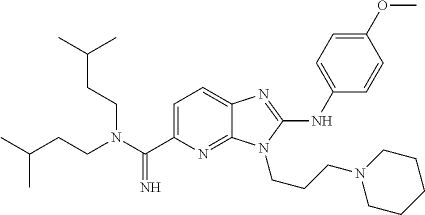 [SMILES: COc1ccc(Nc2nc3ccc(C(=N)N(CCC(C)C)CCC(C)C)nc3n2CCCN2CCCCC2)cc1]

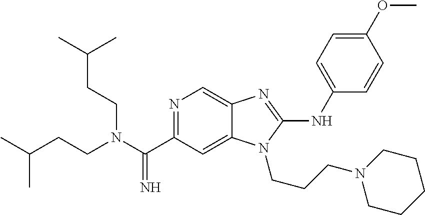 [SMILES: COc1ccc(Nc2nc3cnc(C(=N)N(CCC(C)C)CCC(C)C)cc3n2CCCN2CCCCC2)cc1]

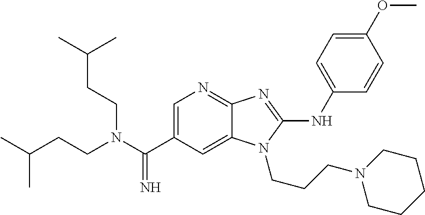 [SMILES: COc1ccc(Nc2nc3ncc(C(=N)N(CCC(C)C)CCC(C)C)cc3n2CCCN2CCCCC2)cc1]

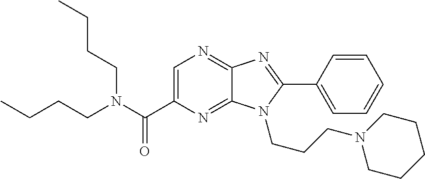 [SMILES: CCCCN(CCCC)C(=O)c1cnc2nc(-c3ccccc3)n(CCCN3CCCCC3)c2n1]

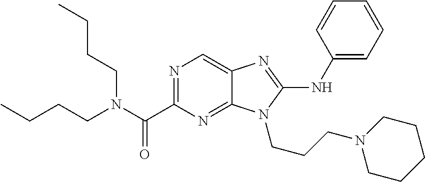 [SMILES: CCCCN(CCCC)C(=O)c1ncc2nc(Nc3ccccc3)n(CCCN3CCCCC3)c2n1]

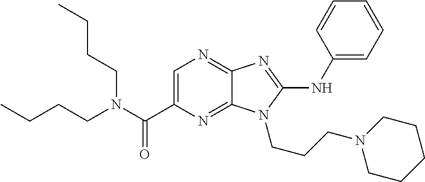 [SMILES: CCCCN(CCCC)C(=O)c1cnc2nc(Nc3ccccc3)n(CCCN3CCCCC3)c2n1]

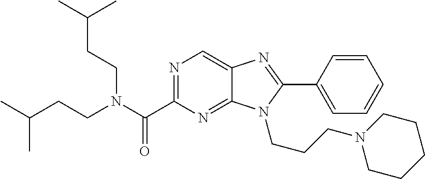 [SMILES: CC(C)CCN(CCC(C)C)C(=O)c1ncc2nc(-c3ccccc3)n(CCCN3CCCCC3)c2n1]

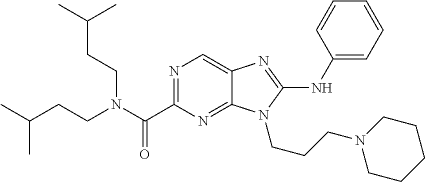 [SMILES: CC(C)CCN(CCC(C)C)C(=O)c1ncc2nc(Nc3ccccc3)n(CCCN3CCCCC3)c2n1]

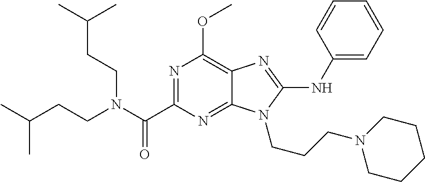 [SMILES: COc1nc(C(=O)N(CCC(C)C)CCC(C)C)nc2c1nc(Nc1ccccc1)n2CCCN1CCCCC1]

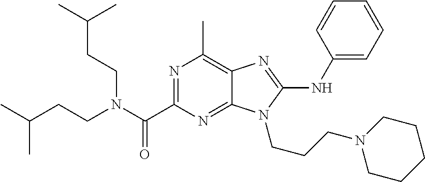 [SMILES: Cc1nc(C(=O)N(CCC(C)C)CCC(C)C)nc2c1nc(Nc1ccccc1)n2CCCN1CCCCC1]

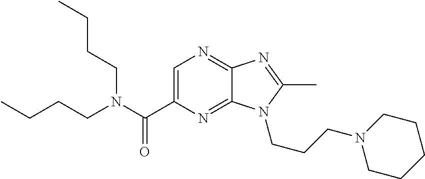 [SMILES: CCCCN(CCCC)C(=O)c1cnc2nc(C)n(CCCN3CCCCC3)c2n1]

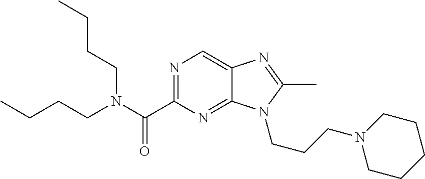 [SMILES: CCCCN(CCCC)C(=O)c1ncc2nc(C)n(CCCN3CCCCC3)c2n1]

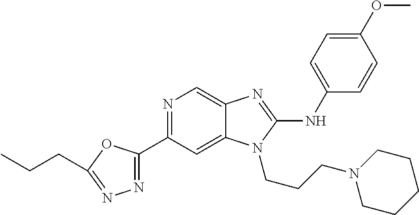 [SMILES: CCCc1nnc(-c2cc3c(cn2)nc(Nc2ccc(OC)cc2)n3CCCN2CCCCC2)o1]

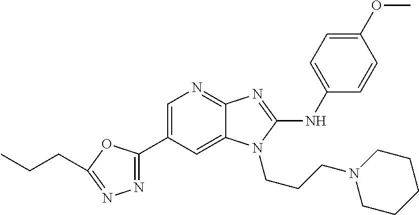 [SMILES: CCCc1nnc(-c2cnc3nc(Nc4ccc(OC)cc4)n(CCCN4CCCCC4)c3c2)o1]

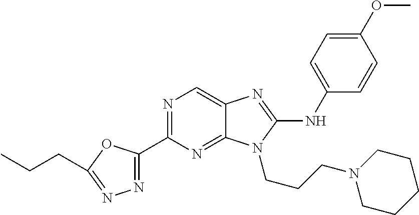 [SMILES: CCCc1nnc(-c2ncc3nc(Nc4ccc(OC)cc4)n(CCCN4CCCCC4)c3n2)o1]

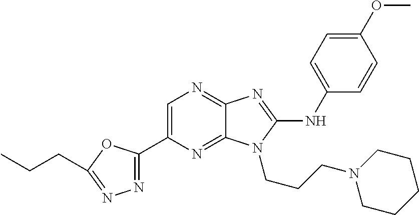 [SMILES: CCCc1nnc(-c2cnc3nc(Nc4ccc(OC)cc4)n(CCCN4CCCCC4)c3n2)o1]

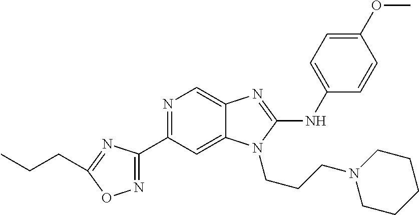 [SMILES: CCCc1nc(-c2cc3c(cn2)nc(Nc2ccc(OC)cc2)n3CCCN2CCCCC2)no1]

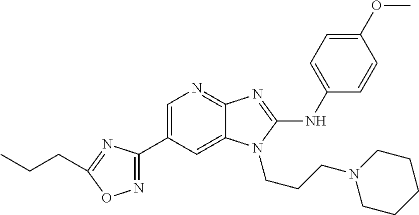 [SMILES: CCCc1nc(-c2cnc3nc(Nc4ccc(OC)cc4)n(CCCN4CCCCC4)c3c2)no1]

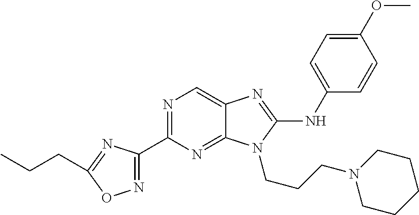 [SMILES: CCCc1nc(-c2ncc3nc(Nc4ccc(OC)cc4)n(CCCN4CCCCC4)c3n2)no1]

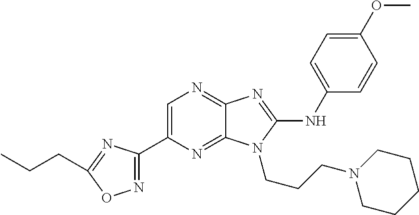 [SMILES: CCCc1nc(-c2cnc3nc(Nc4ccc(OC)cc4)n(CCCN4CCCCC4)c3n2)no1]

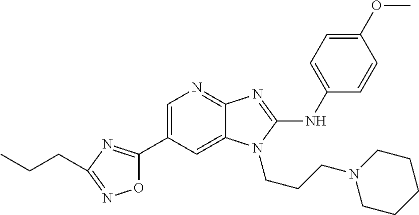 [SMILES: CCCc1noc(-c2cnc3nc(Nc4ccc(OC)cc4)n(CCCN4CCCCC4)c3c2)n1]

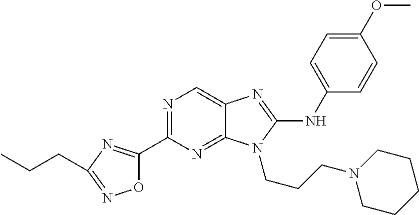 [SMILES: CCCc1noc(-c2ncc3nc(Nc4ccc(OC)cc4)n(CCCN4CCCCC4)c3n2)n1]

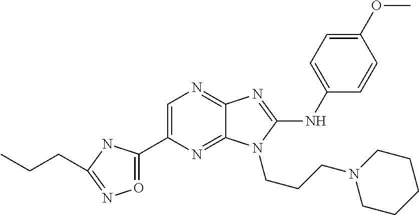

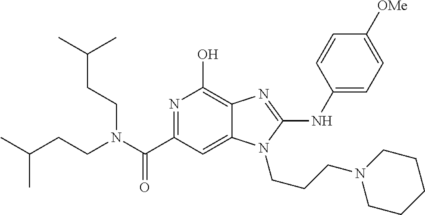 [SMILES: COc1ccc(Nc2nc3c(O)nc(C(=O)N(CCC(C)C)CCC(C)C)cc3n2CCCN2CCCCC2)cc1]

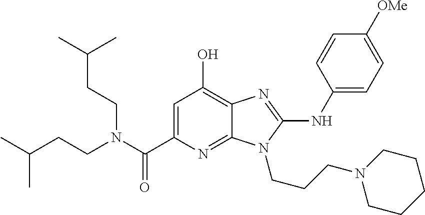 [SMILES: COc1ccc(Nc2nc3c(O)cc(C(=O)N(CCC(C)C)CCC(C)C)nc3n2CCCN2CCCCC2)cc1]

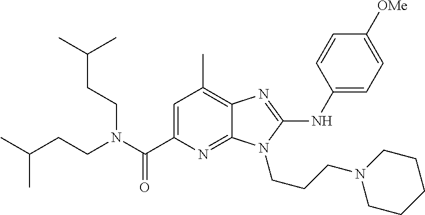 [SMILES: COc1ccc(Nc2nc3c(C)cc(C(=O)N(CCC(C)C)CCC(C)C)nc3n2CCCN2CCCCC2)cc1]

### Assays

To test the ability of a compound to modulate the stability, activity, and/or cell surface localization of an MC4R polypeptide, a labeled or unlabeled test compound is brought in contact with the MC4R protein or a fragment thereof and the amount of the test compound bound to the MC4R protein or to the fragment thereof is measured. This can be achieved for example as follows:
(a) contacting a first cell with a test compound for a time period sufficient to allow the cell to respond to said contact with the test compound;
(b) determining the conformational stability, activity, and/or cell surface localization of a MC4R polypeptide (or a fragment thereof comprising a ligand binding domain) in the cell (or on the cell surface) contacted in step (a); and comparing the stability, activity, and/or cell surface localization of the MC4R polypeptide determined in step (b) to that of an MC4R polypeptide in a control cell that has not been contacted with the test compound; wherein a detectable change in the stability, activity, and/or cell surface localization of the MC4R polypeptide in the first cell in response to contact with the test compound compared to the stability level of the MC4R polypeptide in the control cell that has not been contacted with the test compound, indicates that the test compound modulates the stability of the MC4R polypeptide and is a candidate compound for the treatment of a disorder associated with reduced MC4R stability or activity. The cell can either be a host cell transformed with a non-endogenous wild-type or mutant MC4R, or an endogenously-MC4R-expressing cell, including mutant and wild-type MC4Rs. Such cells include the “obesity neurons” such as GTI—7 cells, described above, those described in MacKenzie et al., Current Medicinal Chemistry—Immunology, Endocrine & Metabolic Agents 2004; 4: 113-117, which endogenously express MC4R, or transformed cells expressing normal or mutated, tagged MC4R such as the HEK293 cells described in Blondet et al., J Biochem 2004; 135: 541-546.

### Cyclic AMP Assay

Intracellular cyclic 3′-5′ adenosine monophosphate (cAMP) accumulation was measured using a competitive immunoassay based on HTRF (Homogeneous Time-Resolved Fluorescence) technology (cAMP dynamic-2, Cis-Bio).

HEK293T stable cell line expressing double-tagged WT-hMC4R construct were collected and washed in cAMP buffer (1XD-PBS pH 7.4, 0.1% glucose). 40,000 cells/well were then dispensed in 96-well plates in cAMP buffer [1XD-PBS, 1% BSA, 0.1% Glucose, 0.75 mM 3-isobutyl-1 methyl-xanthine (IBMX, Sigma)] and incubated either for antagonist competition assay: 1 hour at 37° C. with various concentrations of compound [0.01 nM-10 μM] followed by 30 min incubation at 37° C. with 4 nM of NDP-α-MSH (Sigma) or for agonist assay: 1 hour at 37° C. with various concentrations of compound [0.01 nM-10 μM]. 10,000 cells were transferred in 384-well plates, lysed and incubated with cAMP labeled with the dye d2 and anti-cAMP M-Antidody labeled with Cryptate following the manufacturer's protocol.

Reading of HTRF signal was performed on Artemis TR-FRET plate reader (Cosmo Bio).

### Curve Fitting

All curve fitting was conducted using non-linear regression analyses from PRISM (version 4.0c, GraphPad Inc.) and the determination of Rmax and EC50 or IC50 parameters were obtained from sigmoidal dose-response phase (variable slope) equation.

### Generation of WT-hMC4R Double Tagged Construct

A cDNA encoding a wild-type human MC4R with a 3 tandem copies of Hemaglutinine (HA) epitote sequence from UMR cDNA resource center was modified using known techniques in the art (e.g., PCR, cloning) to fused in frame at the C-terminal a venus-Yellow Fluorescent Protein (YFP) cDNA.

The resultant cDNA was then subcloned in eukaryotic expression vector, e.g. pcDNA 3.1(+) (Invitrogen).

### Generation of Double Tagged WT-hMC4R Stable Cell Line and Cell Culture

The double tagged WT-hMC4R was transfected in HEK293T cells with lipofectamine (Invitrogen) following the manufacturer's instructions and permanently transfected clonal cell lines were selected by resistance to the neomycin analog G418.

HEK293T stable cell line expressing wild-type human melanocortin 4 receptor (WT-hMC4R) containing an N-terminal 3×HA epitope tag and an intracellular C-terminal venus-yellow fluorescent protein (v-YFP) were maintained at 37° C. in humidified air containing 5% CO2 in Dulbecoo's modified Eagle's medium (DMEM) supplemented with 10% fetal bovine serum (FBS), 100 U/ml penicillin/streptomycin (DMEM complete/10% FBS). Cells were generally at 70%-80% confluence on the day of assay.

### Determining MC4R Cell Surface Expression.

Numerous assays can be used to evaluate cell surface receptor expression quantitatively to determine whether a compound can enhance the amount of MC4R trafficked to the cell surface. For example, radioactive ligand binding assays, using e.g., ¹²⁵I-NDP-αMSH, can be used to determine binding to either whole cells expressing MC4R or to cell membrane fractions. See U.S. published application 2003/0176425 for a description of one exemplary method; see also Chajlani, Peptides. 1996; 17(2):349-51. In addition, immunofluorescence staining, using either labeled antibodies or labeled MC4R (e.g., HA-tagged MC4R), may also be used. Another well-known method is fluorescence-activated cell sorting (FACS), which sorts or distinguishes populations of cells using labeled antibodies against cell surface markers, see also Lubrano-Berthelier C. et al., Human Molecular Genetics, 2003; 12(2):145-153.

It is understood that the examples and embodiments described herein are for illustrative purposes only and that various modifications or changes in light thereof will be suggested to persons skilled in the art and are to be included within the present discovery and scope of the appended claims.

1. A method stimulating the activity of a melanocortin-4 receptor (MC4R) polypeptide, comprising administering a compound to a subject or a cell, wherein said compound specifically binding to the MC4R polypeptide and stabilizing the conformational folding of said polypeptide stimulating the activity of the polypeptide, and wherein said compound is of Formula I: 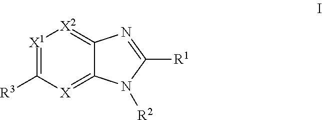
or a salt thereof,
wherein
X is N, and X¹ and X² are both CH; or X¹ is N, and X and X² are both CH; or X² is N, and X and X¹ are both CH; or X and X¹ are both N, and X² is CH or C(OH); or
X and X² are both N, and X¹ is CH;
R¹ is
1) C₁-C₆ alkyl,
2) aryl,
3) heteroaryl,
4) NHR⁴,
5) C₁-C₆alkyl-aryl,
6) C₁-C₆ alkyl-heteroaryl,
7) C(O) aryl,
8) C(O) heteroaryl,
9) OR⁴,
10) SR⁴, or
11) heterocyclyl
wherein the aryl, the heteroaryl and the heterocyclyl are optionally substituted with one or more R¹⁰ substituents;
R² is
1) C₁-C₆ alkyl-heterocyclyl,
2) C₁-C₆ alkyl-N(C₁-C₆ alkyl)₂,
3) C₁-C₆ alkyl-NHC₁-C₆ alkyl,
4) C₁-C₆ alkyl-NH₂, or
5) heterocyclyl,
wherein the heterocyclyl is optionally substituted with one or more C₁-C₆ alkyl substituents or C₁-C₆ alkyl-aryl;
R³ is
1) C(O)NR⁵R⁶,
2) CH₂NRR⁶,
3) C(O)-heterocyclyl,
4) C(NH)NR⁵R⁶, or
5) CH(CF₃)NR⁵R⁶
wherein the heterocyclyl is optionally substituted with one or more R¹⁰ substituents;
R⁴ is
1) H,
2) aryl,
3) heteroaryl,
4) C₁-C₆ alkyl,
5) C₁-C₆ alkyl-aryl,
6) C₁-C₆ alkyl-heteroaryl,
7) C₁-C₆ alkyl-NHC(O) C₁-C₆ alkyl,
8) C₁-C₆ alkyl-O— C₁-C₆ alkyl,
9) C₁-C₆ alkyl-heterocyclyl,
10) C₂-C₆ alkenyl,
11) C₃-C₇ cycloalkyl,
12) C(O)-aryl, or
13) C(O)O C₁-C₆ alkyl
wherein the aryl, the heteroaryl or the heterocyclyl is optionally substituted with one or more R¹⁰ substituents;
R⁵ and R⁶ are each independently selected from
1) C₁-C₆ alkyl,
2) C₁-C₆ alkyl-C₃-C₇ cycloalkyl,
3) C₁-C₆ alkyl-O— C₁-C₆ alkyl, or
4) C₁-C₆ alkyl-CN;
R¹⁰ is
1) CN,
2) NO₂,
3) halogen,
4) haloalkyl,
5) C₁-C₆ alkyl,
6) OH,
7) OC₁-C₆ alkyl,
8) O-aryl,
9) C(O)OH,
10) C(O)OC₁-C₆ alkyl,
11) C(O) C₁-C₆ alkyl,
12) C(O) aryl,
13) C(O) heterocyclyl,
14) C(O)NH₂,
15) C(O)NH C₁-C₆ alkyl,
16) C(O)N(C₁-C₆ alkyl)₂,
17) NH₂,
18) NHC(O) C₁-C₆ alkyl,
19) C₁-C₆ alkyl heterocyclyl,
20) C₁-C₆ alkyl (OH),
21) C₁-C₆ alkyl (NH₂),
22) N(C₁-C₆ alkyl)₂,
23) NH(C₁-C₆ alkyl),
24) aryl,
25) heteroaryl, or
26) heterocyclyl,
wherein the heterocyclyl is optionally substituted with one or more C₁-C₆ alkyl substituents.

2. The method of claim 1, wherein X is N, and X¹ and X² are both CH.

3. The method of claim 2, in which:
R¹ is
1) C₁-C₆ alkyl,
2) aryl,
3) heteroaryl,
4) NHR⁴,
5) OR⁴,
6) SR⁴, or
7) heterocyclyl,
wherein the heterocyclyl is substituted with one or more R¹⁰ substituents;
R² is
1) C₁-C₆ alkyl-heterocyclyl,
2) C₁-C₆ alkyl-N(C₁-C₆ alkyl)₂, or
3) heterocyclyl,
wherein the heterocyclyl is optionally substituted with one or more C₁-C₆ alkyl substituents or C₁-C₆ alkyl-aryl;
R³ is
1) C(O)NR⁵R⁶,
2) CH₂NRR⁶, or
3) C(O)-heterocyclyl
wherein the heterocyclyl is optionally substituted with one or more R¹⁰ substituents;
R⁴ is
1) H,
2) aryl,
3) heteroaryl,
4) C₁-C₆ alkyl,
5) C₁-C₆alkyl-aryl,
6) C(O)-aryl,
7) C₂-C₆ alkenyl,
8) C₃-C₇ cycloalkyl, or
9) C(O)O C₁-C₆ alkyl,
wherein the aryl is optionally substituted with one or more R¹⁰ substituents;
R⁵ and R⁶ are each independently selected from
1) C₁-C₆ alkyl,
2) C₁-C₆alkyl-C₃-C₇ cycloalkyl,
3) C₁-C₆ alkyl-O—C₁-C₆ alkyl, or
4) C₁-C₆ alkyl-CN; and
R¹⁰ is
1) CN,
2) halogen,
3) C₁-C₆ alkyl,
4) OC₁-C₆ alkyl,
5) C(O)OH,
6) C(O)OC₁-C₆ alkyl,
7) C(O) C₁-C₆ alkyl,
8) C(O) heterocyclyl,
9) C(O)N(C₁-C₆ alkyl)₂, or
10) C₁-C₆ alkyl (OH),
wherein the heterocyclyl is optionally substituted with one or more C₁-C₆ alkyl substituents.

4. The method of claim 3, in which
R¹ is
1) aryl,
2) heteroaryl,
3) NHR⁴, or
4) OR⁴,
wherein the aryl is optionally substituted with one R¹⁰ substituent;
R² is C₁-C₆ alkyl-heterocyclyl,
wherein the heterocyclyl is optionally substituted with one C₁-C₆ alkyl substituent;
R³ is C(O)NR⁵R⁶;
R⁴ is
1) aryl,
2) heteroaryl,
3) C₁-C₆ alkyl-aryl, or
4) C₃-C₇ cycloalkyl,
wherein the aryl is optionally substituted with one or more R¹⁰ substituents;
R⁵ and R⁶ are each independently selected from
1) C₁-C₆ alkyl,
2) C₁-C₆ alkyl-C₃-C₇ cycloalkyl; and
R¹⁰ is
1) halogen,
2) C₁-C₆ alkyl,
3) OC₁-C₆ alkyl,
4) C(O)OC₁-C₆ alkyl,
5) C(O) C₁-C₆ alkyl,
6) C(O) heterocyclyl,
7) C(O)N(C₁-C₆ alkyl)₂, or
8) C₁-C₆ alkyl (OH),
wherein the heterocyclyl is optionally substituted with one C₁-C₆ alkyl substituent.

5. The method of claim 1, wherein X¹ is N, and X and X² are both CH.

6. The method of claim 5, in which:
R¹ is
1) aryl,
2) heteroaryl,
3) NHR⁴, or
4) OR⁴,
wherein the aryl is substituted with one or more R¹⁰ substituents;
R² is C₁-C₆ alkyl-heterocyclyl;
R³ is C(O)NR₅R⁶;
R⁴ is
1) aryl, or
2) C₁-C₆ alkyl-aryl,
wherein the aryl is optionally substituted with one or more R⁰ substituents;
R⁵ and R⁶ are both C₁-C₆ alkyl; and
R¹⁰ is
1) halogen,
2) C₁-C₆ alkyl, or
3) OC₁-C₆ alkyl.

7. The method of claim 1, wherein X² is N, and X and X¹ are both CH.

8. The method of claim 7, in which:
R¹ is NHR⁴;
R² is C₁-C₆ alkyl-heterocyclyl;
R³ is C(O)NR⁵R⁶;
R⁴ is aryl substituted with one OC₁-C₆ alkyl substituent; and
R⁵ and R⁶ are both C₁-C₆ alkyl.

9. The method of claim 1, wherein X and X¹ are both N, and X² is CH.

10. The method of claim 9, in which:
R¹ is aryl substituted with one OC₁-C₆ alkyl substituent;
R² is C₁-C₆ alkyl-heterocyclyl,
R³ is C(O)NR⁵R⁶; and
R⁵ and R⁶ both C₁-C₆ alkyl.

11. The method of claim 1, wherein X and X² are both N, and X¹ is CH.

12. The method of claim 11, in which:
R¹ is NHR⁴;
R² is C₁-C₆ alkyl-heterocyclyl;
R³ is C(O)NR⁵R⁶;
R⁴ is aryl substituted with one OC₁-C₆ alkyl substituent; and
R⁵ and R⁶ are both C₁-C₆ alkyl.

13. The method of claim 1, wherein X and X¹ are both N, and X² is C(OH).

14. The method of claim 13, in which:
R¹ is aryl substituted with one OC₁-C₆ alkyl substituent;
R² is C₁-C₆ alkyl-heterocyclyl;
R³ is C(O)NR⁵R⁶; and
R⁵ and R⁶ are both C₁-C₆ alkyl.

15. The method of claim 1, the compound is selected from the group consisting of:

| Cpd | |
|---|---|
| No | Structure |
| 1 | 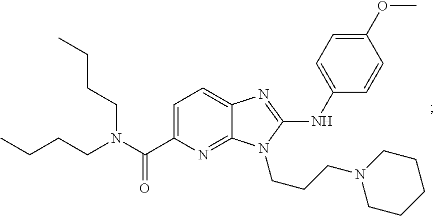 [SMILES: CCCCN(CCCC)C(=O)c1ccc2nc(Nc3ccc(OC)cc3)n(CCCN3CCCCC3)c2n1] |
| 2 | 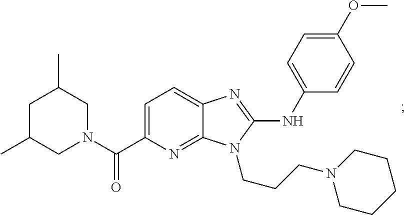 [SMILES: COc1ccc(Nc2nc3ccc(C(=O)N4CC(C)CC(C)C4)nc3n2CCCN2CCCCC2)cc1] |
| 3 | 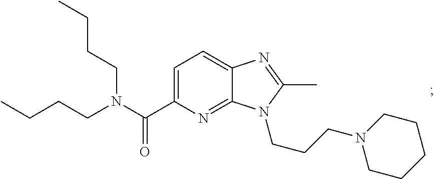 [SMILES: CCCCN(CCCC)C(=O)c1ccc2nc(C)n(CCCN3CCCCC3)c2n1] |
| 4 | 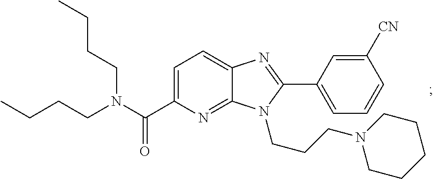 [SMILES: CCCCN(CCCC)C(=O)c1ccc2nc(-c3cccc(C#N)c3)n(CCCN3CCCCC3)c2n1] |
| 5 | 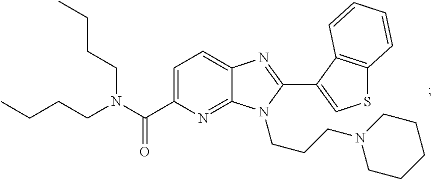 [SMILES: CCCCN(CCCC)C(=O)c1ccc2nc(-c3csc4ccccc34)n(CCCN3CCCCC3)c2n1] |
| 7 | 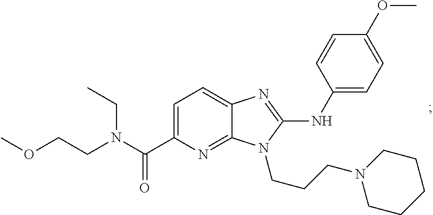 [SMILES: CCN(CCOC)C(=O)c1ccc2nc(Nc3ccc(OC)cc3)n(CCCN3CCCCC3)c2n1] |
| 8 | 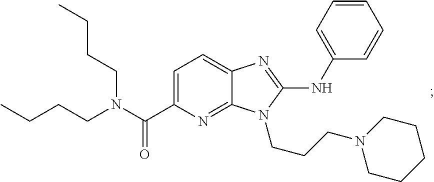 [SMILES: CCCCN(CCCC)C(=O)c1ccc2nc(Nc3ccccc3)n(CCCN3CCCCC3)c2n1] |
| 9 | 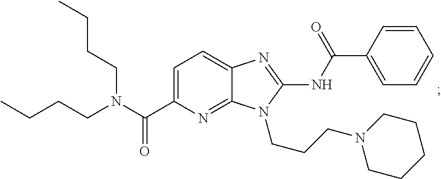 [SMILES: CCCCN(CCCC)C(=O)c1ccc2nc(NC(=O)c3ccccc3)n(CCCN3CCCCC3)c2n1] |
| 10 | 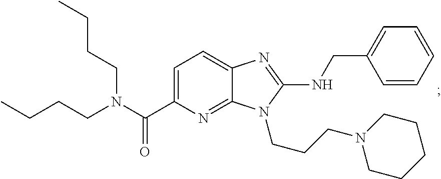 [SMILES: CCCCN(CCCC)C(=O)c1ccc2nc(NCc3ccccc3)n(CCCN3CCCCC3)c2n1] |
| 11 | 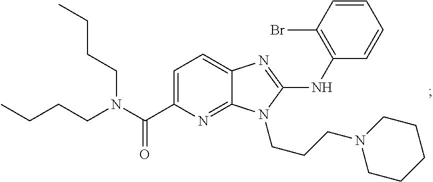 [SMILES: CCCCN(CCCC)C(=O)c1ccc2nc(Nc3ccccc3Br)n(CCCN3CCCCC3)c2n1] |
| 12 | 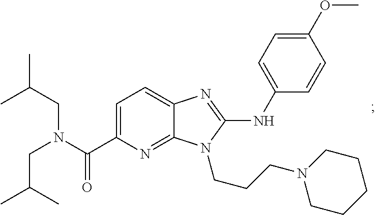 [SMILES: COc1ccc(Nc2nc3ccc(C(=O)N(CC(C)C)CC(C)C)nc3n2CCCN2CCCCC2)cc1] |
| 13 | 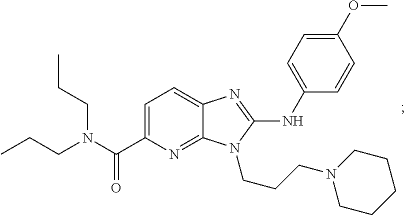 [SMILES: CCCN(CCC)C(=O)c1ccc2nc(Nc3ccc(OC)cc3)n(CCCN3CCCCC3)c2n1] |
| 14 | 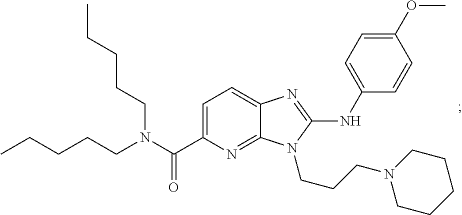 [SMILES: CCCCCN(CCCCC)C(=O)c1ccc2nc(Nc3ccc(OC)cc3)n(CCCN3CCCCC3)c2n1] |
| 15 | 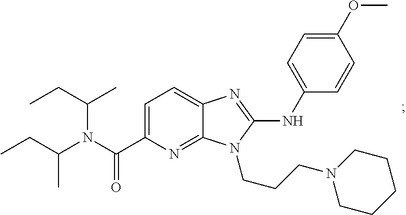 [SMILES: CCC(C)N(C(=O)c1ccc2nc(Nc3ccc(OC)cc3)n(CCCN3CCCCC3)c2n1)C(C)CC] |
| 16 | 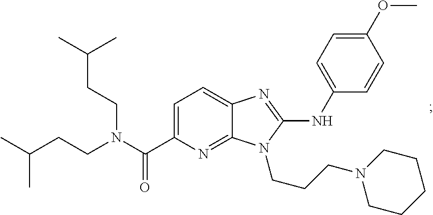 [SMILES: COc1ccc(Nc2nc3ccc(C(=O)N(CCC(C)C)CCC(C)C)nc3n2CCCN2CCCCC2)cc1] |
| 17 | 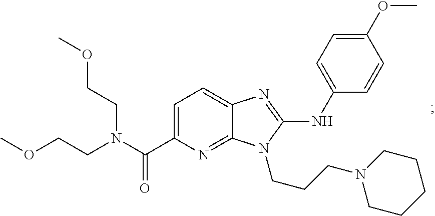 [SMILES: COCCN(CCOC)C(=O)c1ccc2nc(Nc3ccc(OC)cc3)n(CCCN3CCCCC3)c2n1] |
| 18 | 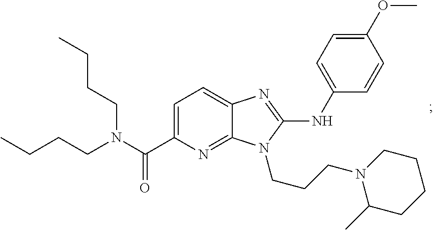 [SMILES: CCCCN(CCCC)C(=O)c1ccc2nc(Nc3ccc(OC)cc3)n(CCCN3CCCCC3C)c2n1] |
| 19 | 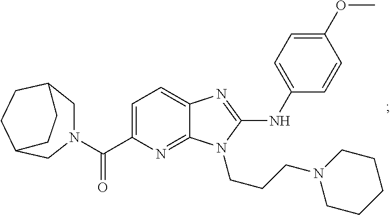 [SMILES: COc1ccc(Nc2nc3ccc(C(=O)N4CC5CCC(CC5)C4)nc3n2CCCN2CCCCC2)cc1] |
| 20 | 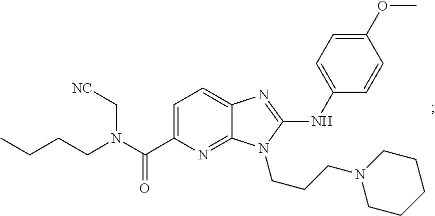 [SMILES: CCCCN(CC#N)C(=O)c1ccc2nc(Nc3ccc(OC)cc3)n(CCCN3CCCCC3)c2n1] |
| 21 | 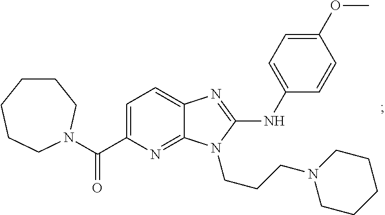 [SMILES: COc1ccc(Nc2nc3ccc(C(=O)N4CCCCCC4)nc3n2CCCN2CCCCC2)cc1] |
| 22 | 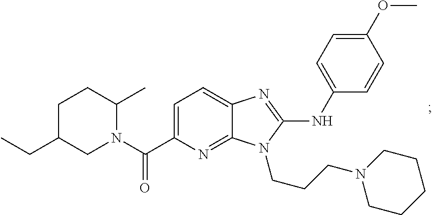 [SMILES: CCC1CCC(C)N(C(=O)c2ccc3nc(Nc4ccc(OC)cc4)n(CCCN4CCCCC4)c3n2)C1] |
| 23 | 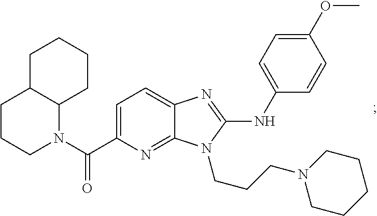 [SMILES: COc1ccc(Nc2nc3ccc(C(=O)N4CCCC5CCCCC54)nc3n2CCCN2CCCCC2)cc1] |
| 24 | 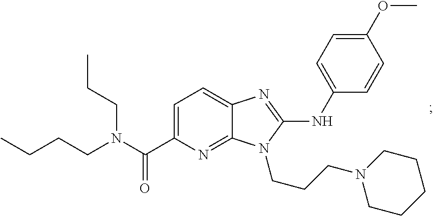 [SMILES: CCCCN(CCC)C(=O)c1ccc2nc(Nc3ccc(OC)cc3)n(CCCN3CCCCC3)c2n1] |
| 25 | 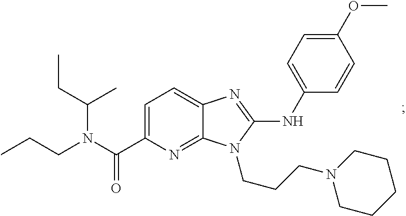 [SMILES: CCCN(C(=O)c1ccc2nc(Nc3ccc(OC)cc3)n(CCCN3CCCCC3)c2n1)C(C)CC] |
| 26 | 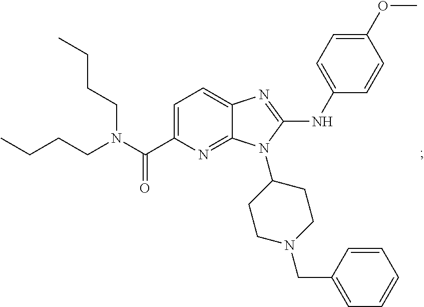 [SMILES: CCCCN(CCCC)C(=O)c1ccc2nc(Nc3ccc(OC)cc3)n(C3CCN(Cc4ccccc4)CC3)c2n1] |
| 27 | 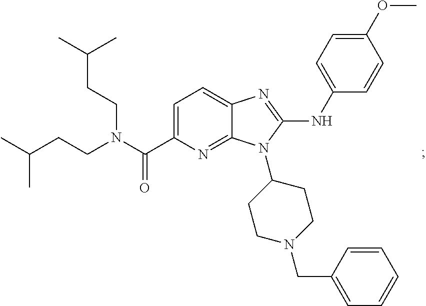 [SMILES: COc1ccc(Nc2nc3ccc(C(=O)N(CCC(C)C)CCC(C)C)nc3n2C2CCN(Cc3ccccc3)CC2)cc1] |
| 28 | 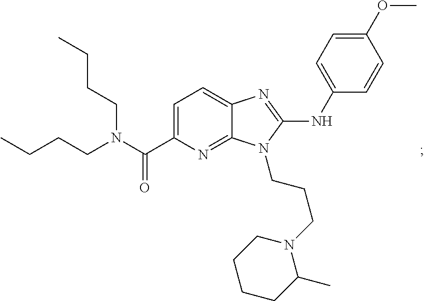 [SMILES: CCCCN(CCCC)C(=O)c1ccc2nc(Nc3ccc(OC)cc3)n(CCCN3CCCCC3C)c2n1] |
| 29 | 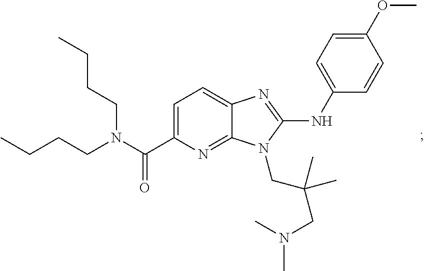 [SMILES: CCCCN(CCCC)C(=O)c1ccc2nc(Nc3ccc(OC)cc3)n(CC(C)(C)CN(C)C)c2n1] |
| 30 | 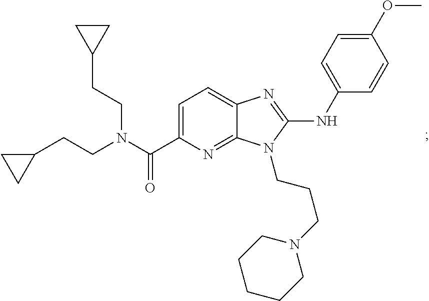 [SMILES: COc1ccc(Nc2nc3ccc(C(=O)N(CCC4CC4)CCC4CC4)nc3n2CCCN2CCCCC2)cc1] |
| 31 | 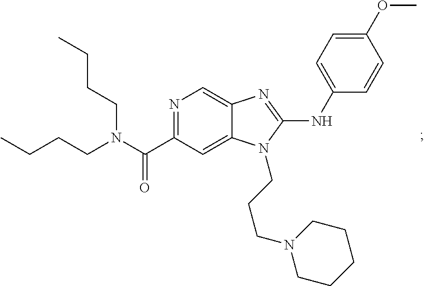 [SMILES: CCCCN(CCCC)C(=O)c1cc2c(cn1)nc(Nc1ccc(OC)cc1)n2CCCN1CCCCC1] |
| 32 | 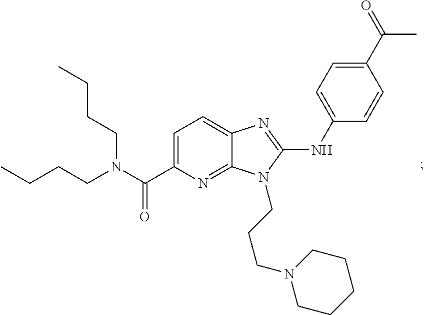 [SMILES: CCCCN(CCCC)C(=O)c1ccc2nc(Nc3ccc(C(C)=O)cc3)n(CCCN3CCCCC3)c2n1] |
| 33 | 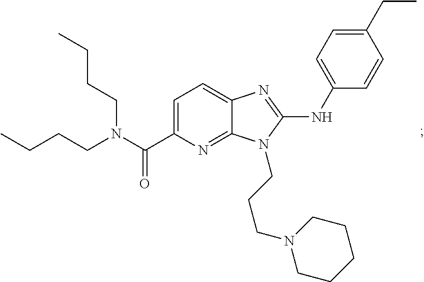 [SMILES: CCCCN(CCCC)C(=O)c1ccc2nc(Nc3ccc(CC)cc3)n(CCCN3CCCCC3)c2n1] |
| 34 | 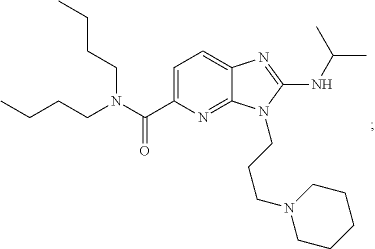 [SMILES: CCCCN(CCCC)C(=O)c1ccc2nc(NC(C)C)n(CCCN3CCCCC3)c2n1] |
| 35 | 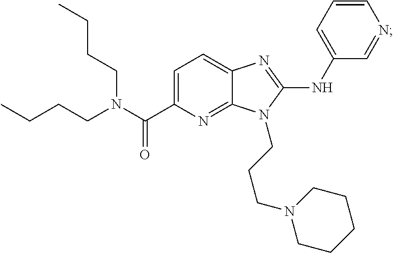 [SMILES: CCCCN(CCCC)C(=O)c1ccc2nc(Nc3cccnc3)n(CCCN3CCCCC3)c2n1] |
| 36 | 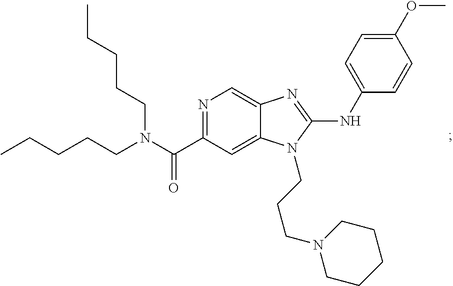 [SMILES: CCCCCN(CCCCC)C(=O)c1cc2c(cn1)nc(Nc1ccc(OC)cc1)n2CCCN1CCCCC1] |
| 37 | 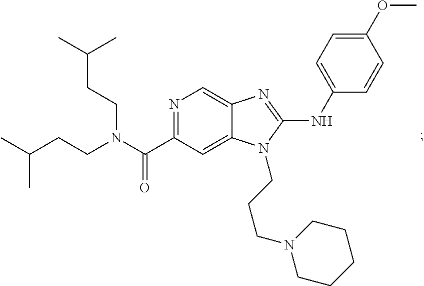 [SMILES: COc1ccc(Nc2nc3cnc(C(=O)N(CCC(C)C)CCC(C)C)cc3n2CCCN2CCCCC2)cc1] |
| 38 | 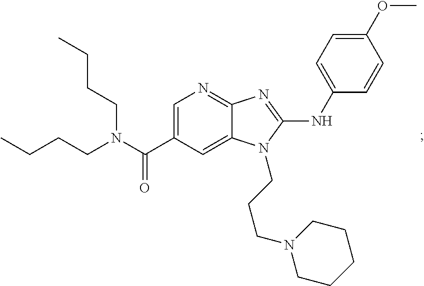 [SMILES: CCCCN(CCCC)C(=O)c1cnc2nc(Nc3ccc(OC)cc3)n(CCCN3CCCCC3)c2c1] |
| 39 | 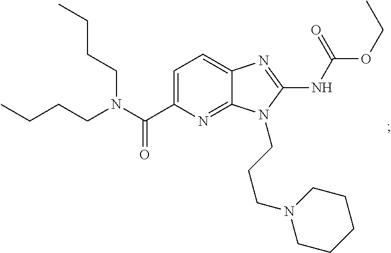 [SMILES: CCCCN(CCCC)C(=O)c1ccc2nc(NC(=O)OCC)n(CCCN3CCCCC3)c2n1] |
| 40 | 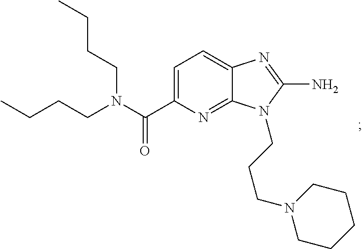 [SMILES: CCCCN(CCCC)C(=O)c1ccc2nc(N)n(CCCN3CCCCC3)c2n1] |
| 41 | 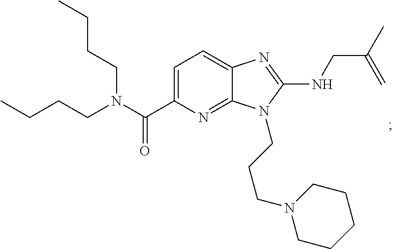 [SMILES: C=C(C)CNc1nc2ccc(C(=O)N(CCCC)CCCC)nc2n1CCCN1CCCCC1] |
| 42 | 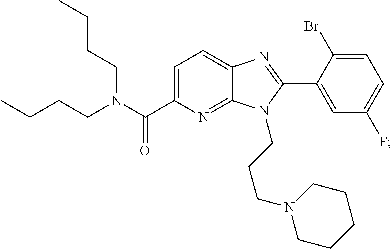 [SMILES: CCCCN(CCCC)C(=O)c1ccc2nc(-c3cc(F)ccc3Br)n(CCCN3CCCCC3)c2n1] |
| 43 | 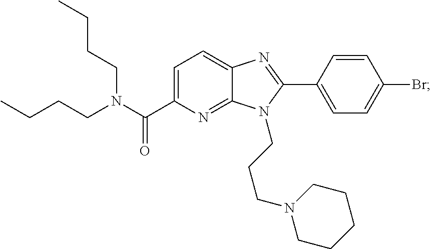 [SMILES: CCCCN(CCCC)C(=O)c1ccc2nc(-c3ccc(Br)cc3)n(CCCN3CCCCC3)c2n1] |
| 44 | 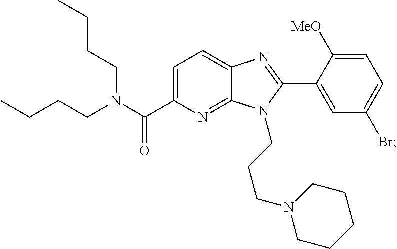 [SMILES: CCCCN(CCCC)C(=O)c1ccc2nc(-c3cc(Br)ccc3OC)n(CCCN3CCCCC3)c2n1] |
| 45 | 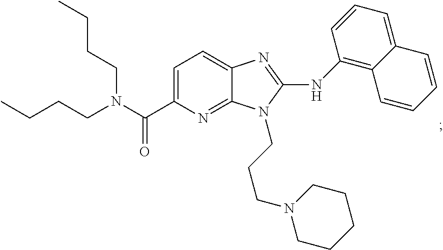 [SMILES: CCCCN(CCCC)C(=O)c1ccc2nc(Nc3cccc4ccccc34)n(CCCN3CCCCC3)c2n1] |
| 46 | 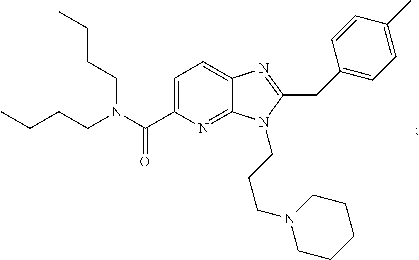 [SMILES: CCCCN(CCCC)C(=O)c1ccc2nc(Cc3ccc(C)cc3)n(CCCN3CCCCC3)c2n1] |
| 47 | 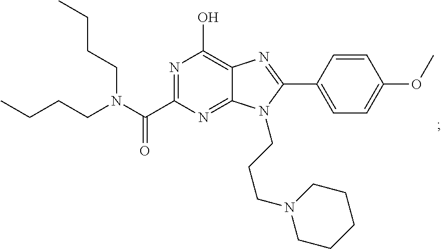 [SMILES: CCCCN(CCCC)C(=O)c1nc(O)c2nc(-c3ccc(OC)cc3)n(CCCN3CCCCC3)c2n1] |
| 48 | 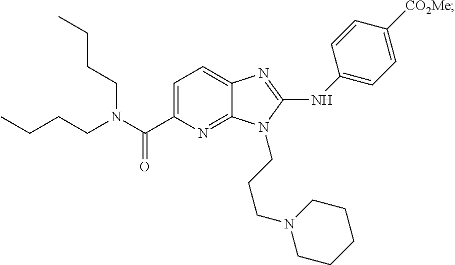 [SMILES: CCCCN(CCCC)C(=O)c1ccc2nc(Nc3ccc(C(=O)OC)cc3)n(CCCN3CCCCC3)c2n1] |
| 49 | 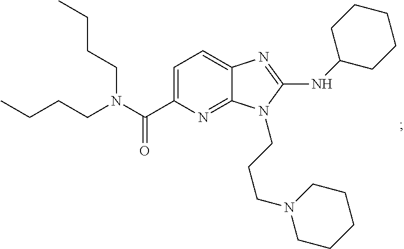 [SMILES: CCCCN(CCCC)C(=O)c1ccc2nc(NC3CCCCC3)n(CCCN3CCCCC3)c2n1] |
| 50 | 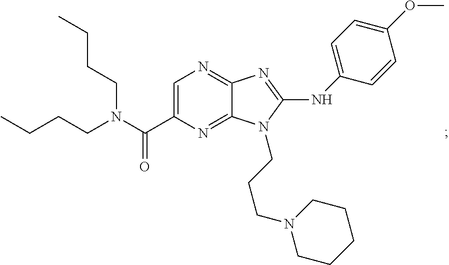 [SMILES: CCCCN(CCCC)C(=O)c1cnc2nc(Nc3ccc(OC)cc3)n(CCCN3CCCCC3)c2n1] |
| 51 | 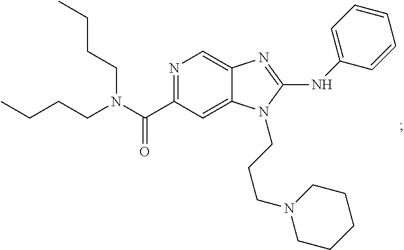 [SMILES: CCCCN(CCCC)C(=O)c1cc2c(cn1)nc(Nc1ccccc1)n2CCCN1CCCCC1] |
| 52 | 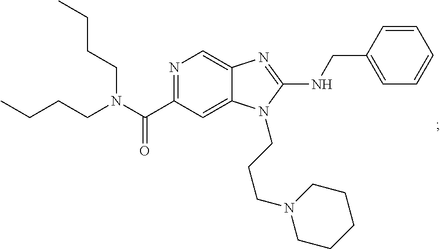 [SMILES: CCCCN(CCCC)C(=O)c1cc2c(cn1)nc(NCc1ccccc1)n2CCCN1CCCCC1] |
| 53 | 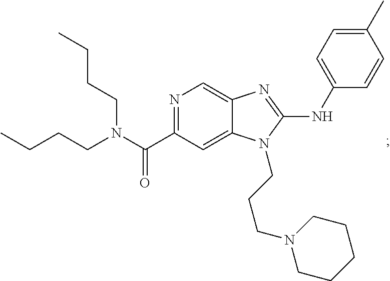 [SMILES: CCCCN(CCCC)C(=O)c1cc2c(cn1)nc(Nc1ccc(C)cc1)n2CCCN1CCCCC1] |
| 54 | 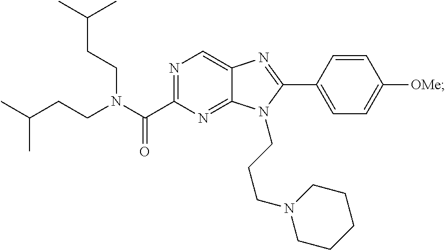 [SMILES: COc1ccc(-c2nc3cnc(C(=O)N(CCC(C)C)CCC(C)C)nc3n2CCCN2CCCCC2)cc1] |
| 55 | 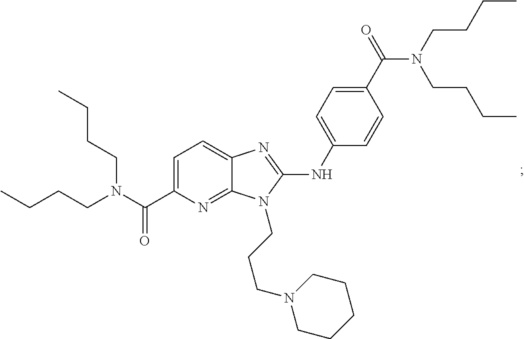 [SMILES: CCCCN(CCCC)C(=O)c1ccc(Nc2nc3ccc(C(=O)N(CCCC)CCCC)nc3n2CCCN2CCCCC2)cc1] |
| 56 | 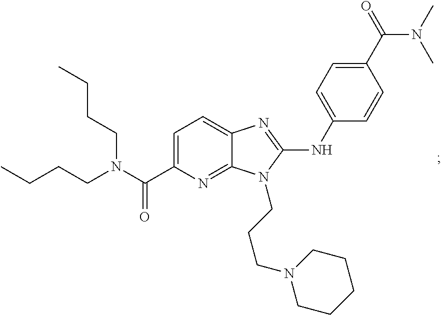 [SMILES: CCCCN(CCCC)C(=O)c1ccc2nc(Nc3ccc(C(=O)N(C)C)cc3)n(CCCN3CCCCC3)c2n1] |
| 57 | 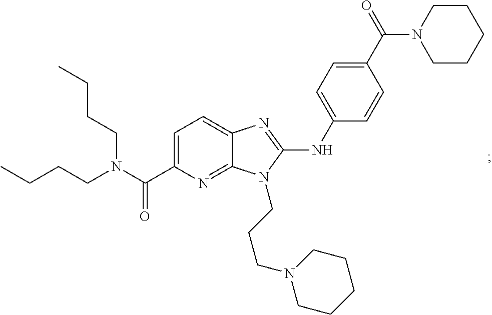 [SMILES: CCCCN(CCCC)C(=O)c1ccc2nc(Nc3ccc(C(=O)N4CCCCC4)cc3)n(CCCN3CCCCC3)c2n1] |
| 58 | 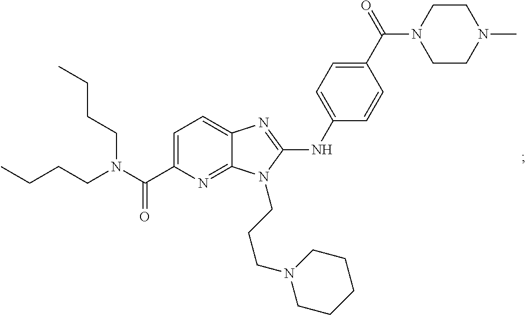 [SMILES: CCCCN(CCCC)C(=O)c1ccc2nc(Nc3ccc(C(=O)N4CCN(C)CC4)cc3)n(CCCN3CCCCC3)c2n1] |
| 59 | 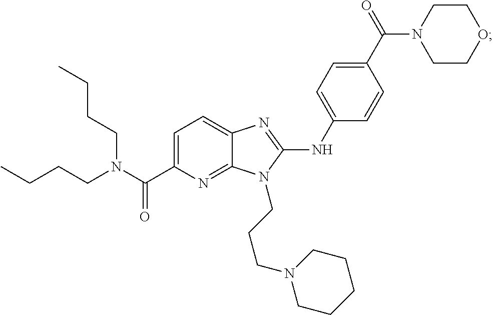 [SMILES: CCCCN(CCCC)C(=O)c1ccc2nc(Nc3ccc(C(=O)N4CCOCC4)cc3)n(CCCN3CCCCC3)c2n1] |
| 60 | 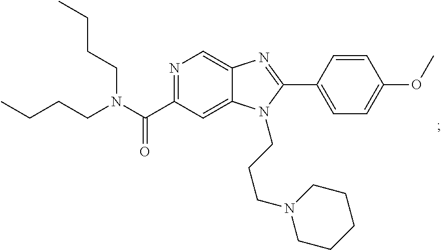 [SMILES: CCCCN(CCCC)C(=O)c1cc2c(cn1)nc(-c1ccc(OC)cc1)n2CCCN1CCCCC1] |
| 61 | 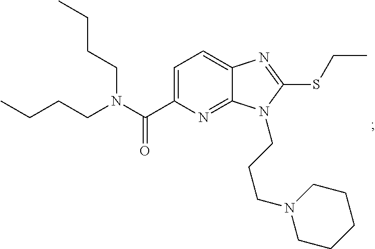 [SMILES: CCCCN(CCCC)C(=O)c1ccc2nc(SCC)n(CCCN3CCCCC3)c2n1] |
| 62 | 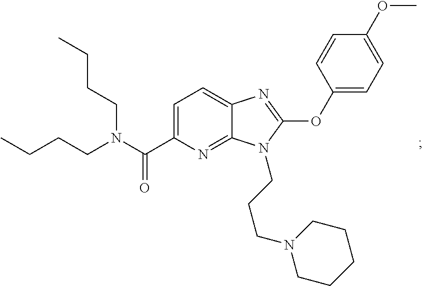 [SMILES: CCCCN(CCCC)C(=O)c1ccc2nc(Oc3ccc(OC)cc3)n(CCCN3CCCCC3)c2n1] |
| 63 | 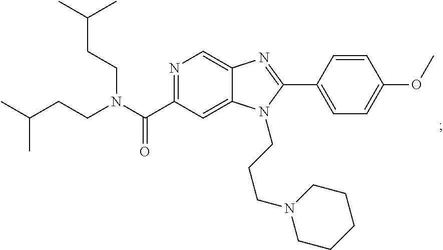 [SMILES: COc1ccc(-c2nc3cnc(C(=O)N(CCC(C)C)CCC(C)C)cc3n2CCCN2CCCCC2)cc1] |
| 64 | 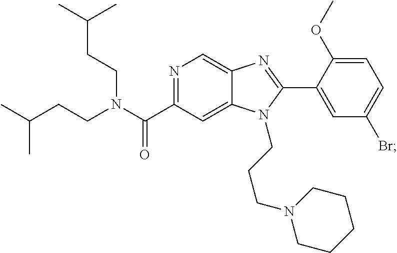 [SMILES: COc1ccc(Br)cc1-c1nc2cnc(C(=O)N(CCC(C)C)CCC(C)C)cc2n1CCCN1CCCCC1] |
| 65 | 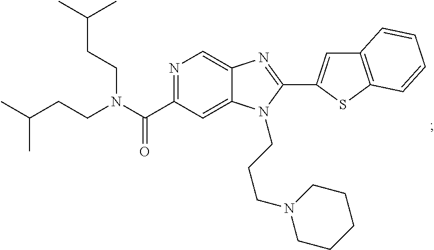 [SMILES: CC(C)CCN(CCC(C)C)C(=O)c1cc2c(cn1)nc(-c1cc3ccccc3s1)n2CCCN1CCCCC1] |
| 66 | 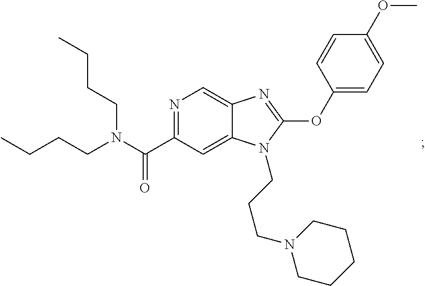 [SMILES: CCCCN(CCCC)C(=O)c1cc2c(cn1)nc(Oc1ccc(OC)cc1)n2CCCN1CCCCC1] |
| 67 | 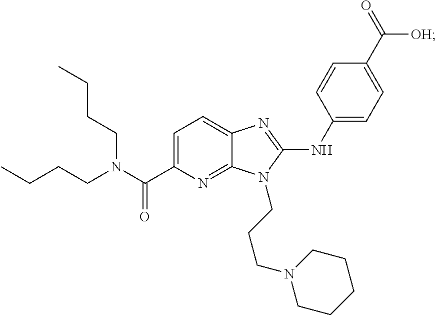 [SMILES: CCCCN(CCCC)C(=O)c1ccc2nc(Nc3ccc(C(=O)O)cc3)n(CCCN3CCCCC3)c2n1] |
| 68 | 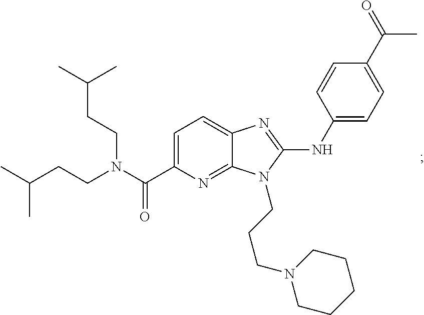 [SMILES: CC(=O)c1ccc(Nc2nc3ccc(C(=O)N(CCC(C)C)CCC(C)C)nc3n2CCCN2CCCCC2)cc1] |
| 69 | 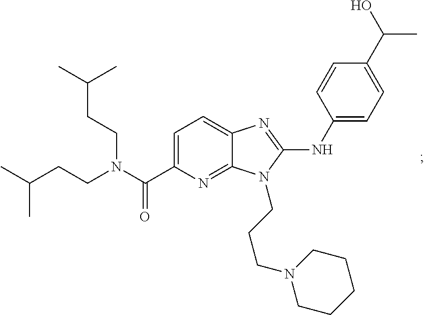 [SMILES: CC(C)CCN(CCC(C)C)C(=O)c1ccc2nc(Nc3ccc(C(C)O)cc3)n(CCCN3CCCCC3)c2n1] |
| 70 | 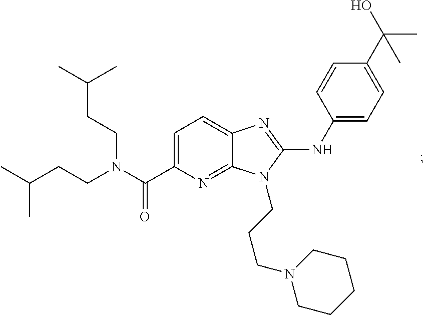 [SMILES: CC(C)CCN(CCC(C)C)C(=O)c1ccc2nc(Nc3ccc(C(C)(C)O)cc3)n(CCCN3CCCCC3)c2n1] |
| 71 | 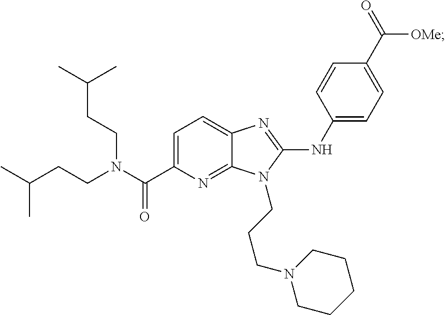 [SMILES: COC(=O)c1ccc(Nc2nc3ccc(C(=O)N(CCC(C)C)CCC(C)C)nc3n2CCCN2CCCCC2)cc1] |
| 72 | 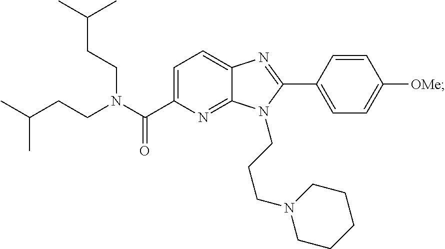 [SMILES: COc1ccc(-c2nc3ccc(C(=O)N(CCC(C)C)CCC(C)C)nc3n2CCCN2CCCCC2)cc1] |
| 73 | 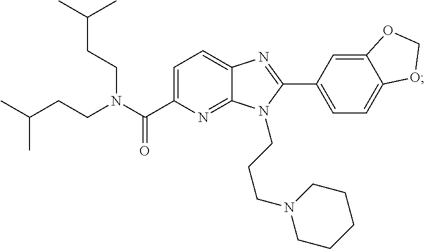 [SMILES: CC(C)CCN(CCC(C)C)C(=O)c1ccc2nc(-c3ccc4c(c3)OCO4)n(CCCN3CCCCC3)c2n1] |
| 74 | 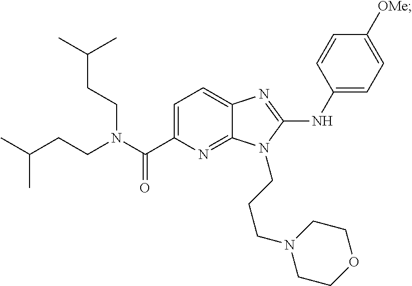 [SMILES: COc1ccc(Nc2nc3ccc(C(=O)N(CCC(C)C)CCC(C)C)nc3n2CCCN2CCOCC2)cc1] |
| 75 | 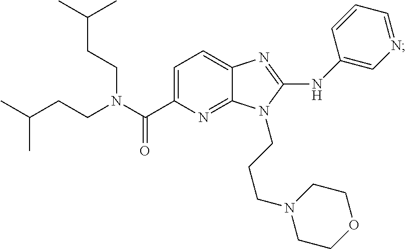 [SMILES: CC(C)CCN(CCC(C)C)C(=O)c1ccc2nc(Nc3cccnc3)n(CCCN3CCOCC3)c2n1] |
| 76 | 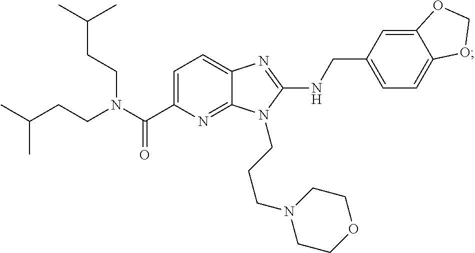 [SMILES: CC(C)CCN(CCC(C)C)C(=O)c1ccc2nc(NCc3ccc4c(c3)OCO4)n(CCCN3CCOCC3)c2n1] |
| 77 | 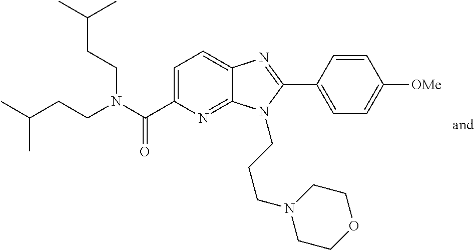 [SMILES: COc1ccc(-c2nc3ccc(C(=O)N(CCC(C)C)CCC(C)C)nc3n2CCCN2CCOCC2)cc1] |
| 78 | 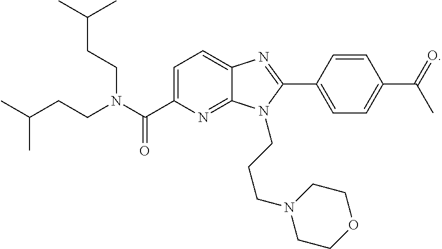 [SMILES: CC(=O)c1ccc(-c2nc3ccc(C(=O)N(CCC(C)C)CCC(C)C)nc3n2CCCN2CCOCC2)cc1] |

16. The method of claim 1, wherein the subject is an animal or a human.

17. The method of claim 16, wherein said subject has a disorder selected from the group consisting of obesity, cachexia, eating disorders, diabetes, metabolic disease, erectile dysfunction and sexual dysfunction.

18. The method of claim 17, in which the disorder is obesity.

19. A method of treating obesity in a subject, the method comprising administering to said subject in need thereof a therapeutically effective amount of the compound as defined in claim 1.
